(19) United States
(12) Patent Application Publication
(10) Pub. No.: US 2023/0045277 A1
(43) Pub. Date: Feb. 9, 2023

(54) COMPOUNDS, POLYMERS, DEVICES, AND USES THEREOF

(71) Applicant: SIGILON THERAPEUTICS, INC., Cambridge, MA (US)

(72) Inventors: Elyse Bourque, Bellingham, WA (US); Richard Heidebrecht, Somerville, MA (US); Christopher P. Hencken, Boston, MA (US)

(21) Appl. No.: 17/784,972

(22) PCT Filed: Dec. 11, 2020
(86) PCT No.: PCT/US2020/064661

§ 371 Date: Jun. 13, 2022

Related U.S. Application Data
(60) Provisional application No. 62/947,951, filed on Dec. 13, 2019.

(51) Int. Cl. A61K 47/54; A61K 9/00; C07D 417/06; C07D 403/06

(52) U.S. Cl. CPC A61K 47/545; A61K 9/0024; C07D 417/06; C07D 403/06

(57) ABSTRACT

Described herein are compounds of Formula (I), polymers and implantable elements comprising compounds of Formula (II), and compositions and methods of use thereof. In particular, the compounds, modified polymers, implantable elements and related compositions may be used in methods for the prevention and treatment of a disease, disorder or condition in a subject.

### BACKGROUND

The function of implanted devices depends in large part on the biological immune response pathway of the recipient (Anderson et al., Semin. Immunol. 20:86-100 (2008); Langer, Adv. Mater. 21:3235-3236 (2009)). Modulation of the immune response may impart a beneficial effect on the fidelity and function of these devices. As such, there is a need in the art for new compounds, compositions, and devices that achieve this goal.

### SUMMARY

Described herein are compounds of Formula (I), compounds of Formula (II), polymers modified with a compound(s) of Formula (II) and implantable elements comprising a compound of Formula (II), as well as compositions and methods of use thereof. In particular, the compounds, modified polymers, implantable elements and related compositions may be used in methods for the prevention and treatment of a disease, disorder or condition in a subject.

In one aspect, the disclosure features a compound of Formula (I):

A-L¹-M-L²-P-L³-Z (I)

or a pharmaceutically acceptable salt thereof, wherein the variables A, L¹, M, L², P, L³, Z, and subvariables thereof are defined herein. In some embodiments, the compound of Formula (I) or a pharmaceutically acceptable salt thereof (e.g., a compound of Formulas (I-a), (I-b), (I-c), (I-d), (I-e), (I-f), (I-g), (I-h), (I-i), (I-j), (I-k), or (I-l)) is one of the compounds shown in Table 1 herein.

In another aspect, the disclosure features a polymer modified with a compound of Formula (II):

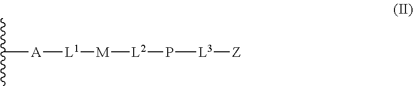

or a pharmaceutically acceptable salt thereof, wherein the variables A, L¹, M, L², P, L³, Z, and subvariables thereof are defined herein. In some embodiments, the polymer is a polysaccharide, e.g., alginate, hyaluronate, or chitosan. In some embodiment, the polymer is alginate. In some embodiments, the compound of Formula (II) or a pharmaceutically acceptable salt thereof (e.g., a compound of Formulas (II-a), (II-b), (II-c), (II-d), (II-e), (II-f), (II-g), (II-h), (II-i), (II-j), (II-k), or (II-l)) is one of the compounds shown in Table 2 herein.

In another aspect, the disclosure features an implantable element (e.g., a device or material) comprising a compound of Formula (II), or a pharmaceutically acceptable salt thereof, as described herein. In some embodiments, the compound is associated with (e.g., covalently bound to) a surface of the implantable element. In other embodiments, the implantable element comprises a polymer modified with a compound of Formula (II). In some embodiments, the compound of Formula (II) or a pharmaceutically acceptable salt thereof (e.g., a compound of Formulas (II-a), (II-b), (II-c), (II-d), (II-e), (II-f), (II-g), (II-h), (II-i), (II-j), (II-k), or (II-l)) is one of the compounds shown in Table 2 herein.

In some embodiments, the implantable element comprises a cell. Exemplary cell types include epithelial cells, endothelial cells, fibroblasts, keratinocytes, and mesenchymal stem cells (MSCs). In some embodiments, the implantable element comprises an epithelial cell, e.g., a retinal pigment epithelial cell (RPE cell). In some embodiments, the implantable element comprises an engineered cell (e.g., an engineered epithelial cell, e.g., an engineered RPE cell).

In some embodiments, the cell (e.g., an engineered cell) produces a substance, e.g., a therapeutic agent. Exemplary therapeutic agents include a nucleic acid (e.g., an RNA or DNA), protein (e.g., a hormone, enzyme, antibody, antibody fragment, antigen, or epitope), small molecule, lipid, drug, vaccine, or any derivative thereof. For example, an implantable element may comprise an engineered cell capable of producing a protein (e.g., a blood clotting factor (e.g., a Factor VIII protein) or a hormone (e.g, insulin)).

In another aspect, the disclosure features a method of providing a substance (e.g., a therapeutic agent) to a subject, comprising administering to the subject an implantable element comprising (i) a compound of Formula (II), as described herein, and (ii) a cell capable of producing the substance (e.g., therapeutic agent). In some embodiments, the substance is a therapeutic agent, e.g., a protein (e.g., a blood clotting factor (e.g., a Factor VIII protein) or a hormone (e.g., insulin)).

In another aspect, the disclosure features a method of treating a disease, disorder, or condition in a subject with a therapeutic agent that is capable of treating the disease, disorder or condition, the method comprising administering to the subject an implantable element comprising (i) a compound of Formula (II), as described herein, and (ii) a cell capable of producing the therapeutic agent. In some embodiments, the disorder is a blood clotting disorder (e.g., Hemophilia A), a lysosomal storage disorder (e.g., Fabry Disease, MPS I), an endocrine disorder, diabetes, or a neurodegenerative disease.

In some embodiments, the method or providing a substance or method of treating comprises reducing the foreign body response to the administered implantable element (e.g., minimizing the formation of pericapsular fibrotic overgrowth (PFO) on the implantable element).

In any and all aspects of the disclosure, in some embodiments the compound of Formula (I), a polymer modified with a compound of Formula (II), or an implantable element (e.g., device or material) comprising a compound of Formula (II) is not a compound, polymer, or implantable element described in any one of WO2012/112982, WO2012/167223, WO2014/153126, WO2016/187225, WO2016/019391, WO2017/075630, WO 2017/075631, WO 2018/067615, WO 2019/169333, and US 2016-0030359. In some embodiments, the compound of Formula (II) is attached to a polymer or implantable element (e.g., device or material) through an attachment group other than an attachment group described in any one of WO2012/112982, WO2012/167223, WO2014/153126, WO2016/187225, WO2016/019391, WO2017/075630, WO 2017/075631, WO 2018/067615, WO 2019/169333, and US 2016-0030359.

The details of one or more embodiments of the invention are set forth herein. Other features, objects, and advantages of the invention will be apparent from the Detailed Description, the Figures, the Examples, and the Claims.

### DETAILED DESCRIPTION

The disclosure provides a compound, e.g., a compound of Formula (I) or Formula (II), polymers modified with a compound of Formula (II), and implantable elements (e.g., devices and materials) comprising a compound of Formula (II), as well as related compositions and methods of use thereof. In particular, the compounds, polymers and implantable elements described herein may be used in methods for the prevention and treatment of a disease, disorder or condition in a subject. In some embodiments, the compounds of Formula (I), and polymers and implantable elements comprising a compound of Formula (II), as well as pharmaceutically acceptable salts, solvates, hydrates, tautomers, stereoisomers, isotopically labeled derivatives thereof, are capable of mitigating the immune response in a subject.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About”, when used herein to modify a numerically defined parameter (e.g., a physical description of a polymer or implantable element as described herein, such as diameter, sphericity, number of cells in a particle (e.g., hydrogel capsule), the number of particles in a preparation), means that the parameter may vary by as much as 15% above or below the stated numerical value for that parameter. For example, an implantable element defined as having a mean diameter of about 1.5 millimeters (mm) and encapsulating about 5 million (M) cells may have a mean diameter of 1.275 to 1.725 mm and may encapsulate about 4.25 M to 5.75 M cells. In some embodiments, the term “about” means that the parameter may vary by as much as 10% or 5% above or below the stated numerical value for that parameter.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., fluorescence microscope to acquire fluorescence microscopy data.

“Administer”, “administering”, or “administration”, as used herein, refer to implanting, absorbing, ingesting, injecting, disposing or otherwise introducing into a subject an entity described herein (e.g., an implantable element, e.g., a particle comprising a first compartment, a second compartment, and a compound of Formula (I) or Formula (II) (including particles encapsulating cells, e.g., engineered RPE cells), or a composition comprising said particles), or providing the entity to a subject for administration.

“Afibrotic”, as used herein, means a compound or material that mitigates at least one aspect of the foreign body response (FBR) to an implant comprising the compound or material, e.g., minimizes the formation of pericapsular fibrotic overgrowth (PFO) on the implant. For example, the FBR in a biological tissue or tissue fluid that is induced by implant into that tissue or tissue fluid of a polymer or device (e.g., hydrogel capsule) comprising an afibrotic compound (e.g., a hydrogel capsule comprising a polymer covalently modified with a compound listed in Table 1 or Table 2) occurs in a lower amount, or at a later time, than the FBR induced by implantation of an afibrotic-null reference polymer or device, i.e., lacks any afibrotic compound, but otherwise has substantially the same composition (e.g., hydrogel capsule formed from the same non-modified polymer, and having substantially the same shape and size). In an embodiment, the degree of the FBR is assessed by the immunological response in the tissue or tissue fluid containing the implanted device (e.g., hydrogel capsule), which may include, for example, protein adsorption, macrophages, multinucleated foreign body giant cells, eosinophils, neutrophils, T cells, B cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), granulocytes (Siglec-F, Ly-6G), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse. In an embodiment, the FBR is assessed by measuring the levels in the tissue or tissue fluid containing the implant of one or more biomarkers of immune response, e.g., cathepsin, TNF-α, IL-13, IL-6, G-CSF, GM-CSF, IL-1, IL-4, IL-5, CCL2, CCL4, TIMP-1. In some embodiments, the FBR is assessed by examining the amount of PFO on the implant (e.g., hydrogel capsule) at one or more times following the administration to suitable test subjects (e.g., immunocompetent mice); this assessment can be done using assays known in the art, e.g., any of the assays described in this definition. In some embodiments, an aspect of the FBR (e.g., PFO) induced by a modified polymer or device of the invention (e.g., a hydrogel capsule comprising an afibrotic compound described herein disposed on its outer surface), is at least about 80%, about 85%, about 90%, about 95%, about 99%, or about 100% lower than, or occurs at least about 10%, about 20%, about 40% or about 50% later than, the same FBR aspect induced by an afibrotic-null reference polymer or device. In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

“Cell,” as used herein, refers to an engineered cell or a cell that is not engineered.

“Effective amount” as used herein refers to an amount of a compound, modified polymer, or implantable element described herein, e.g, further comprising a cell, e.g., an engineered cell, or an agent, e.g., a therapeutic agent, produced by a cell, e.g., an engineered cell, sufficient to mitigate or elicit a biological response, e.g., minimize an immune response, or to treat a disease, disorder, or condition. As will be appreciated by those of ordinary skill in this art, the effective amount may vary depending on such factors as the desired biological endpoint, the pharmacokinetics of the therapeutic agent, composition or implantable element, the condition being treated, the mode of administration, and the age and health of the subject. An effective amount encompasses therapeutic and prophylactic treatment. For example, to reduce the foreign body response (e.g., PFO) induced by an implantable element, a compound described herein may be disposed on the surface of the implantable element in an amount effective to reduce the PFO or stop the growth or spread of fibrotic tissue on or near the implantable element.

An “endogenous nucleic acid” as used herein, is a nucleic acid that occurs naturally in a subject cell.

An “endogenous polypeptide,” as used herein, is a polypeptide that occurs naturally in a subject cell.

“Engineered cell,” as used herein, is a cell having a non-naturally occurring alteration, and typically comprises a nucleic acid sequence (e.g., DNA or RNA) or a polypeptide not present (or present at a different level than) in an otherwise similar cell under similar conditions that is not engineered (an exogenous nucleic acid sequence). In an embodiment, an engineered cell comprises an exogenous nucleic acid (e.g., a vector or an altered chromosomal sequence). In an embodiment, an engineered cell comprises an exogenous polypeptide. In an embodiment, an engineered cell comprises an exogenous nucleic acid sequence, e.g., a sequence, e.g., DNA or RNA, not present in a similar cell that is not engineered. In an embodiment, the exogenous nucleic acid sequence is chromosomal, e.g., the exogenous nucleic acid sequence is an exogenous sequence disposed in endogenous chromosomal sequence. In an embodiment, the exogenous nucleic acid sequence is chromosomal or extra chromosomal, e.g., a non-integrated vector. In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a chromosomal or extra-chromosomal nucleic acid sequence, which comprises a sequence that encodes a polypeptide, or which is expressed as a polypeptide. In an embodiment, the exogenous nucleic acid sequence comprises a first chromosomal or extra-chromosomal exogenous nucleic acid sequence that modulates the conformation or expression of a second nucleic acid sequence, wherein the second amino acid sequence can be exogenous or endogenous. For example, an engineered cell can comprise an exogenous nucleic acid that controls the expression of an endogenous sequence. In an embodiment, an engineered cell comprises a polypeptide present at a level or distribution which differs from the level found in a similar cell that has not been engineered. In an embodiment, an engineered cell comprises a cell engineered to provide an RNA or a polypeptide. For example, an engineered cell may comprise an exogenous nucleic acid sequence comprising a chromosomal or extra-chromosomal exogenous nucleic acid sequence that comprises a sequence which is expressed as RNA, e.g., mRNA or a regulatory RNA. In an embodiment, an engineered cell comprises an exogenous nucleic acid sequence that comprises a chromosomal or extra-chromosomal nucleic acid sequence comprising a sequence that encodes a polypeptide, or which is expressed as a polypeptide. In an embodiment, an engineered cell comprises an exogenous nucleic acid sequence that modulates the conformation or expression of an endogenous sequence. In an embodiment, an engineered cell (e.g., RPE cell) is cultured from a population of stably transfected cells, or from a monoclonal cell line.

An “exogenous nucleic acid,” as used herein, is a nucleic acid that does not occur naturally in a subject cell, e.g., an engineered cell.

An “exogenous polypeptide,” as used herein, is a polypeptide that does not occur naturally in a subject cell, e.g., an engineered cell.

An “implantable element” as used herein, comprises a cell, e.g., a plurality of cells, e.g., a cluster of cells, wherein the cell or cells are entirely or partially disposed within an enclosing component (which enclosing component is other than a cell), e.g., the enclosing component comprises a non-cellular component. The term “implantable element” comprises a device or material described herein. In an embodiment, the implantable element inhibits an immune attack, or the effect of the immune attack, on the enclosed cell or cells. In an embodiment, the implantable element comprises a semipermeable membrane or a semipermeable polymer matrix or coating. Typically, the implantable element allows passage of small molecules, e.g., nutrients and waste products. Typically, the implantable element allows passage of a product (e.g., a therapeutic polypeptide) released by a cell disposed within the enclosing component. In an embodiment, placement within an implantable element minimizes an effect of a host response (e.g., an immune response, e.g., a fibrotic response) directed at the implantable element, e.g., against a cell within an implantable element, e.g., as compared with a similar cell that is not disposed in an implantable element. The implantable element described herein comprises a compound of Formula (II) or a pharmaceutically acceptable salt thereof, that minimizes an effect of an immune response, e.g., a fibrotic response, of the subject directed at the implantable element, e.g., against the enclosing component or a cell within the implantable element, e.g., as compared with a similar or otherwise identical implantable element lacking the compound. In some embodiments, the implantable element (e.g., a device or material) is associated (e.g., directly associated) with a compound described herein, e.g., a compound of Formula (II). In some embodiments, the compound of Formula (II) is directly bound to the implantable element (e.g., a device or material). In some embodiments, the implantable element (e.g., a device or material) comprises a polymer modified with a compound of Formula (II).

“Pericapsular fibrotic overgrowth” or “PFO”, as used herein, refers to a fibrotic cell layer that forms on part or all of an implantable element (e.g., a hydrogel capsule) as a result of the foreign body response to the implantable element.

“Polypeptide”, as used herein, refers to a polymer comprising amino acid residues linked through peptide bonds and having at least two, and in embodiments, at least 10, 100, or 200 amino acid residues.

“Prevention,” “prevent,” and “preventing” as used herein refers to a treatment that comprises administering or applying a therapy, e.g., administering a composition of implantable elements encapsulating cells (e.g., as described herein), prior to the onset of a disease, disorder, or condition to preclude the physical manifestation of said disease, disorder, or condition. In some embodiments, “prevention,” “prevent,” and “preventing” require that signs or symptoms of the disease, disorder, or condition have not yet developed or have not yet been observed.

A “replacement therapy” or “replacement protein” is a therapeutic protein or functional fragment thereof that replaces or augments a protein that is diminished, present in insufficient quantity, altered (e.g., mutated) or lacking in a subject having a disease or condition related to the diminished, altered or lacking protein. Examples are certain blood clotting factors in certain blood clotting disorders or certain lysosomal enzymes in certain lysosomal storage diseases. In an embodiment, a replacement therapy or replacement protein provides the function of an endogenous protein. In an embodiment, a replacement therapy or replacement protein has the same amino acid sequence of a naturally occurring variant, e.g., a wild type allele or an allele not associated with a disorder, of the replaced protein. In an embodiment, or replacement therapy or a replacement protein differs in amino acid sequence from a naturally occurring variant, e.g., a wild type allele or an allele not associated with a disorder, e.g., the allele carried by a subject, at no more than about 1, 2, 3, 4, 5, 10, 15 or 20% of the amino acid residues.

“Subject” as used herein refers to a human or non-human animal. In an embodiment, the subject is a human (i.e., a male or female, e.g., of any age group, a pediatric subject (e.g., infant, child, adolescent) or adult subject (e.g., young adult, middle-aged adult, or senior adult)). In an embodiment, the subject is a non-human animal, for example, a mammal (e.g., a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., considering a history of symptoms and/or in light of genetic or other susceptibility factors). Treatment may also be continued after symptoms have resolved, for example, to delay or prevent recurrence. In some embodiments, treatment comprises prevention and in other embodiments it does not.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen, phosporous, silicon, or sulfur atoms may optionally be oxidized, and the nitrogen heteroatom may optionally be quaternized. The heteroatom(s) 0, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆ alkylene, C₂-C₆ alkenylene, C₂-C₆ alkynylene, or C₁-C₆ heteroalkylene. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₅), cubanyl (C₅), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR^{C}R^{D}, wherein R^{C} and R^{D} are each independently hydrogen, C₁-C₁₂ alkyl, C₃-C₁₀ cycloalkyl, C₃-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present invention contemplates any and all such combinations to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) or Formula (II) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) or Formula (II) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and 180; and the like.

The term “pharmaceutically acceptable salt” is meant to include salts of the active compounds that are prepared with relatively nontoxic acids or bases, depending on the particular substituents found on the compounds described herein. When compounds used in the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

In addition to salt forms, the disclosure may employ compounds of Formula (I) or Formula (II) in a prodrug form. Prodrugs are those compounds that readily undergo chemical changes under physiological conditions to provide the compounds useful in the present invention. Additionally, prodrugs can be converted to useful compounds of Formula (I) or Formula (II) by chemical or biochemical methods in an ex vivo environment.

Certain compounds of Formula (I) or Formula (II) described herein can exist in unsolvated forms as well as solvated forms, including hydrated forms. In general, the solvated forms are equivalent to unsolvated forms and are encompassed within the scope of the present invention. Certain compounds of Formula (I) or Formula (II) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, dimethylsulfoxide (DMSO), tetrahydrofuran (THF), diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of 7 electrons and an atom (usually H). For example, enols and ketones are tautomers because they are rapidly interconverted by treatment with either acid or base. Tautomeric forms may be relevant to the attainment of the optimal chemical reactivity and biological effect of a compound of interest.

The symbol “ [symbol] ” as used herein refers to a connection to an entity, e.g., a polymer (e.g., hydrogel-forming polymer such as alginate) or an implantable element (e.g., a device or material). The connection represented by “ [symbol] ” may refer to direct attachment to the entity, e.g., a polymer or an implantable element, or may refer to linkage to the entity through an attachment group. An “attachment group,” as described herein, refers to a moiety for linkage of a compound of Formula (II) to an entity (e.g., a polymer or an implantable element as described herein), and may comprise any attachment chemistry known in the art. A listing of exemplary attachment groups is outlined in Bioconjugate Techniques (3^{rd} ed, Greg T. Hermanson, Waltham, Mass.: Elsevier, Inc, 2013). In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)—, —OS(O)ₓ, —N(R^{C})S(O)ₓ, —S(O)ₓN(R^{C})—, —P(R^{F})_{y}—, —Si(OR^{A})₂—, —Si(R^{G})(OR^{A})—, —B(OR^{A}—, or a metal, wherein each of R^{A}, R^{C}, R^{D}, R^{F}, R^{G}, x and y is independently as described herein. In some embodiments, an attachment group comprises an amine, ketone, ester, amide, alkyl, alkenyl, alkynyl, or thiol. In some embodiments, an attachment group is a cross-linker. In some embodiments, the attachment group is —C(O)(C₁-C₆-alkylene)-, wherein alkylene is substituted with R¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

Compounds of Formula (I)

The present invention features a compound of Formula (I):

A-L¹-M-L²-P-L³-Z (I)

or a pharmaceutically acceptable salt thereof, wherein:

A is hydrogen, alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{c})C(O)(C₁-C₆-alkenylene)-, —N(R^{C})N(R^{D})—, —NCN—, —C(═N(R^{C})(R^{D}))O—, —S—, —S(O)ₓ-, —OS(O)ₓ, —N(R^{C})S(O)ₓ, —S(O)ₓN(R^{C})—, —P(R^{F})_{y}, —Si(OR^{A})₂—, —Si(R^{G})(OR^{A}), —B(OR^{A}), or a metal, wherein each alkyl, alkenyl, alkynyl, alkylene, alkenylene, heteroalkyl, cycloalkyl, heterocyclyl, aryl, and heteroaryl is linked to an attachment group (e.g., an attachment group defined herein) and is optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is absent, cycloalkyl, heterocycyl, or heteroaryl, each of which is optionally substituted by one or more R⁴;

Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently deuterium, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷; each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, the present invention features a compound of Formula (I-a):

A-L¹-M-L²-P-L³-Z (I-a)

or a pharmaceutically acceptable salt thereof, wherein:

A is hydrogen, alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ, —S(O)ₓN(R^{C})—, —P(R^{F})_{y}, —Si(OR^{A})₂—, —Si(R^{G})(OR^{A})—, —B(OR^{A})—, or a metal, wherein each alkyl, alkenyl, alkynyl, alkylene, alkenylene, heteroalkyl, cycloalkyl, heterocyclyl, aryl, and heteroaryl is linked to an attachment group (e.g., an attachment group defined herein) and is optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is absent, cycloalkyl, heterocycyl, or heteroaryl, each of which is optionally substituted by one or more R⁴;

Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;

each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, the compound of Formula (I) or (I-a) is a compound of Formula (I-b).

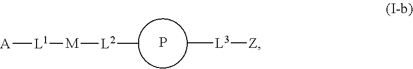

or a pharmaceutically acceptable salt thereof, wherein:

A is hydrogen, alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ—, —OS(O)ₓ, —N(R^{C})S(O)ₓ, —S(O)ₓN(R^{C})—, —P(R^{F})_{y}, —Si(OR^{A})₂—, —Si(R^{G})(OR^{A})—, —B(OR^{A}), or a metal, wherein each alkyl, alkenyl, alkynyl, alkylene, alkenylene, heteroalkyl, cycloalkyl, heterocyclyl, aryl, and heteroaryl is linked to an attachment group (e.g., an attachment group defined herein) and is optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is heteroaryl optionally substituted by one or more R⁴;

Z is alkyl, alkenyl, alkynyl, heteroalkyl, cycloalkyl, heterocyclyl, aryl, or heteroaryl, each of which is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), —SR^{E1}, —S(O)ₓR^{E1}, —OS(O)ₓR^{E1}, —N(R^{C1})S(O)ₓR^{E1}, —S(O)ₓN(R^{C1})(R^{D1}), —P(R^{F1})_{y} cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;

each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, for Formulas (I), (I-a), and (I-b), A is hydrogen, alkyl, alkenyl, —OR^{A}, —C(O)OR^{A}, —C(O)R^{B}, —N(R^{C})(R^{D}), —N(R^{C})C(O)R^{B}, —N(R^{C})C(O)(C₁-C₆-alkyl), or —N(R^{C})C(O)(C₁-C₆-alkenyl). In some embodiments, A is hydrogen, alkyl, alkenyl, N(R^{C})(R^{D}), —N(R^{C})C(O)(C₁-C₆-alkyl), or —N(R^{C})C(O)(C₁-C₆-alkenyl). In some embodiments, A is hydrogen. In some embodiments, A is —N(R^{C})(R^{D}), —N(R^{C})C(O)R^{B}, —N(R^{C})C(O)(C₁-C₆-alkyl), or —N(R^{C})C(O)(C₁-C₆-alkenyl). In some embodiments, A is —N(R^{C})—. In some embodiments, A is —N(R^{C})(R^{D}), and each R^{C} and R^{D} is independently hydrogen or alkyl. In some embodiments, A is —NH₂. In some embodiments, A is —N(R^{C})C(O)(C₁-C₆-alkyl), wherein alkyl is substituted with one or more R¹. In some embodiments, A is —N(R^{C})C(O)(C₁-C₆-alkenyl), wherein alkenyl is substituted with one or more R¹. In some embodiments, R¹ is C₁-C₆ alkyl (e.g., methyl). In some embodiments, A is —NHC(O)C(CH₃)(═CH₂). In some embodiments, A is —NH₂ or NHC(O)C(CH₃)(═CH₂).

In some embodiments, for Formulas (I), (I-a), and (I-b), L¹ is a bond, alkyl, or heteroalkyl. In some embodiments, L¹ is a bond or alkyl. In some embodiments, L¹ is a bond.

In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (I), (I-a), and (I-b), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I), (I-a), and (I-b), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂-)z, wherein z is an integer selected from 1 to 10. In some embodiments, z is an integer selected from 1 to 5. In some embodiments, M is —OCH₂CH₂—, (—OCH₂CH₂-)₂, (—OCH₂CH₂-)₃, (—OCH₂CH₂-)₄, or (—OCH₂CH₂-)₅. In some embodiments, M is —OCH₂CH₂—, (—OCH₂CH₂-)₂, (—OCH₂CH₂-)₃, or (—OCH₂CH₂-)₄. In some embodiments, M is (—OCH₂CH₂-)₃.

In some embodiments, for Formulas (I), (I-a), and (I-b), P is heteroaryl. In some embodiments, for Formulas (I), (I-a), and (I-b), P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is triazolyl. In some embodiments, P is 1,2,3-triazolyl. In some embodiments, P is.

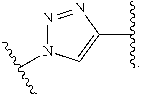 [SMILES: *c1cn(*)nn1]

In some embodiments, P is triazolyl substituted by one or more R⁴. In some embodiments, R⁴ is deuterium, alkyl, alkenyl, alkynyl, heteroalkyl, halogen, cyano, azido, —N(R^{C1})(R^{D1}), —N(R^{C1})C(O)R^{B1}, —C(O)N(R^{C1}), —S(O)ₓR^{E1}, —N(R^{C1})S(O)ₓR^{E1}, —S(O)ₓN(R^{C1})(R^{D1}), —P(R^{F1})_{y} cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷. In some embodiments, R⁴ is deuterium, alkyl, heteroalkyl, halogen, cyano, or azido, wherein each alkyl and heteroalkyl is optionally substituted by one or more R⁷ (e.g., halogen). In some embodiments, P is

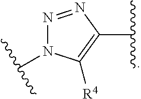

In some embodiments, P is triazolyl substituted by R⁴ (e.g., halogen). In some embodiments, R⁴ is deuterium, alkyl or halogen. In some embodiments, R⁴ is halogen (e.g., fluoro, chloro, bromo). In some embodiments, R⁴ is alkyl (e.g., —CH₃, —CH₂CH₃, —CF₃, —CH₂F, —CHF₂). In some embodiments, R⁴ is chloro. In some embodiments, P is

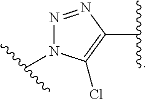 [SMILES: *c1nnn(*)c1Cl]

In some embodiments, P is

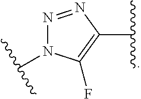 [SMILES: *c1nnn(*)c1F]

In some embodiments, P is

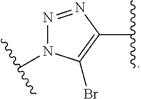 [SMILES: *c1nnn(*)c1Br]

In some embodiments, P is

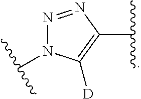 [SMILES: *c1nnn(*)c1[2H]]

In some embodiments, P is

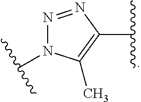 [SMILES: *c1nnn(*)c1C]

In some embodiments, P is

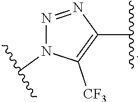 [SMILES: *c1nnn(*)c1C(F)(F)F]

In some embodiments, P is

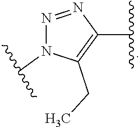 [SMILES: *c1nnn(*)c1CC]

In some embodiments, P is

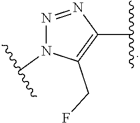 [SMILES: *c1nnn(*)c1CF]

In some embodiments, P is

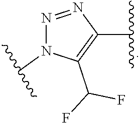 [SMILES: *c1nnn(*)c1C(F)F]

In some embodiments, for Formulas (I), (I-a), and (I-b), Z is heterocyclyl. In some embodiments, Z is monocyclic or bicyclic heterocyclyl. In some embodiments, Z is a 4-membered heterocyclyl, 5-membered heterocyclyl, or 6-membered heterocyclyl. In some embodiments, Z is a 4-membered heterocyclyl. In some embodiments, Z is a 6-membered heterocyclyl. In some embodiments, Z is a nitrogen-containing heterocyclyl. In some embodiments, Z is a sulfur-containing heterocyclyl. In some embodiments, Z is a 4-membered nitrogen-containing heterocyclyl. In some embodiments, Z is a 6-membered nitrogen-containing heterocyclyl. In some embodiments, Z is a 6-membered sulfur-containing heterocyclyl. In some embodiments, Z is a 6-membered heterocyclyl containing a nitrogen atom and a sulfur atom. In some embodiments, Z is a 4-membered nitrogen heterocyclyl optionally substituted with one R⁵ (e.g., —S(O)ₓR^{E1}). In some embodiments, R⁵ is —S(O)₂CH₃.

In some embodiments, Z is 3-(methylsulfonyl)azetidinyl. In some embodiments, Z is

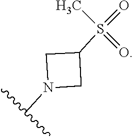 [SMILES: *N1CC(S(C)(=O)=O)C1]

In some embodiments, Z is thiomorpholinyl-1,1-dioxidyl. In some embodiments, Z is

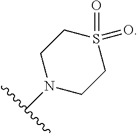 [SMILES: *N1CCS(=O)(=O)CC1]

In some embodiments, the compound of Formula (I) or (I-a) is a compound of Formula (I-c):

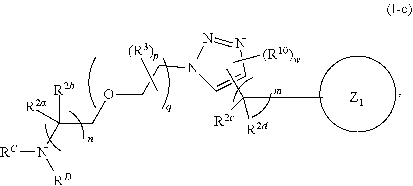

or a pharmaceutically acceptable salt thereof, wherein Ring Z¹ is heterocyclyl optionally substituted with 1-5 R⁵; each of R^{C} and R^{D} is independently hydrogen, alkyl, alkenyl, —C(O)(C₁-C₆-alkyl), or —C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen, alkyl, heteroalkyl, halo, or amino; or R^{2a} and R^{2b} or R^{2c} and R^{2d} are taken together to form an oxo group; each of R³, R⁵ and R⁶ is independently alkyl, heteroalkyl, halogen, oxo, —OR^{A1}, —C(O)OR^{A1}, —C(O)R^{B1}, —SR^{E1}, —S(O)ₓR^{E1}, or —OS(O)ₓR^{E1}; each R¹⁰ is independently alkyl, heteroalkyl, halo, cyano, nitro, or amino; each R^{A1}, R^{B1}, and R^{E1} is independently hydrogen, alkyl, or heteroalkyl; m and n are each independently 1, 2, 3, 4, 5, or 6; w is 0, 1, or 2; each of q and p is independently an integer from 0 to 25; and x is 0, 1, or 2.

In some embodiments, Ring Z¹ is heterocyclyl. In some embodiments, Ring Z¹ is nitrogen-containing heterocyclyl. In some embodiments, Ring Z¹ is 4-membered heterocyclyl or 6-membered heterocyclyl. In some embodiments, Ring Z¹ is heterocyclyl substituted with 1 R⁵. In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃).

In some embodiments, each of R^{2a}, R^{2b}, R^{2c}, and R^{2d} is independently hydrogen.

In some embodiments, each of R^{C} and R^{D} is independently hydrogen, —C(O)(C₁-C₆-alkyl), or —C(O)(C₁-C₆-alkenyl). In some embodiments, one of R^{C} and R^{D} is hydrogen and the other of R^{C} and R^{D} is —C(O)(C₁-C₆-alkyl), or —C(O)(C₁-C₆-alkenyl). In some embodiments, each of alkyl and alkenyl is substituted with 1 R⁶ (e.g., —CH3). In some embodiments, one of R^{C} and R^{D} is hydrogen and the other of R^{C} and R^{D} is —C(O)C(CH₃)(═CH₂).

In some embodiments, n is 1. In some embodiments, q is 2, 3, 4, or 5. In some embodiments, q is 3. In some embodiments, m is 1. In some embodiments, p is 0. In some embodiments, w is 0. In some embodiments, w is 1. In some embodiments, R¹⁰ is halo (e.g., Cl).

In some embodiments, the compound of Formula (I-c) is a compound of Formula (I-d):

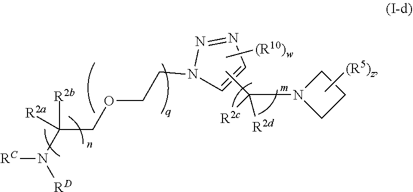

or a pharmaceutically acceptable salt thereof, wherein each of R^{C} and R^{D} is independently hydrogen, alkyl, —N(R^{c})C(O)R^{B}, —N(R^{c})C(O)(C₁-C₆-alkyl), or —N(R^{c})C(O)(C₁-C₆-alkenyl), wherein each of alkyl and alkenyl is optionally substituted with 1-6 R⁶; each of R^{2a}, R^{2b}, R^{2e}, and R^{2d} is independently hydrogen or alkyl; or R^{2a} and R^{2b} or R^{2e} and R^{2d} are taken together to form an oxo group; each of R⁵ and R⁶ is independently alkyl, heteroalkyl, halogen, oxo, —S(O)ₓR^{E1}, or —OS(O)ₓR^{E1}; each R¹⁰ is independently alkyl, heteroalkyl, halo, cyano, nitro, or amino; R^{E1} is independently hydrogen, alkyl, or heteroalkyl; m and n are each independently 1, 2, 3, 4, 5, or 6; w is 0, 1, or 2; q is an integer from 0 to 25; x is 0, 1, or 2; and z is 0, 1, 2, 3, 4, 5, or 6.

In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃). In some embodiments, z is 1.

In some embodiments, each of R^{2a}, R^{2b}, R^{2c}, and R^{2d} is independently hydrogen. In some embodiments, each of R^{C} and R^{D} is independently hydrogen, —C(O)(C₁-C₆-alkyl), or —C(O)(C₁-C₆-alkenyl). In some embodiments, each of R^{C} and R^{D} is hydrogen. In some embodiments, one of R^{C} and R^{D} is hydrogen and the other of R^{C} and R^{D} is —C(O)(C₁-C₆-alkyl), or —C(O)(C₁-C₆-alkenyl). In some embodiments, each of alkyl and alkenyl is substituted with 1 R⁶ (e.g., —CH3). In some embodiments, one of R^{C} and R^{D} is hydrogen and the other of R^{C} and R^{D} is —C(O)C(CH₃)(═CH₂).

In some embodiments, n is 1. In some embodiments, q is 2, 3, 4, or 5. In some embodiments, q is 3. In some embodiments, m is 1. In some embodiments, w is 0.

In some embodiments, the compound of Formula (I) is a compound of Formula (I-e):

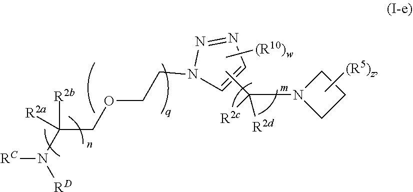

or a pharmaceutically acceptable salt thereof, wherein each of R^{C} and R^{D} is independently hydrogen, alkyl, —N(R^{c})C(O)R^{B}, —N(R^{c})C(O)(C₁-C₆-alkyl), or —N(R^{c})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2e} and R^{2d} are taken together to form an oxo group; each of R⁵ and R⁶ is independently alkyl, heteroalkyl, halogen, oxo, —S(O)ₓR^{E1}, or —OS(O)ₓR^{E1}; each R¹⁰ is independently deuterium, alkyl, heteroalkyl, halo, cyano, azido, nitro, or amino, wherein each alkyl or heteroalkyl is optionally substituted by one or more R¹¹; each R¹¹ is independently alkyl, alkenyl, alkynyl, heteroalkyl, halogen, cyano, oxo, hydroxyl, cycloalkyl, or heterocyclyl; R^{E1} is independently hydrogen, alkyl, or heteroalkyl; m and n are each independently 1, 2, 3, 4, 5, or 6; w is 0, 1, or 2; q is an integer from 0 to 25; x is 0, 1, or 2; and z is 0, 1, 2, 3, 4, 5, or 6.

In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃). In some embodiments, z is 1.

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen). In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, each of R^{C} and R^{D} is independently hydrogen, —C(O)(C₁-C₆-alkyl), or —C(O)(C₁-C₆-alkenyl). In some embodiments, each of R^{C} and R^{D} is hydrogen. In some embodiments, one of R^{C} and R^{D} is hydrogen and the other of R^{C} and R^{D} is —C(O)(C₁-C₆-alkyl), or —C(O)(C₁-C₆-alkenyl). In some embodiments, each of alkyl and alkenyl is substituted with 1 R⁶ (e.g., —CH3). In some embodiments, one of R^{C} and R^{D} is hydrogen and the other of R^{C} and R^{D} is —C(O)C(CH₃)(═CH₂).

In some embodiments, n is 1. In some embodiments, q is 2, 3, 4, or 5. In some embodiments, q is 3. In some embodiments, m is 1. In some embodiments, w is 0.

In some embodiments, the compound of Formula (I-d) is a compound of Formula (I-f):

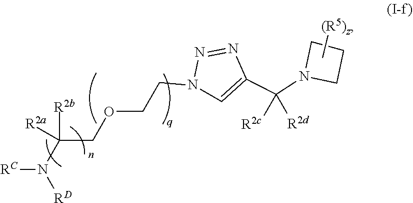

or a pharmaceutically acceptable salt thereof, wherein each of R^{C} and R^{D} is independently hydrogen, alkyl, —N(R^{c})C(O)R^{B}, —N(R^{c})C(O)(C₁-C₆-alkyl), or —N(R^{c})C(O)(C₁-C₆-alkenyl), wherein each of alkyl and alkenyl is optionally substituted with 1-6 R⁶; each of R^{2a}, R^{2b}, R^{2e}, and R^{2d} is independently hydrogen or alkyl; or R^{2a} and R^{2b} or R^{2e} and R^{2d} are taken together to form an oxo group; each of R⁵ and R⁶ is independently alkyl, heteroalkyl, halogen, oxo, —S(O)ₓR^{E1}, or —OS(O)ₓR^{E1}; R^{E1} is independently hydrogen, alkyl, or heteroalkyl; n is 1, 2, 3, 4, 5, or 6; q is an integer from 0 to 25; x is 0, 1, or 2; and z is 0, 1, 2, 3, 4, 5, or 6.

In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃). In some embodiments, z is 1.

In some embodiments, each of R^{2a}, R^{2b}, R^{2c}, and R^{2d} is independently hydrogen.

In some embodiments, each of R^{C} and R^{D} is independently hydrogen, —C(O)(C₁-C₆-alkyl), or —C(O)(C₁-C₆-alkenyl). In some embodiments, each of R^{C} and R^{D} is hydrogen. In some embodiments, one of R^{C} and R^{D} is hydrogen and the other of R^{C} and R^{D} is —C(O)(C₁-C₆-alkyl), or —C(O)(C₁-C₆-alkenyl). In some embodiments, each of alkyl and alkenyl is substituted with 1 R⁶ (e.g., —CH3). In some embodiments, one of R^{C} and R^{D} is hydrogen and the other of R^{C} and R^{D} is —C(O)C(CH₃)(═CH₂).

In some embodiments, n is 1. In some embodiments, q is 2, 3, 4, or 5. In some embodiments, q is 3. In some embodiments, m is 1.

In some embodiments, the compound of Formula (I-c) is a compound of Formula (I-g):

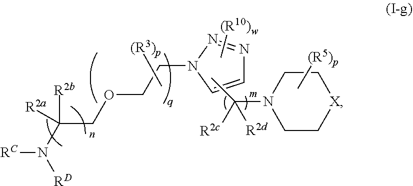

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2e} and R^{2d} are taken together to form an oxo group; each of R^{C} and R^{D} is independently hydrogen, alkyl, —N(R^{C})C(O)R^{B}, —N(R^{c})C(O)(C₁-C₆-alkyl), or —N(R^{c})C(O)(C₁-C₆-alkenyl), wherein each of alkyl and alkenyl is optionally substituted with 1-6 R⁶; each of R³, R⁵, and R⁶ is independently alkyl, heteroalkyl, halogen, oxo, —OR^{A1}, —C(O)OR^{A1}, —C(O)R^{B1}, —SR^{E1}, —S(O)ₓR^{E1}, or —OS(O)ₓR^{E1}; each R¹⁰ is independently alkyl, heteroalkyl, halo, cyano, nitro, or amino; each R^{A1}, R^{B1}, and R^{E1} is independently hydrogen, alkyl, or heteroalkyl; m and n are each independently 1, 2, 3, 4, 5, or 6; w is 1; each of q and p is independently an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, each of R^{C} and R^{D} is independently hydrogen, —C(O)(C₁-C₆-alkyl), or —C(O)(C₁-C₆-alkenyl). In some embodiments, each of R^{C} and R^{D} is hydrogen. In some embodiments, one of R^{C} and R^{D} is hydrogen and the other of R^{C} and R^{D} is —C(O)(C₁-C₆-alkyl), or —C(O)(C₁-C₆-alkenyl). In some embodiments, each of alkyl and alkenyl is substituted with 1 R⁶ (e.g., —CH₃). In some embodiments, one of R^{C} and R^{D} is hydrogen and the other of R^{C} and R^{D} is —C(O)C(CH₃)(═CH₂).

In some embodiments, n is 1. In some embodiments, q is 2, 3, 4, or 5. In some embodiments, q is 3. In some embodiments, m is 1. In some embodiments, p is 0. In some embodiments, R¹⁰ is halo (e.g., Cl).

In some embodiments, the compound of Formula (I-g) is a compound of Formula (I-h):

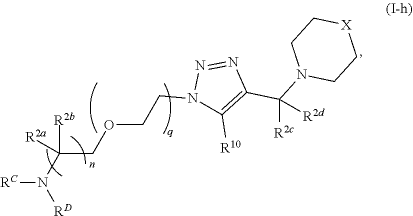

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2e} and R^{2d} are taken together to form an oxo group; each of R^{C} and R^{D} is independently hydrogen, alkyl, —N(R^{C})C(O)R^{B}, —N(R^{c})C(O)(C₁-C₆-alkyl), or —N(R^{c})C(O)(C₁-C₆-alkenyl), wherein each of alkyl and alkenyl is optionally substituted with 1-6 R⁶; each of R⁶ is independently alkyl, heteroalkyl, halogen, oxo, —OR^{A1}, —C(O)OR^{A1}, —C(O)R^{B1}; R¹⁰ is independently alkyl, heteroalkyl, halo, cyano, nitro, or amino; each R^{A1} and R^{B1} is independently hydrogen, alkyl, or heteroalkyl; n is 1, 2, 3, 4, 5, or 6; q is an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, each of R^{C} and R^{D} is independently hydrogen, —C(O)(C₁-C₆-alkyl), or —C(O)(C₁-C₆-alkenyl). In some embodiments, each of R^{C} and R^{D} is hydrogen. In some embodiments, one of R^{C} and R^{D} is hydrogen and the other of R^{C} and R^{D} is —C(O)(C₁-C₆-alkyl), or —C(O)(C₁-C₆-alkenyl). In some embodiments, each of alkyl and alkenyl is substituted with 1 R⁶ (e.g., —CH₃). In some embodiments, one of R^{C} and R^{D} is hydrogen and the other of R^{C} and R^{D} is —C(O)C(CH₃)(═CH₂).

In some embodiments, n is 1. In some embodiments, q is 3. In some embodiments, m is 1. In some embodiments, R¹⁰ is halo (e.g., Cl).

In some embodiments, the compound of Formula (I) is a compound of Formula (I-i):

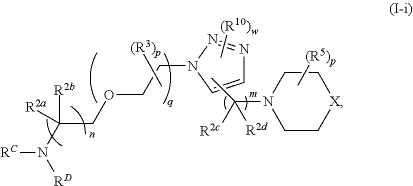

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2e} and R^{2d} are taken together to form an oxo group; each of R^{C} and R^{D} is independently hydrogen, alkyl, —N(R^{C})C(O)R^{B}, —N(R^{c})C(O)(C₁-C₆-alkyl), or —N(R^{c})C(O)(C₁-C₆-alkenyl), wherein each of alkyl and alkenyl is optionally substituted with 1-6 R⁶; each of R³, R⁵, and R⁶ is independently alkyl, heteroalkyl, halogen, oxo, —OR^{A1}, —C(O)OR^{A1}, —C(O)R^{B1}, —SR^{E1}, —S(O)ₓR^{E1}, or —OS(O)ₓR^{E1}; each R¹⁰ is independently deuterium, alkyl, heteroalkyl, halo, cyano, azido, nitro, or amino, wherein each alkyl or heteroalkyl is optionally substituted by one or more R¹¹; each R¹¹ is independently alkyl, alkenyl, alkynyl, heteroalkyl, halogen, cyano, oxo, hydroxyl, cycloalkyl, or heterocyclyl; each R^{A1}, R^{B1}, and R^{E1} is independently hydrogen, alkyl, or heteroalkyl; m and n are each independently 1, 2, 3, 4, 5, or 6; w is 1; each of q and p is independently an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen).

In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, each of R^{C} and R^{D} is independently hydrogen, —C(O)(C₁-C₆-alkyl), or —C(O)(C₁-C₆-alkenyl). In some embodiments, each of R^{C} and R^{D} is hydrogen. In some embodiments, one of R^{C} and R^{D} is hydrogen and the other of R^{C} and R^{D} is —C(O)(C₁-C₆-alkyl), or —C(O)(C₁-C₆-alkenyl). In some embodiments, each of alkyl and alkenyl is substituted with 1 R⁶ (e.g., —CH₃). In some embodiments, one of R^{C} and R^{D} is hydrogen and the other of R^{C} and R^{D} is —C(O)C(CH₃)(═CH₂).

In some embodiments, n is 1. In some embodiments, q is 2, 3, 4, or 5. In some embodiments, q is 3. In some embodiments, m is 1. In some embodiments, p is 0.

In some embodiments, the compound of Formula (I-i) is a compound of Formula (I-j):

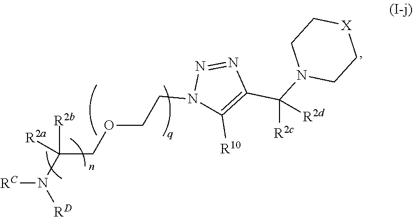

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2e} and R^{2d} are taken together to form an oxo group; each of R^{C} and R^{D} is independently hydrogen, alkyl, —N(R^{C})C(O)R^{B}, —N(R^{c})C(O)(C₁-C₆-alkyl), or —N(R^{c})C(O)(C₁-C₆-alkenyl), wherein each of alkyl and alkenyl is optionally substituted with 1-6 R⁶; each of R⁶ is independently alkyl, heteroalkyl, halogen, oxo, —OR^{A1}, —C(O)OR^{A1}, —C(O)R^{B1}; R¹⁰ is deuterium, alkyl, heteroalkyl, halo, cyano, azido, nitro, or amino, wherein each alkyl or heteroalkyl is optionally substituted by one or more R″; each R″ is independently alkyl, alkenyl, alkynyl, heteroalkyl, halogen, cyano, oxo, hydroxyl, cycloalkyl, or heterocyclyl; each R^{A1} and R^{B1} is independently hydrogen, alkyl, or heteroalkyl; n is 1, 2, 3, 4, 5, or 6; q is an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen).

In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, each of R^{C} and R^{D} is independently hydrogen, —C(O)(C₁-C₆-alkyl), or —C(O)(C₁-C₆-alkenyl). In some embodiments, each of R^{C} and R^{D} is hydrogen. In some embodiments, one of R^{C} and R^{D} is hydrogen and the other of R^{C} and R^{D} is —C(O)(C₁-C₆-alkyl), or —C(O)(C₁-C₆-alkenyl). In some embodiments, each of alkyl and alkenyl is substituted with 1 R⁶ (e.g., —CH₃). In some embodiments, one of R^{C} and R^{D} is hydrogen and the other of R^{C} and R^{D} is —C(O)C(CH₃)(═CH₂).

In some embodiments, n is 1. In some embodiments, q is 3. In some embodiments, m is 1.

In some embodiments, the compound of Formula (I) is a compound of Formula (I-k):

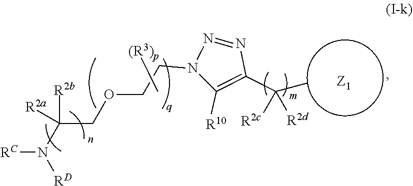

or a pharmaceutically acceptable salt thereof, wherein Ring Z¹ is heterocyclyl optionally substituted with 1-5 R⁵; each of R^{C} and R^{D} is independently hydrogen, alkyl, alkenyl, —C(O)(C₁-C₆-alkyl), or —C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen, alkyl, heteroalkyl, halo, or amino; or R^{2a} and R^{2b} or R^{2e} and R^{2d} are taken together to form an oxo group; each of R³, R⁵ and R⁶ is independently alkyl, heteroalkyl, halogen, oxo, —OR^{A1}, —C(O)OR^{A1}, —C(O)R^{B1}, —SR^{E1}, S(O)ₓR^{E1}, or —OS(O)ₓR^{E1}; R¹⁰ is hydrogen, deuterium, alkyl, heteroalkyl, halo, cyano, azido, nitro, or amino, wherein each alkyl or heteroalkyl is optionally substituted by one or more R¹¹; each R¹¹ is independently alkyl, alkenyl, alkynyl, heteroalkyl, halogen, cyano, oxo, hydroxyl, cycloalkyl, or heterocyclyl; each R^{A1}, R^{B1}, and R^{E1} is independently hydrogen, alkyl, or heteroalkyl; m and n are each independently 1, 2, 3, 4, 5, or 6; w is 0, 1, or 2; each of q and p is independently an integer from 0 to 25; and x is 0, 1, or 2.

In some embodiments, Ring Z¹ is heterocyclyl. In some embodiments, Ring Z¹ is nitrogen-containing heterocyclyl. In some embodiments, Ring Z¹ is 4-membered heterocyclyl or 6-membered heterocyclyl. In some embodiments, Ring Z¹ is heterocyclyl substituted with 1 R⁵. In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃).

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen). In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, each of R^{C} and R^{D} is independently hydrogen, —C(O)(C₁-C₆-alkyl), or —C(O)(C₁-C₆-alkenyl). In some embodiments, one of R^{C} and R^{D} is hydrogen and the other of R^{C} and R^{D} is —C(O)(C₁-C₆-alkyl), or —C(O)(C₁-C₆-alkenyl). In some embodiments, each of alkyl and alkenyl is substituted with 1 R⁶ (e.g., —CH3). In some embodiments, one of R^{C} and R^{D} is hydrogen and the other of R^{C} and R^{D} is —C(O)C(CH₃)(═CH₂).

In some embodiments, n is 1. In some embodiments, q is 2, 3, 4, or 5. In some embodiments, q is 3. In some embodiments, m is 1. In some embodiments, p is 0. In some embodiments, w is 0. In some embodiments, w is 1. In some embodiments, R¹⁰ is halo (e.g., Cl).

In some embodiments, the compound of Formula (I) is a compound of Formula (I-1):

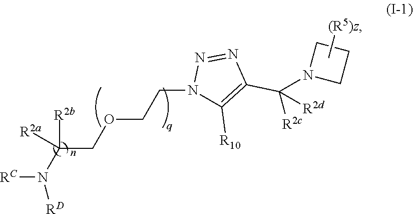

or a pharmaceutically acceptable salt thereof, wherein each of R^{C} and R^{D} is independently hydrogen, alkyl, —N(R^{c})C(O)R^{B}, —N(R^{c})C(O)(C₁-C₆-alkyl), or —N(R^{c})C(O)(C₁-C₆-alkenyl), wherein each of alkyl and alkenyl is optionally substituted with 1-6 R⁶; each of R^{2a}, R^{2b}, R^{2e}, and R^{2d} is independently hydrogen or alkyl; or R^{2a} and R^{2b} or R^{2e} and R^{2d} are taken together to form an oxo group; each of R⁵ and R⁶ is independently alkyl, heteroalkyl, halogen, oxo, —S(O)ₓR^{E1}, or —OS(O)ₓR^{E1}; R¹⁰ is deuterium, alkyl, heteroalkyl, halo, cyano, azido, nitro, or amino, wherein each alkyl or heteroalkyl is optionally substituted by one or more R¹¹; each R¹¹ is independently alkyl, alkenyl, alkynyl, heteroalkyl, halogen, cyano, oxo, hydroxyl, cycloalkyl, or heterocyclyl; R^{E1} is independently hydrogen, alkyl, or heteroalkyl; n is 1, 2, 3, 4, 5, or 6; q is an integer from 0 to 25; x is 0, 1, or 2; and z is 0, 1, 2, 3, 4, 5, or 6.

In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃). In some embodiments, z is 1.

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen). In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, each of R^{C} and R^{D} is independently hydrogen, —C(O)(C₁-C₆-alkyl), or —C(O)(C₁-C₆-alkenyl). In some embodiments, each of R^{C} and R^{D} is hydrogen. In some embodiments, one of R^{C} and R^{D} is hydrogen and the other of R^{C} and R^{D} is —C(O)(C₁-C₆-alkyl), or —C(O)(C₁-C₆-alkenyl). In some embodiments, each of alkyl and alkenyl is substituted with 1 R⁶ (e.g., —CH3). In some embodiments, one of R^{C} and R^{D} is hydrogen and the other of R^{C} and R^{D} is —C(O)C(CH₃)(═CH₂).

In some embodiments, n is 1. In some embodiments, q is 2, 3, 4, or 5. In some embodiments, q is 3. In some embodiments, m is 1.

In some embodiments, the compound is a compound of Formula (I), (I-a), (I-b), (I-c), (I-d), or a pharmaceutically acceptable salt thereof. In an embodiment, the compound is Compound 300 or 302.

In some embodiments, the compound is a compound of Formula (I), (I-a), (I-b), (I-e), (I-f), or a pharmaceutically acceptable salt thereof. In an embodiment, the compound is Compound 301 or 303.

In some embodiments, the compound of Formula (I) (e.g., Formula (I-a), (I-b), (I-c), (I-d), (I-e), (I-f), (I-g), (I-h), (I-i), (I-j), (I-k), (I-l)), or a pharmaceutically acceptable salt thereof is not a compound disclosed in WO2012/112982, WO2012/167223, WO2014/153126, WO2016/187225, WO2016/019391, WO2017/075630, WO 2017/075631, WO 2018/067615, WO 2019/169333, or US 2016-0030359.

TABLE 1
Exemplary compounds of Formula (I)

| Compound No. | Structure |
|---|---|
| 300 | 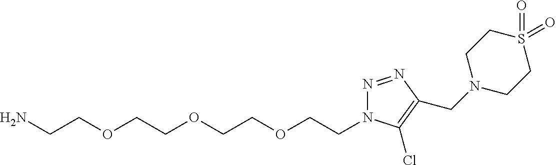 [SMILES: NCCOCCOCCOCCn1nnc(CN2CCS(=O)(=O)CC2)c1Cl] |
| 301 | 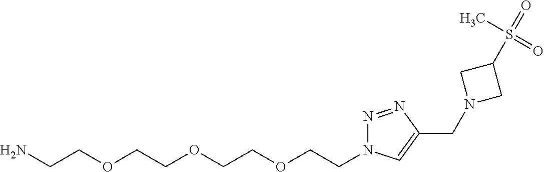 [SMILES: CS(=O)(=O)C1CN(Cc2cn(CCOCCOCCOCCN)nn2)C1] |
| 302 | 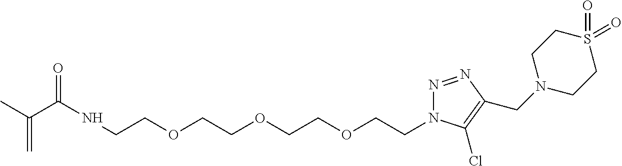 [SMILES: C=C(C)C(=O)NCCOCCOCCOCCn1nnc(CN2CCS(=O)(=O)CC2)c1Cl] |
| 303 | 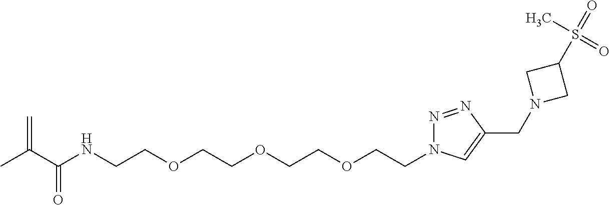 [SMILES: C=C(C)C(=O)NCCOCCOCCOCCn1cc(CN2CC(S(C)(=O)=O)C2)nn1] |
| 304 | 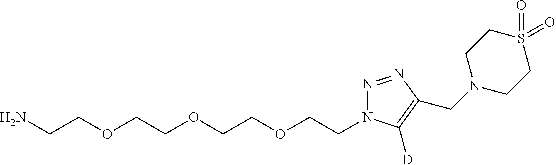 [SMILES: [2H]c1c(CN2CCS(=O)(=O)CC2)nnn1CCOCCOCCOCCN] |
| 305 | 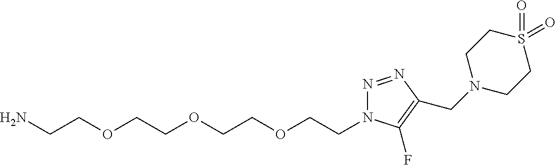 [SMILES: NCCOCCOCCOCCn1nnc(CN2CCS(=O)(=O)CC2)c1F] |
| 306 | 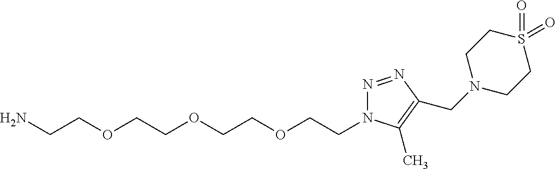 [SMILES: Cc1c(CN2CCS(=O)(=O)CC2)nnn1CCOCCOCCOCCN] |
| 307 | 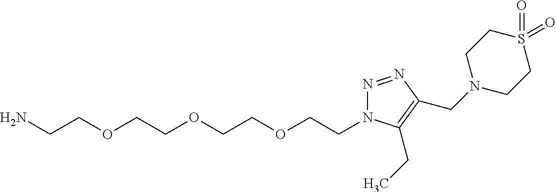 [SMILES: CCc1c(CN2CCS(=O)(=O)CC2)nnn1CCOCCOCCOCCN] |
| 308 | 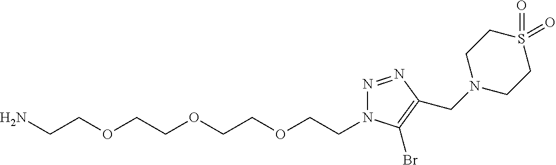 [SMILES: NCCOCCOCCOCCn1nnc(CN2CCS(=O)(=O)CC2)c1Br] |
| 309 | 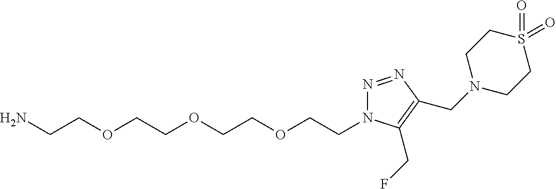 [SMILES: NCCOCCOCCOCCn1nnc(CN2CCS(=O)(=O)CC2)c1CF] |
| 310 | 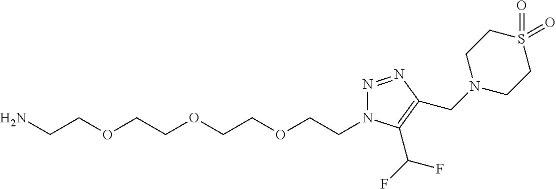 [SMILES: NCCOCCOCCOCCn1nnc(CN2CCS(=O)(=O)CC2)c1C(F)F] |
| 311 | 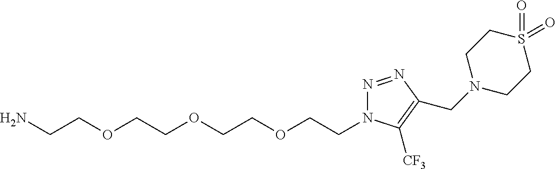 [SMILES: NCCOCCOCCOCCn1nnc(CN2CCS(=O)(=O)CC2)c1C(F)(F)F] |
| 312 | 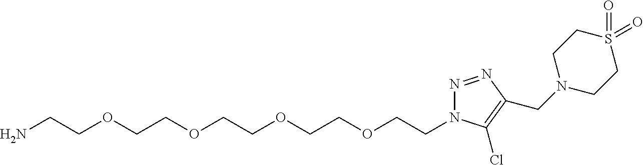 [SMILES: NCCOCCOCCOCCOCCn1nnc(CN2CCS(=O)(=O)CC2)c1Cl] |
| 313 | 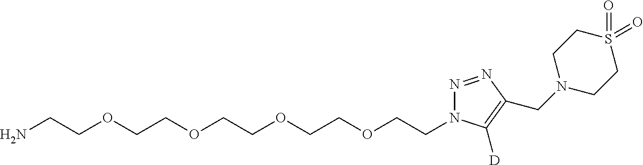 [SMILES: [2H]c1c(CN2CCS(=O)(=O)CC2)nnn1CCOCCOCCOCCOCCN] |
| 314 | 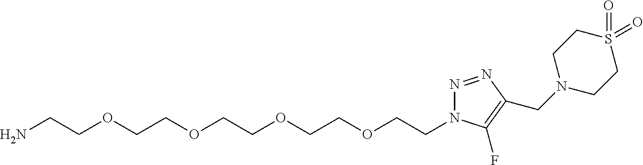 [SMILES: NCCOCCOCCOCCOCCn1nnc(CN2CCS(=O)(=O)CC2)c1F] |
| 315 | 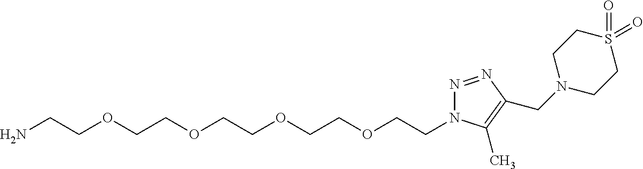 [SMILES: Cc1c(CN2CCS(=O)(=O)CC2)nnn1CCOCCOCCOCCOCCN] |
| 316 | 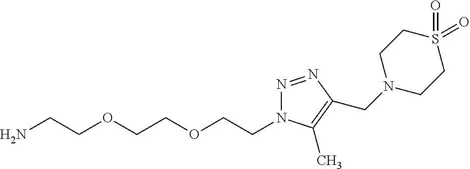 [SMILES: Cc1c(CN2CCS(=O)(=O)CC2)nnn1CCOCCOCCN] |
| 317 | 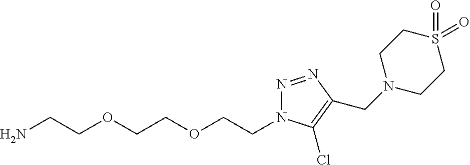 [SMILES: NCCOCCOCCn1nnc(CN2CCS(=O)(=O)CC2)c1Cl] |
| 318 | 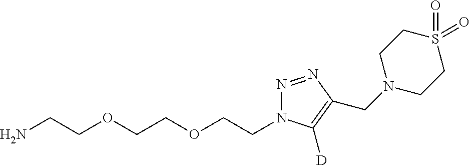 [SMILES: [2H]c1c(CN2CCS(=O)(=O)CC2)nnn1CCOCCOCCN] |
| 319 | 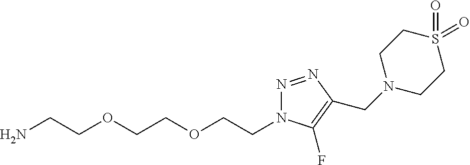 [SMILES: NCCOCCOCCn1nnc(CN2CCS(=O)(=O)CC2)c1F] |
| 320 | 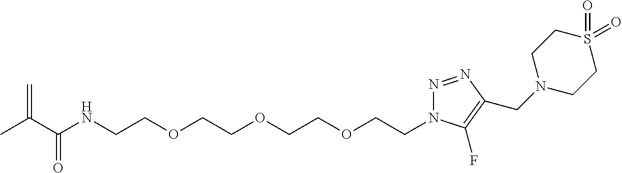 [SMILES: C=C(C)C(=O)NCCOCCOCCOCCn1nnc(CN2CCS(=O)(=O)CC2)c1F] |
| 321 | 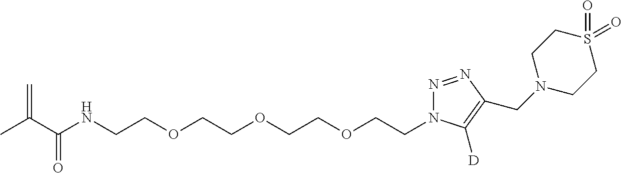 [SMILES: [2H]c1c(CN2CCS(=O)(=O)CC2)nnn1CCOCCOCCOCCNC(=O)C(=C)C] |
| 322 | 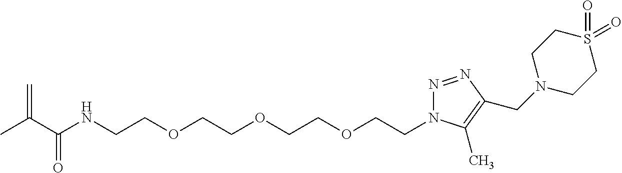 [SMILES: C=C(C)C(=O)NCCOCCOCCOCCn1nnc(CN2CCS(=O)(=O)CC2)c1C] |
| 323 | 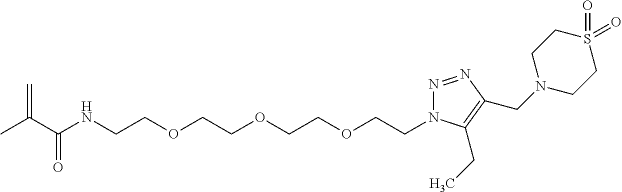 [SMILES: C=C(C)C(=O)NCCOCCOCCOCCn1nnc(CN2CCS(=O)(=O)CC2)c1CC] |
| 324 | 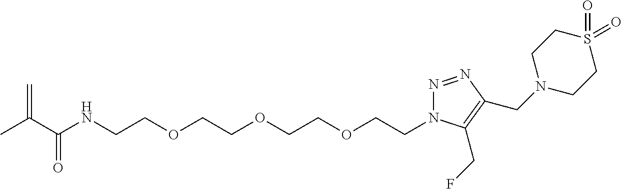 [SMILES: C=C(C)C(=O)NCCOCCOCCOCCn1nnc(CN2CCS(=O)(=O)CC2)c1CF] |
| 325 | 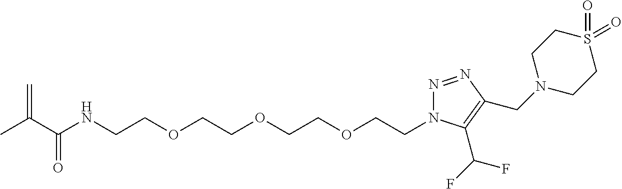 [SMILES: C=C(C)C(=O)NCCOCCOCCOCCn1nnc(CN2CCS(=O)(=O)CC2)c1C(F)F] |
| 326 | 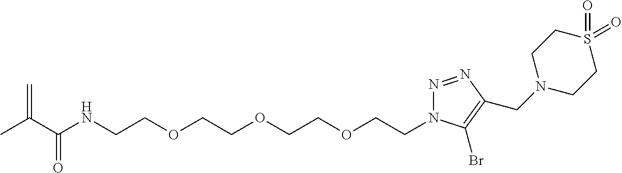 [SMILES: C=C(C)C(=O)NCCOCCOCCOCCn1nnc(CN2CCS(=O)(=O)CC2)c1Br] |
| 327 | 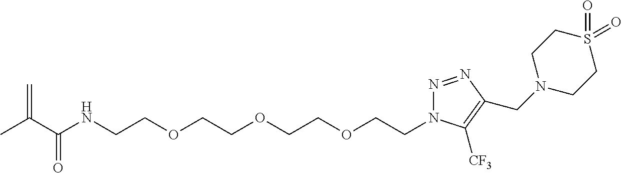 [SMILES: C=C(C)C(=O)NCCOCCOCCOCCn1nnc(CN2CCS(=O)(=O)CC2)c1C(F)(F)F] |
| 328 | 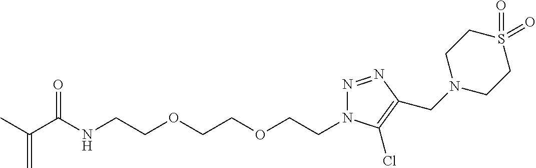 [SMILES: C=C(C)C(=O)NCCOCCOCCn1nnc(CN2CCS(=O)(=O)CC2)c1Cl] |
| 329 | 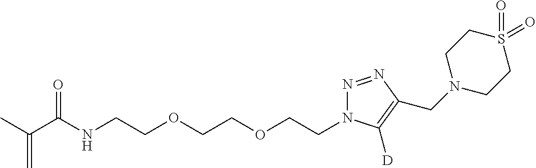 [SMILES: [2H]c1c(CN2CCS(=O)(=O)CC2)nnn1CCOCCOCCNC(=O)C(=C)C] |
| 330 | 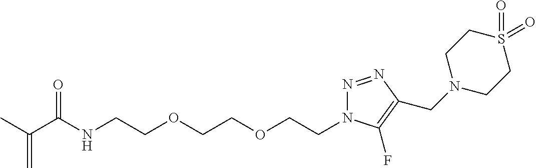 [SMILES: C=C(C)C(=O)NCCOCCOCCn1nnc(CN2CCS(=O)(=O)CC2)c1F] |
| 331 | 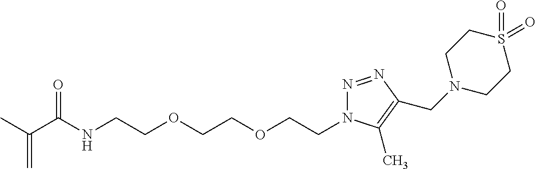 [SMILES: C=C(C)C(=O)NCCOCCOCCn1nnc(CN2CCS(=O)(=O)CC2)c1C] |
| 332 | 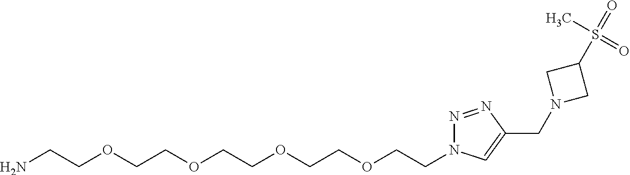 [SMILES: CS(=O)(=O)C1CN(Cc2cn(CCOCCOCCOCCOCCN)nn2)C1] |
| 333 | 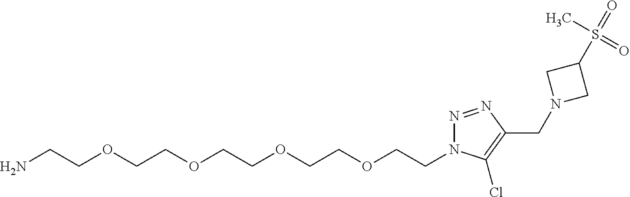 [SMILES: CS(=O)(=O)C1CN(Cc2nnn(CCOCCOCCOCCOCCN)c2Cl)C1] |
| 334 | 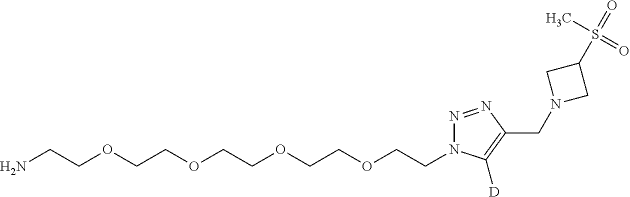 [SMILES: [2H]c1c(CN2CC(S(C)(=O)=O)C2)nnn1CCOCCOCCOCCOCCN] |
| 335 | 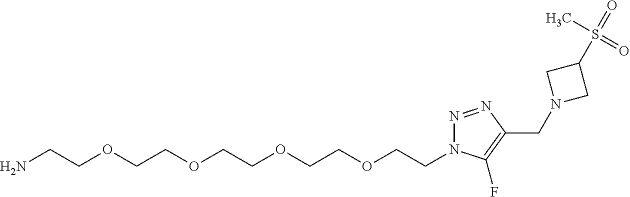 [SMILES: CS(=O)(=O)C1CN(Cc2nnn(CCOCCOCCOCCOCCN)c2F)C1] |
| 336 | 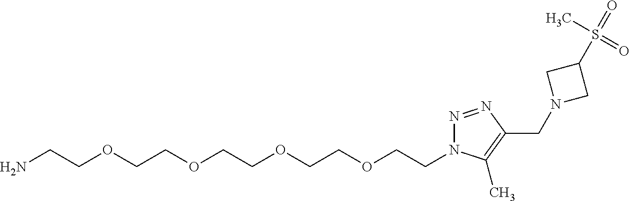 [SMILES: Cc1c(CN2CC(S(C)(=O)=O)C2)nnn1CCOCCOCCOCCOCCN] |
| 337 | 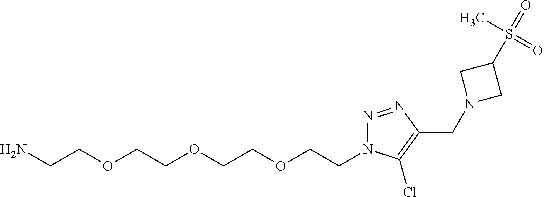 [SMILES: CS(=O)(=O)C1CN(Cc2nnn(CCOCCOCCOCCN)c2Cl)C1] |
| 338 | 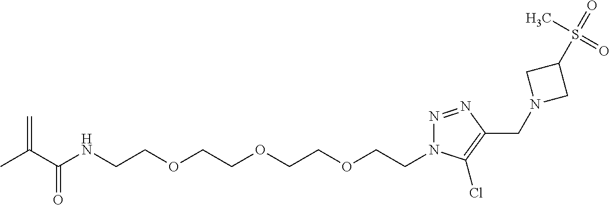 [SMILES: C=C(C)C(=O)NCCOCCOCCOCCn1nnc(CN2CC(S(C)(=O)=O)C2)c1Cl] |
| 339 | 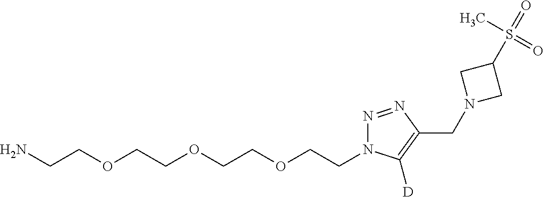 [SMILES: [2H]c1c(CN2CC(S(C)(=O)=O)C2)nnn1CCOCCOCCOCCN] |
| 340 | 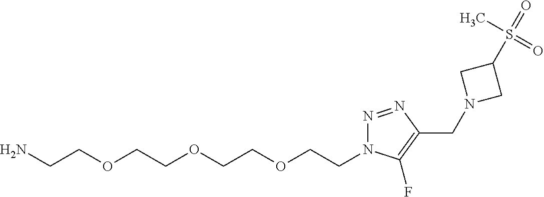 [SMILES: CS(=O)(=O)C1CN(Cc2nnn(CCOCCOCCOCCN)c2F)C1] |
| 341 | 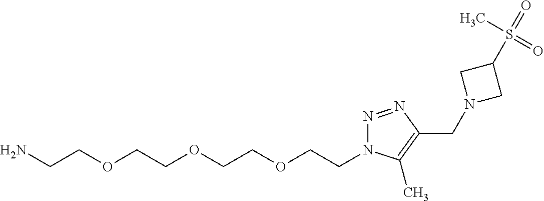 [SMILES: Cc1c(CN2CC(S(C)(=O)=O)C2)nnn1CCOCCOCCOCCN] |
| 342 | 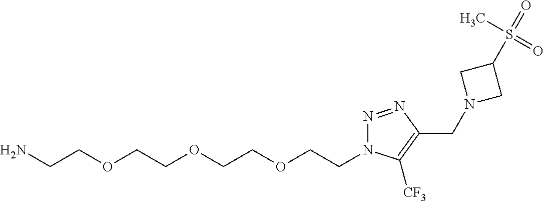 [SMILES: CS(=O)(=O)C1CN(Cc2nnn(CCOCCOCCOCCN)c2C(F)(F)F)C1] |
| 343 | 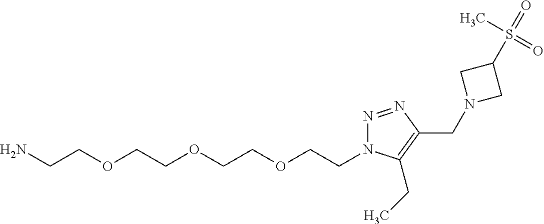 [SMILES: CCc1c(CN2CC(S(C)(=O)=O)C2)nnn1CCOCCOCCOCCN] |
| 344 | 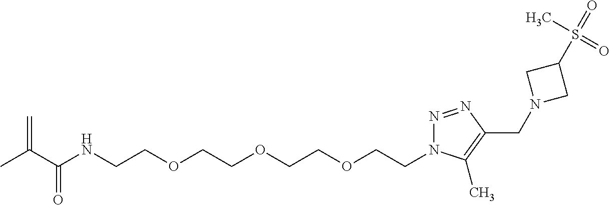 [SMILES: C=C(C)C(=O)NCCOCCOCCOCCn1nnc(CN2CC(S(C)(=O)=O)C2)c1C] |
| 345 | 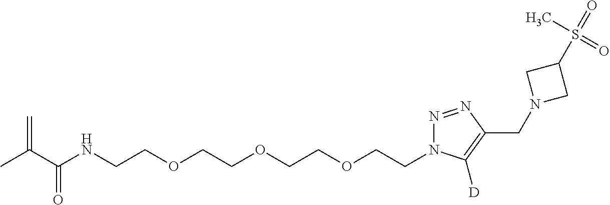 [SMILES: [2H]c1c(CN2CC(S(C)(=O)=O)C2)nnn1CCOCCOCCOCCNC(=O)C(=C)C] |
| 346 | 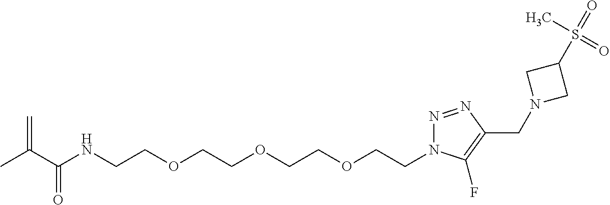 [SMILES: C=C(C)C(=O)NCCOCCOCCOCCn1nnc(CN2CC(S(C)(=O)=O)C2)c1F] |
| 347 | 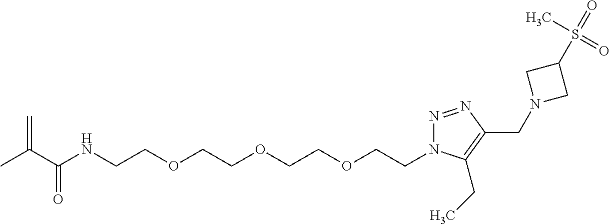 [SMILES: C=C(C)C(=O)NCCOCCOCCOCCn1nnc(CN2CC(S(C)(=O)=O)C2)c1CC] |
| 348 | 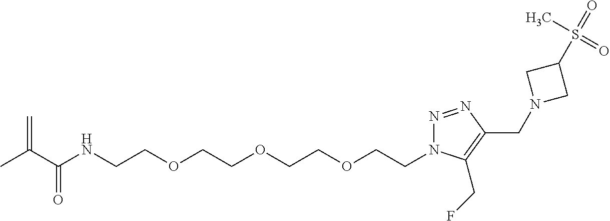 [SMILES: C=C(C)C(=O)NCCOCCOCCOCCn1nnc(CN2CC(S(C)(=O)=O)C2)c1CF] |
| 349 | 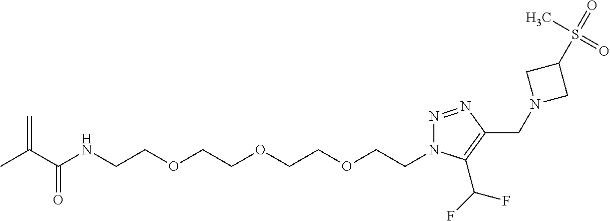 [SMILES: C=C(C)C(=O)NCCOCCOCCOCCn1nnc(CN2CC(S(C)(=O)=O)C2)c1C(F)F] |
| 350 | 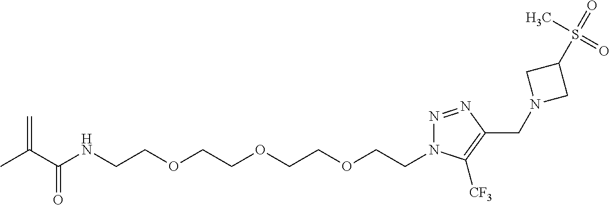 [SMILES: C=C(C)C(=O)NCCOCCOCCOCCn1nnc(CN2CC(S(C)(=O)=O)C2)c1C(F)(F)F] |
| 351 | 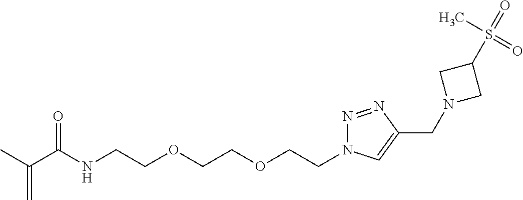 [SMILES: C=C(C)C(=O)NCCOCCOCCn1cc(CN2CC(S(C)(=O)=O)C2)nn1] |
| 352 | 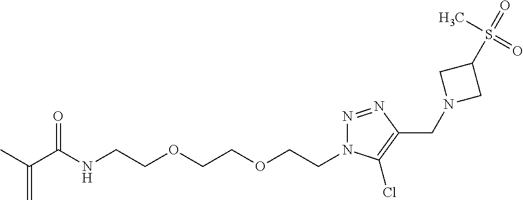 [SMILES: C=C(C)C(=O)NCCOCCOCCn1nnc(CN2CC(S(C)(=O)=O)C2)c1Cl] |
| 353 | 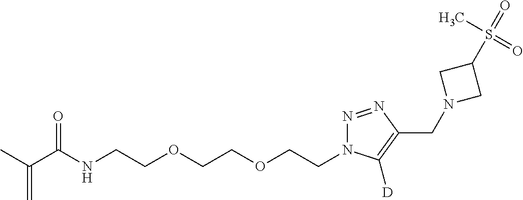 [SMILES: [2H]c1c(CN2CC(S(C)(=O)=O)C2)nnn1CCOCCOCCNC(=O)C(=C)C] |
| 354 | 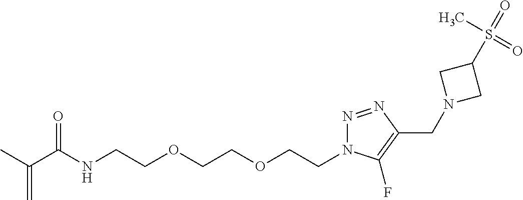 [SMILES: C=C(C)C(=O)NCCOCCOCCn1nnc(CN2CC(S(C)(=O)=O)C2)c1F] |
| 355 | 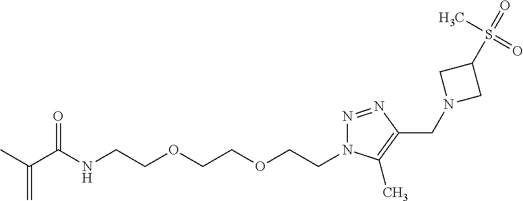 [SMILES: C=C(C)C(=O)NCCOCCOCCn1nnc(CN2CC(S(C)(=O)=O)C2)c1C] |

Compounds of Formula (II)

The disclosure features polymers modified with, and implantable elements comprising, a compound of Formula (II):

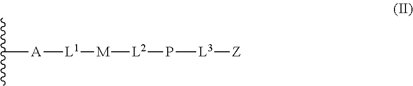

or a pharmaceutically acceptable salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{c})C(O)(C₁-C₆-alkenylene)-, —N(R^{C})N(R^{D})—, —NCN—, —C(═N(R^{C})(R^{D}))O—, —S—, —S(O), —OS(O)ₓ, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is absent, cycloalkyl, heterocycyl, or heteroaryl, each of which is optionally substituted by one or more R⁴;

Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R6;

each R¹, R², R³, R⁴, R⁵, and R⁶ is independently deuterium, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷; each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, the present invention features a compound of Formula (II-a):

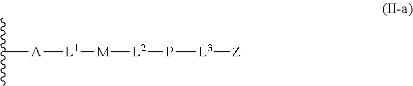

or a pharmaceutically acceptable salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ, —OS(O)ₓ, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is absent, cycloalkyl, heterocycyl, or heteroaryl, each of which is optionally substituted by one or more R⁴;

Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷; each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, the compound of Formula (II) or (II-a) is a compound of Formula (II-b):

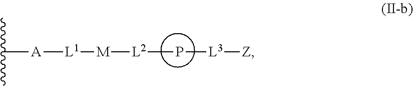

or a pharmaceutically acceptable salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)ₓ—, —OS(O)ₓ, —N(R^{C})S(O)ₓ, —S(O)ₓN(R^{C})—, —P(R^{F})_{y}, —Si(OR^{A})₂—, —Si(R^{G})(OR^{A})—, —B(OR^{A}), or a metal, each of which is optionally linked to an attachment group (e.g., an attachment group described herein) and optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is heteroaryl optionally substituted by one or more R⁴;

Z is alkyl, alkenyl, alkynyl, heteroalkyl, cycloalkyl, heterocyclyl, aryl, or heteroaryl, each of which is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;

each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, for Formulas (II), (II-a), and (II-b), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₁-C₆-alkenylene)-, or —N(R^{C})—. In some embodiments, A is alkyl, N(R^{C})C(O)—, —N(R^{C})C(O)(C₁-C₆-alkylene)-, —N(R^{C})C(O)(C₁-C₆-alkenylene)-, or —N(R^{C})—. In some embodiments, A is alkyl. In some embodiments, A is —N(R^{C})—. In some embodiments, A is —N(R^{c})—, and R^{C} an R^{D} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and R¹ is alkyl (e.g., methyl). In some embodiments, A is —NHC(O)C(CH₃)₂—. In some embodiments, A is —N(R^{C})C(O)(methylene)-, and R⁴ is alkyl (e.g., methyl). In some embodiments, A is —NHC(O)CH(CH₃)—. In some embodiments, A is —NHC(O)C(CH₃)—.

In some embodiments, for Formulas (II), (II-a), and (II-b), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (II), (II-a), and (II-b), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (II), (II-a), and (II-b), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂-)z, wherein z is an integer selected from 1 to 10. In some embodiments, z is an integer selected from 1 to 5. In some embodiments, M is —OCH₂CH₂—, (—OCH₂CH₂-)₂, (—OCH₂CH₂-)₃, (—OCH₂CH₂-)₄, or (—OCH₂CH₂-)₅. In some embodiments, M is —OCH₂CH₂—, (—OCH₂CH₂-)₂, (—OCH₂CH₂-)₃, or (—OCH₂CH₂-)₄. In some embodiments, M is (—OCH₂CH₂-)₃.

In some embodiments, for Formulas (II), (II-a), and (II-b), P is heteroaryl. In some embodiments, for Formulas (II) and (II-a), P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is triazolyl. In some embodiments, P is 1,2,3-triazolyl. In some embodiments, P is

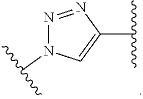 [SMILES: *c1cn(*)nn1]

In some embodiments, P is triazolyl substituted by one or more R⁴. In some embodiments, R⁴ is deuterium, alkyl, alkenyl, alkynyl, heteroalkyl, halogen, cyano, azido, —N(R^{C1})(R^{D1}), —N(R^{C1})C(O)R^{B1}, —C(O)N(R^{C1}), —S(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷. In some embodiments, R⁴ is deuterium, alkyl, heteroalkyl, halogen, cyano, or azido, wherein each alkyl and heteroalkyl is optionally substituted by one or more R⁷ (e.g., halogen). In some embodiments, P is

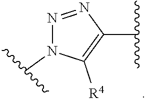

In some embodiments, P is triazolyl substituted by R⁴ (e.g., halogen). In some embodiments, R⁴ is deuterium, alkyl or halogen. In some embodiments, R⁴ is halogen (e.g., fluoro, chloro, bromo). In some embodiments, R⁴ is alkyl (e.g., —CH₃, —CH₂CH₃, —CF₃, —CH₂F, —CHF₂). In some embodiments, R⁴ is chloro. In some embodiments, P is

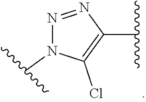 [SMILES: *c1nnn(*)c1Cl]

In some embodiments, P is

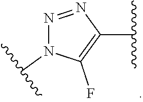 [SMILES: *c1nnn(*)c1F]

In some embodiments, P is

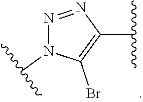 [SMILES: *c1nnn(*)c1Br]

In some embodiments, P is

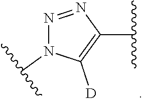 [SMILES: *c1nnn(*)c1[2H]]

In some embodiments, P is

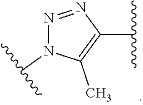 [SMILES: *c1nnn(*)c1C]

In some embodiments, P is

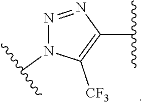 [SMILES: *c1nnn(*)c1C(F)(F)F]

In some embodiments, P is

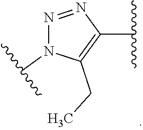 [SMILES: *c1nnn(*)c1CC]

In some embodiments, P is

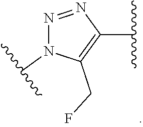 [SMILES: *c1nnn(*)c1CF]

In some embodiments, P is

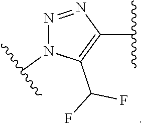 [SMILES: *c1nnn(*)c1C(F)F]

In some embodiments, for Formulas (II), (II-a), and (II-b), Z is heterocyclyl. In some embodiments, Z is monocyclic or bicyclic heterocyclyl. In some embodiments, Z is a 4-membered heterocyclyl, 5-membered heterocyclyl, or 6-membered heterocyclyl. In some embodiments, Z is a 4-membered heterocyclyl. In some embodiments, Z is a 6-membered heterocyclyl. In some embodiments, Z is a nitrogen-containing heterocyclyl. In some embodiments, Z is a sulfur-containing heterocyclyl. In some embodiments, Z is a 4-membered nitrogen-containing heterocyclyl. In some embodiments, Z is a 6-membered nitrogen-containing heterocyclyl. In some embodiments, Z is a 6-membered sulfur-containing heterocyclyl. In some embodiments, Z is a 6-membered heterocyclyl containing a nitrogen atom and a sulfur atom. In some embodiments, Z is a 4-membered nitrogen heterocyclyl optionally substituted with 1 R⁵ (e.g., —S(O)ₓR^{E1}). In some embodiments, R⁵ is —S(O)₂CH₃.

In some embodiments, Z is 3-(methylsulfonyl)azetidinyl. In some embodiments, Z is

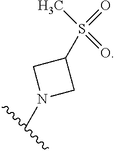 [SMILES: *N1CC(S(C)(=O)=O)C1]

In some embodiments, Z is thiomorpholinyl-1,1-dioxidyl. In some embodiments, Z is

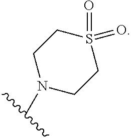 [SMILES: *N1CCS(=O)(=O)CC1]

In some embodiments, the compound of Formula (II) or (II-a) is a compound of Formula (II-c).

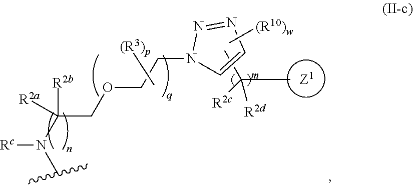

or a pharmaceutically acceptable salt thereof, wherein Ring Z¹ is heterocyclyl optionally substituted with 1-5 R⁵; R^{C} is hydrogen, alkyl, alkenyl, —C(O)(C₁-C₆-alkyl), or —C(O)(C₁-C₆-alkenyl), wherein each alkyl and alkenyl is optionally substituted with 1-6 R⁶; each of R^{2a}, R^{2b}, R^{2c}, and R^{2d} is independently hydrogen, alkyl, heteroalkyl, halo, or amino; or R^{2a} and R^{2b} or R^{2e} and R^{2d} are taken together to form an oxo group; each of R³, R⁵ and R⁶ is independently alkyl, heteroalkyl, halogen, oxo, —OR^{A1}, —C(O)OR^{A1}, —C(O)R^{B1}, —SR^{E1}, —S(O)ₓR^{E1}, or —OS(O)ₓR^{E1}; each R¹⁰ is independently alkyl, heteroalkyl, halo, cyano, nitro, or amino; each R^{A1}, R^{B1}, and R^{E1} is independently hydrogen, alkyl, or heteroalkyl; m and n are each independently 1, 2, 3, 4, 5, or 6; w is 0, 1, or 2; each of q and p is independently an integer from 0 to 25; and x is 0, 1, or 2.

In some embodiments, Ring Z¹ is heterocyclyl. In some embodiments, Ring Z¹ is nitrogen-containing heterocyclyl. In some embodiments, Ring Z¹ is 4-membered heterocyclyl or 6-membered heterocyclyl. In some embodiments, Ring Z¹ is heterocyclyl substituted with 1 R⁵. In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃).

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, w is 0. In some embodiments, w is 1. In some embodiments, R¹⁰ is halo (e.g., Cl).

In some embodiments, the compound of Formula (II-c) is a compound of Formula (II-d):

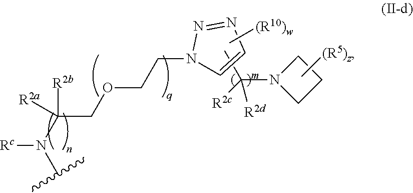

or a pharmaceutically acceptable salt thereof, wherein R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2e} and R^{2d} are taken together to form an oxo group; each of R⁵ and R⁶ is independently alkyl, heteroalkyl, halogen, oxo, —S(O)ₓR^{E1}, or —OS(O)ₓR^{E1}; each R¹⁰ is independently alkyl, heteroalkyl, halo, cyano, nitro, or amino; R^{E1} is independently hydrogen, alkyl, or heteroalkyl; m and n are each independently 1, 2, 3, 4, 5, or 6; w is 0, 1, or 2; q is an integer from 0 to 25; x is 0, 1, or 2; and z is 0, 1, 2, 3, 4, 5, or 6.

In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃). In some embodiments, z is 1.

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1. In some embodiments, w is 0.

In some embodiments, the compound of Formula (II) is a compound of Formula (II-e):

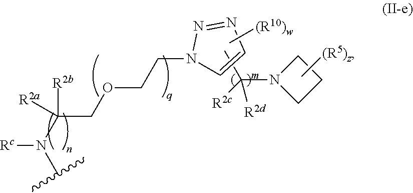

or a pharmaceutically acceptable salt thereof, wherein R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2e} and R^{2d} are taken together to form an oxo group; each of R⁵ and R⁶ is independently alkyl, heteroalkyl, halogen, oxo, —S(O)ₓR^{E1}, or —OS(O)ₓR^{E1}; each R¹⁰ is independently deuterium, alkyl, heteroalkyl, halo, cyano, azido, nitro, or amino, wherein each alkyl or heteroalkyl is optionally substituted by one or more R¹¹; each R¹¹ is independently alkyl, alkenyl, alkynyl, heteroalkyl, halogen, cyano, oxo, hydroxyl, cycloalkyl, or heterocyclyl; R^{E1} is independently hydrogen, alkyl, or heteroalkyl; m and n are each independently 1, 2, 3, 4, 5, or 6; w is 0, 1, or 2; q is an integer from 0 to 25; x is 0, 1, or 2; and z is 0, 1, 2, 3, 4, 5, or 6.

In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃). In some embodiments, z is 1.

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen).

In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1. In some embodiments, w is 0.

In some embodiments, the compound of Formula (II) is a compound of Formula (II-f):

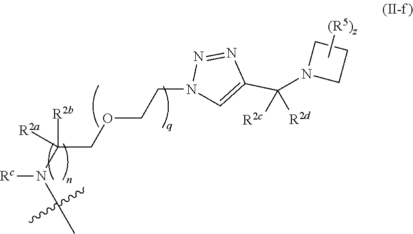

or a pharmaceutically acceptable salt thereof, wherein R^{C} is independently hydrogen, alkyl, —N(R^{C})C(O)R^{B}, —N(R^{c})C(O)(C₁-C₆-alkyl), or —N(R^{c})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R⁵ and R⁶ is independently alkyl, heteroalkyl, halogen, oxo, —S(O)ₓR^{E1}, or —OS(O)ₓR^{E1}; R^{E1} is independently hydrogen, alkyl, or heteroalkyl; n is 1, 2, 3, 4, 5, or 6; q is an integer from 0 to 25; x is 0, 1, or 2; and z is 0, 1, 2, 3, 4, 5, or 6.

In some embodiments, R⁵ is —S(O)ₓR^{E1}. In some embodiments, R^{E1} is alkyl (e.g., —CH₃).

In some embodiments, x is 2. In some embodiments, R⁵ is —S(O)₂(CH₃). In some embodiments, z is 1.

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1.

In some embodiments, the compound of Formula (II) is a compound of Formula (II-g):

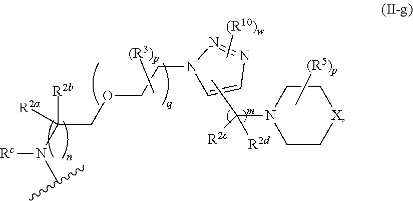

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2e} and R^{2d} are taken together to form an oxo group; R^{C} is hydrogen, alkyl, —N(R^{c})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R³, R⁵, and R⁶ is independently alkyl, heteroalkyl, halogen, oxo, —OR^{A1}, —C(O)OR^{A1}, —C(O)R^{B1}, —SR^{E1}, —S(O)ₓR^{E1}, or —OS(O)ₓR^{E1}; each R¹⁰ is independently alkyl, heteroalkyl, halo, cyano, nitro, or amino; each R^{A1}, R^{B1}, and R^{E1} is independently hydrogen, alkyl, or heteroalkyl; m and n are each independently 1, 2, 3, 4, 5, or 6; w is 1; each of q and p is independently an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R^{C} is independently, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, R¹⁰ is halo (e.g., Cl).

In some embodiments, the compound of Formula (II-g) is a compound of Formula (II-h):

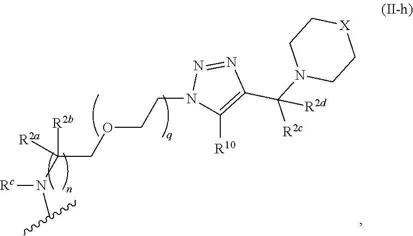

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2e} and R^{2d} are taken together to form an oxo group; R^{C} is hydrogen, alkyl, —N(R^{c})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R⁶ is independently alkyl, heteroalkyl, halogen, oxo, —OR^{A}1, —C(O)OR^{A1}, —C(O)R^{B1}; R¹⁰ is independently alkyl, heteroalkyl, halo, cyano, nitro, or amino; each R^{A1} and R^{B1} is independently hydrogen, alkyl, or heteroalkyl; n is 1, 2, 3, 4, 5, or 6; q is an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, R¹⁰ is halo (e.g., Cl).

In some embodiments, the compound of Formula (II) is a compound of Formula (II-i):

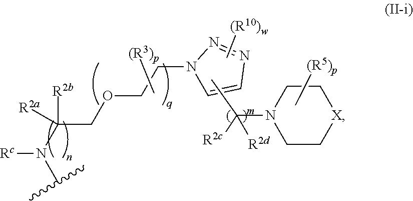

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2e} and R^{2d} are taken together to form an oxo group; R^{C} is independently hydrogen, alkyl, —N(R^{c})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R³, R⁵, and R⁶ is independently alkyl, heteroalkyl, halogen, oxo, —OR^{A1}, —C(O)OR^{A1}, —C(O)R^{B1}, —SR^{E1}, —S(O)ₓR^{E1}, or —OS(O)ₓR^{E1}; each R¹⁰ is independently deuterium, alkyl, heteroalkyl, halo, cyano, azido, nitro, or amino, wherein each alkyl or heteroalkyl is optionally substituted by one or more R¹¹; each R¹¹ is independently alkyl, alkenyl, alkynyl, heteroalkyl, halogen, cyano, oxo, hydroxyl, cycloalkyl, or heterocyclyl; each R^{A1}, R^{B1}, and R^{E1} is independently hydrogen, alkyl, or heteroalkyl; m and n are each independently 1, 2, 3, 4, 5, or 6; w is 1; each of q and p is independently an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen). In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0.

In some embodiments, the compound of Formula (II-i) is a compound of Formula (II-j):

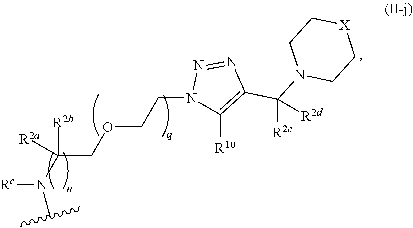

or a pharmaceutically acceptable salt thereof, wherein X is C(R′)(R″), N(R′), or S(O)ₓ; each of R′ and R″ is independently hydrogen, alkyl, or halogen; each of R^{2a}, R^{2b}, R^{2c}, and R^{2d} is independently hydrogen, alkyl, heteroalkyl, or halogen; or R^{2a} and R^{2b} or R^{2e} and R^{2d} are taken together to form an oxo group; R^{C} is independently hydrogen, alkyl, —N(R^{c})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R⁶ is independently alkyl, heteroalkyl, halogen, oxo, —OR^{A1}, —C(O)OR^{A1}, —C(O)R^{B1}; R¹⁰ is deuterium, alkyl, heteroalkyl, halo, cyano, azido, nitro, or amino, wherein each alkyl or heteroalkyl is optionally substituted by one or more R¹¹; each R¹¹ is independently alkyl, alkenyl, alkynyl, heteroalkyl, halogen, cyano, oxo, hydroxyl, cycloalkyl, or heterocyclyl; each R^{A1} and R^{B1} is independently hydrogen, alkyl, or heteroalkyl; n is 1, 2, 3, 4, 5, or 6; q is an integer from 0 to 25; and x is 0, 1, or 2.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen).

In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 3. In some embodiments, m is 1.

In some embodiments, the compound of Formula (II) is a compound of Formula (II-k):

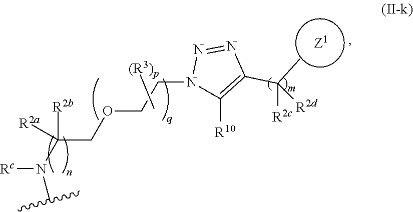

or a pharmaceutically acceptable salt thereof, wherein Ring Z¹ is heterocyclyl optionally substituted with 1-5 R⁵; R^{C} is independently hydrogen, alkyl, alkenyl, —C(O)(C₁-C₆-alkyl), or —C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen, alkyl, heteroalkyl, halo, or amino; or R^{2a} and R^{2b} or R^{2e} and R^{2d} are taken together to form an oxo group; each of R³, R⁵ and R⁶ is independently alkyl, heteroalkyl, halogen, oxo, —OR^{A1}, —C(O)OR^{A1}, —C(O)R^{B1}, —SR^{E1}, —S(O)ₓR^{E1}, or —OS(O)ₓR^{E1}; R¹⁰ is hydrogen, deuterium, alkyl, heteroalkyl, halo, cyano, azido, nitro, or amino, wherein each alkyl or heteroalkyl is optionally substituted by one or more R¹¹; each R¹¹ is independently alkyl, alkenyl, alkynyl, heteroalkyl, halogen, cyano, oxo, hydroxyl, cycloalkyl, or heterocyclyl; each R^{A1}, R^{B1}, and R^{E1} is independently hydrogen, alkyl, or heteroalkyl; m and n are each independently 1, 2, 3, 4, 5, or 6; w is 0, 1, or 2; each of q and p is independently an integer from 0 to 25; and x is 0, 1, or 2.

In some embodiments, Ring Z¹ is heterocyclyl. In some embodiments, Ring Z¹ is nitrogen-containing heterocyclyl. In some embodiments, Ring Z¹ is 4-membered heterocyclyl or 6-membered heterocyclyl. In some embodiments, Ring Z¹ is heterocyclyl substituted with 1 R⁵. In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃).

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen). In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, w is 0. In some embodiments, w is 1. In some embodiments, R¹⁰ is halo (e.g., Cl).

In some embodiments, the compound of Formula (II) is a compound of Formula (II-1):

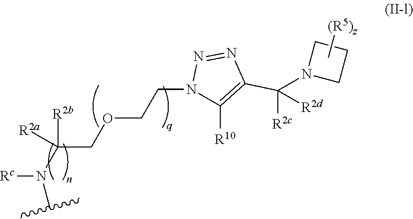

or a pharmaceutically acceptable salt thereof, wherein R^{C} is independently hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R⁵ and R⁶ is independently alkyl, heteroalkyl, halogen, oxo, —S(O)ₓR^{E1}, or —OS(O)ₓR^{E1}; R¹⁰ is deuterium, alkyl, heteroalkyl, halo, cyano, azido, nitro, or amino, wherein each alkyl or heteroalkyl is optionally substituted by one or more R¹¹; each R¹¹ is independently alkyl, alkenyl, alkynyl, heteroalkyl, halogen, cyano, oxo, hydroxyl, cycloalkyl, or heterocyclyl; R^{E1} is independently hydrogen, alkyl, or heteroalkyl; n is 1, 2, 3, 4, 5, or 6; q is an integer from 0 to 25; x is 0, 1, or 2; and z is 0, 1, 2, 3, 4, 5, or 6.

In some embodiments, R⁵ is —S(O)ₓR^{E1}. In some embodiments, R^{E1} is alkyl (e.g., —CH₃). In some embodiments, x is 2. In some embodiments, R⁵ is —S(O)₂(CH₃). In some embodiments, z is 1.

In some embodiments, each of R^{2a}, R^{2b}, R^{2c}, and R^{2d} is independently hydrogen.

In some embodiments, R¹⁰ is deuterium, alkyl, heteroalkyl, halogen, cyano, or azido, wherein each alkyl and heteroalkyl is optionally substituted by one or more R¹¹ (e.g., halogen).

In some embodiments, R¹⁰ is deuterium, alkyl, or halogen. In some embodiments, R¹⁰ is halogen (e.g., fluoro, chloro, bromo). In some embodiments, R¹⁰ is alkyl (e.g., —CH₃, —CH₂CH₃, —CF₃, —CH₂F, —CHF₂).

In some embodiments, R^{C} is hydrogen, —C(O)(C₁-C₆-alkyl), or —C(O)(C₁-C₆-alkenyl). In some embodiments, R^{C} is hydrogen.

In some embodiments, n is 1. In some embodiments, q is 2, 3, 4, or 5. In some embodiments, q is 3. In some embodiments, m is 1.

In some embodiments, the compound of Formula (II) comprises a compound shown in Table 2, or a pharmaceutically acceptable salt thereof.

TABLE 2
Exemplary compounds of Formula (II)

| Compound No. | Structure |
|---|---|
| 400 | 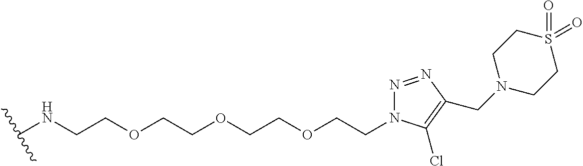 [SMILES: *NCCOCCOCCOCCn1nnc(CN2CCS(=O)(=O)CC2)c1Cl] |
| 401 | 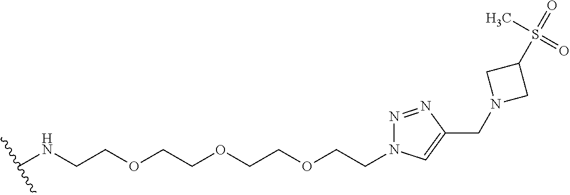 [SMILES: *NCCOCCOCCOCCn1cc(CN2CC(S(C)(=O)=O)C2)nn1] |
| 402 | 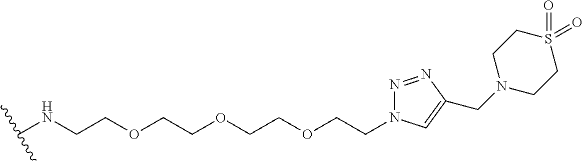 [SMILES: *NCCOCCOCCOCCn1cc(CN2CCS(=O)(=O)CC2)nn1] |
| 403 | 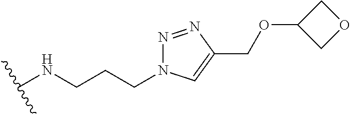 [SMILES: *NCCCn1cc(COC2COC2)nn1] |
| 404 | 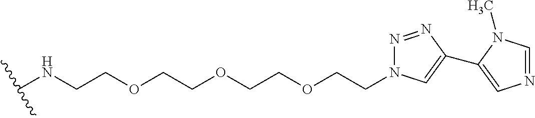 [SMILES: *NCCOCCOCCOCCn1cc(-c2cncn2C)nn1] |
| 405 | 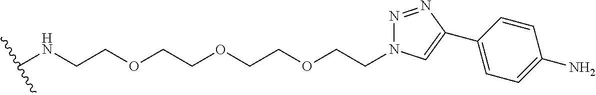 [SMILES: *NCCOCCOCCOCCn1cc(-c2ccc(N)cc2)nn1] |
| 406 | 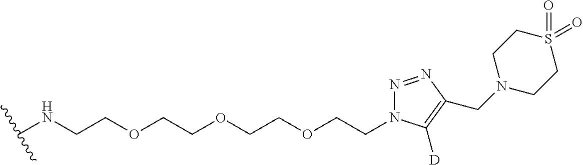 [SMILES: *NCCOCCOCCOCCn1nnc(CN2CCS(=O)(=O)CC2)c1[2H]] |
| 407 | 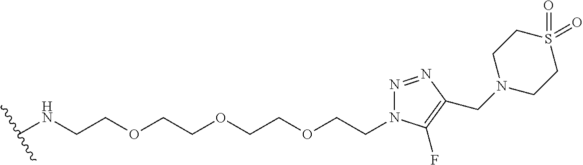 [SMILES: *NCCOCCOCCOCCn1nnc(CN2CCS(=O)(=O)CC2)c1F] |
| 408 | 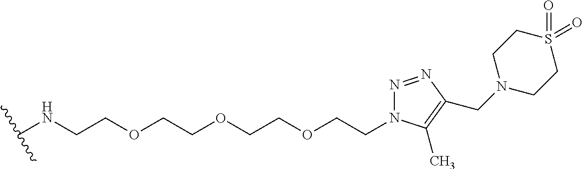 [SMILES: *NCCOCCOCCOCCn1nnc(CN2CCS(=O)(=O)CC2)c1C] |
| 409 | 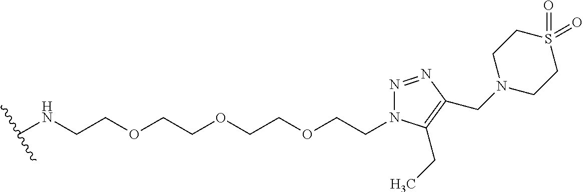 [SMILES: *NCCOCCOCCOCCn1nnc(CN2CCS(=O)(=O)CC2)c1CC] |
| 410 | 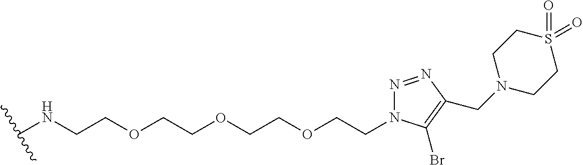 [SMILES: *NCCOCCOCCOCCn1nnc(CN2CCS(=O)(=O)CC2)c1Br] |
| 411 | 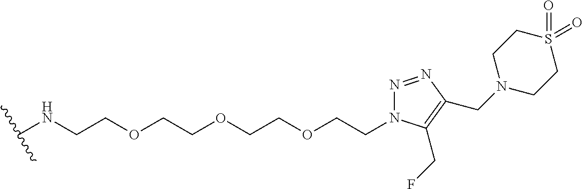 [SMILES: *NCCOCCOCCOCCn1nnc(CN2CCS(=O)(=O)CC2)c1CF] |
| 412 | 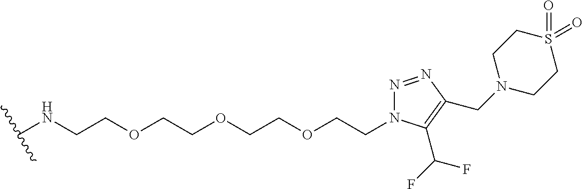 [SMILES: *NCCOCCOCCOCCn1nnc(CN2CCS(=O)(=O)CC2)c1C(F)F] |
| 413 | 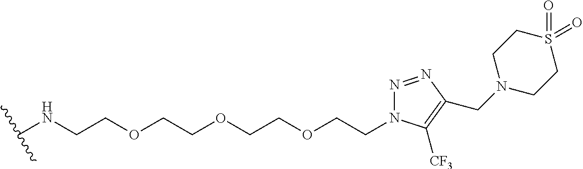 [SMILES: *NCCOCCOCCOCCn1nnc(CN2CCS(=O)(=O)CC2)c1C(F)(F)F] |
| 414 | 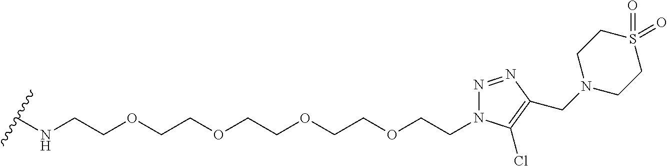 [SMILES: *NCCOCCOCCOCCOCCn1nnc(CN2CCS(=O)(=O)CC2)c1Cl] |
| 415 | 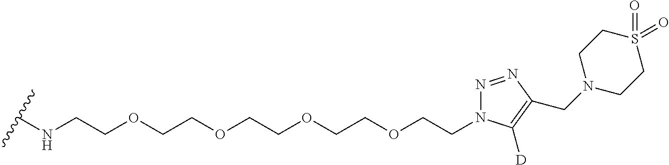 [SMILES: *NCCOCCOCCOCCOCCn1nnc(CN2CCS(=O)(=O)CC2)c1[2H]] |
| 416 | 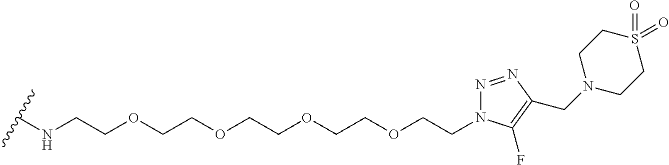 [SMILES: *NCCOCCOCCOCCOCCn1nnc(CN2CCS(=O)(=O)CC2)c1F] |
| 417 | 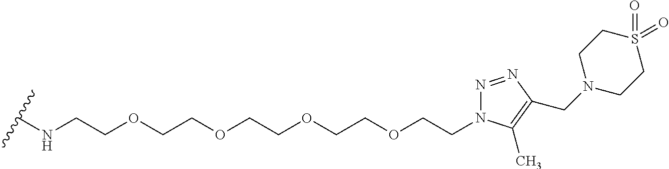 [SMILES: *NCCOCCOCCOCCOCCn1nnc(CN2CCS(=O)(=O)CC2)c1C] |
| 418 | 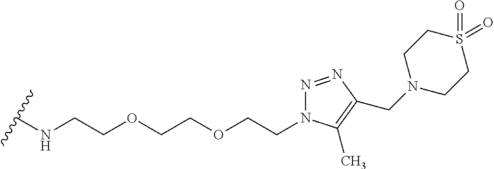 [SMILES: *NCCOCCOCCn1nnc(CN2CCS(=O)(=O)CC2)c1C] |
| 419 | 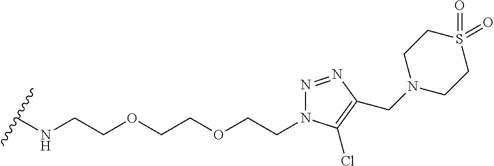 [SMILES: *NCCOCCOCCn1nnc(CN2CCS(=O)(=O)CC2)c1Cl] |
| 420 | 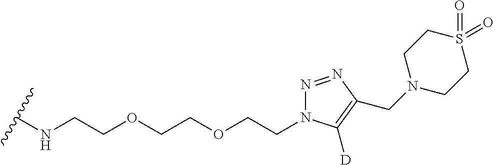 [SMILES: *NCCOCCOCCn1nnc(CN2CCS(=O)(=O)CC2)c1[2H]] |
| 421 | 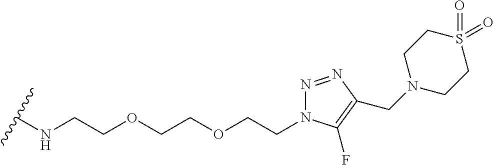 [SMILES: *NCCOCCOCCn1nnc(CN2CCS(=O)(=O)CC2)c1F] |
| 422 | 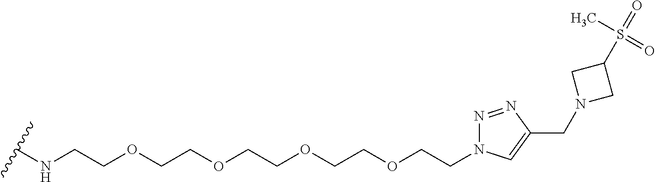 [SMILES: *NCCOCCOCCOCCOCCn1cc(CN2CC(S(C)(=O)=O)C2)nn1] |
| 423 | 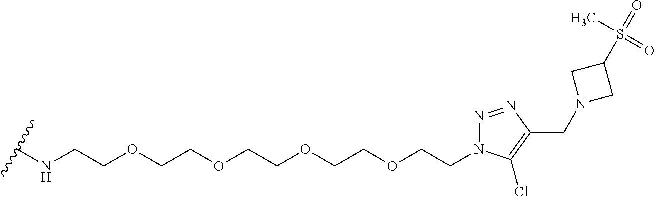 [SMILES: *NCCOCCOCCOCCOCCn1nnc(CN2CC(S(C)(=O)=O)C2)c1Cl] |
| 424 | 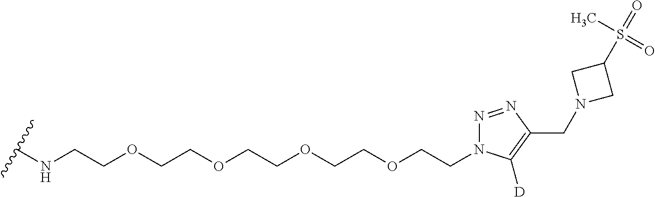 [SMILES: *NCCOCCOCCOCCOCCn1nnc(CN2CC(S(C)(=O)=O)C2)c1[2H]] |
| 425 | 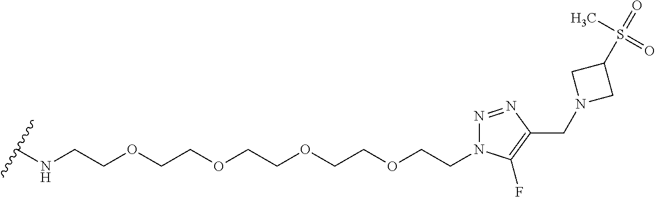 [SMILES: *NCCOCCOCCOCCOCCn1nnc(CN2CC(S(C)(=O)=O)C2)c1F] |
| 426 | 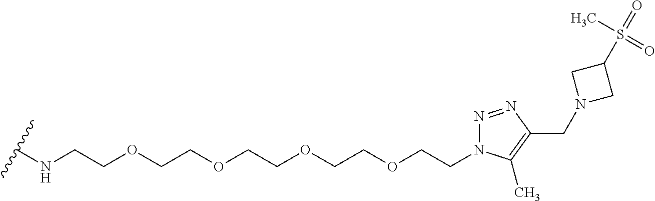 [SMILES: *NCCOCCOCCOCCOCCn1nnc(CN2CC(S(C)(=O)=O)C2)c1C] |
| 427 | 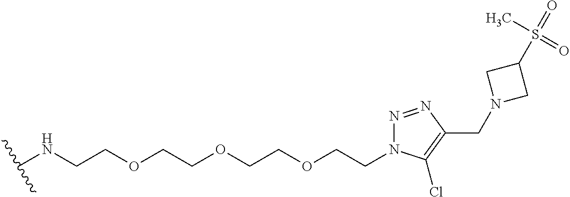 [SMILES: *NCCOCCOCCOCCn1nnc(CN2CC(S(C)(=O)=O)C2)c1Cl] |
| 428 | 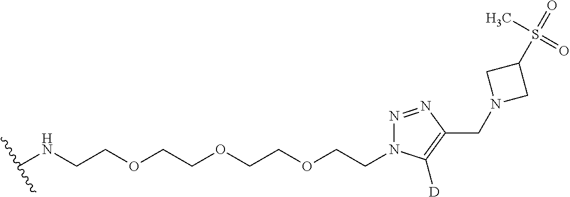 [SMILES: *NCCOCCOCCOCCn1nnc(CN2CC(S(C)(=O)=O)C2)c1[2H]] |
| 429 | 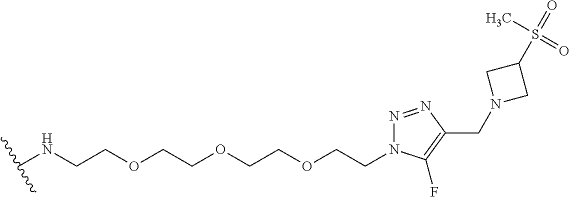 [SMILES: *NCCOCCOCCOCCn1nnc(CN2CC(S(C)(=O)=O)C2)c1F] |
| 430 | 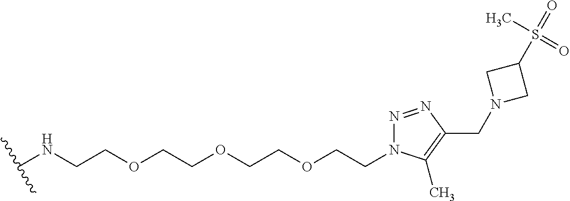 [SMILES: *NCCOCCOCCOCCn1nnc(CN2CC(S(C)(=O)=O)C2)c1C] |
| 431 | 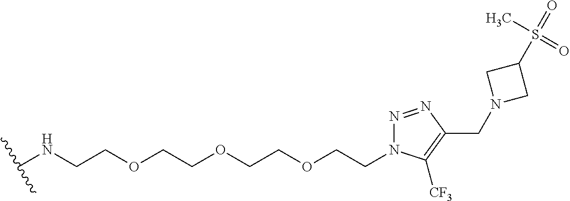 [SMILES: *NCCOCCOCCOCCn1nnc(CN2CC(S(C)(=O)=O)C2)c1C(F)(F)F] |
| 432 | 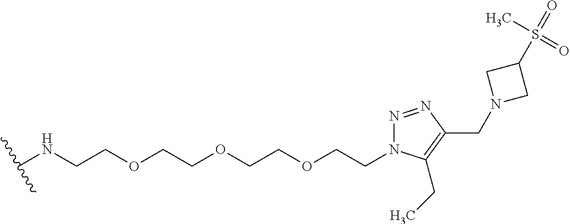 [SMILES: *NCCOCCOCCOCCn1nnc(CN2CC(S(C)(=O)=O)C2)c1CC] |

In some embodiments, the compound is a compound of Formula (II), (II-a), (II-b), (II-c), (II-d), or a pharmaceutically acceptable salt thereof. In an embodiment, the compound is Compound 400.

In some embodiments, the compound is a compound of Formula (II), (II-a), (II-b), (II-e), (II-f), or a pharmaceutically acceptable salt thereof. In an embodiment, the compound is Compound 401.

In some embodiments, the compound is not Compound 402, Compound 403, Compound 404, or Compound 405.

Modified Polymers

A polymer modified with a compound of Formula (II) or a pharmaceutically acceptable salt thereof may be a linear, branched, or cross-linked polymer, or a polymer of selected molecular weight ranges, degree of polymerization, viscosity or melt flow rate. Branched polymers can include one or more of the following types: star polymers, comb polymers, brush polymers, dendronized polymers, graft-co(polymers), ladders, and dendrimers. A polymer may be a thermoresponsive polymer, e.g., a gel (e.g., becomes a solid or liquid upon exposure to heat or a certain temperature) or a photocrosslinkable polymer.

Exemplary polymers include polystyrene, polyethylene, polypropylene, polyacetylene, poly(vinyl chloride) (PVC), polyolefin copolymers, poly(urethane)s, polyacrylates and polymethacrylates, polyacrylamides and polymethacrylamides, poly(methyl methacrylate), poly(2-hydroxyethyl methacrylate), polyesters, polysiloxanes, polydimethylsiloxane (PDMS), polyethers, poly(orthoester), poly(carbonates), poly(hydroxyalkanoate)s, polyfluorocarbons, polyether ether ketone (PEEK), polytetrafluoroethylene (PTFE), silicones, epoxy resins, poly-paraphenylene terephthalanide, polyethylene, terephthalate. (PET), polyethylene glycol (PEG), nylon, polyalkenes, phenolic resins, natural and synthetic elastomers, adhesives and sealants, polyolefins, polysulfones, polyacrylonitrile, biopolymers such as polysaccharides and natural latex, collagen, cellulosic polymers (e.g., alkyl celluloses, etc.), polyethylene glycol and 2-hydroxyethyl methacrylate (HEMA), polysaccharides, poly(glycolic acid), poly(L-lactic acid) (PLLA), poly(lactic glycolic acid) (PLGA), a polydioxanone (PDA), or racemic poly(lactic acid), polycarbonates, (e.g., polyamides (e.g., nylon)), poly(l-isopropylacrylamide-co-acrylic acid)-poly(L-lactic acid) (NAL); poly(N-isopropyl acrylamide) (PNIPAM) grafted to other polymers such as carboxymethylcellulose (CMC), copolymers or polymers including block copolymers and end-functionalized polymers, composites or copolymers containing thermo-sensitive poly(2-ethoxyethyl vinyl ether) and/or poly(hydroxyethyl vinyl ether), fluoroplastics, carbon fiber, agarose, alginate, chitosan, and blends or copolymers thereof. In some embodiments, the polymer comprises poly(ethylene oxide). In some embodiments, the polymer comprises polyvinyl alcohol (PVA). In some embodiments, a polymer is made up of a single type of repeating monomeric unit. In other embodiments, a polymer is made up of different types of repeating monomeric units (e.g., two types of repeating monomeric units, three types of repeating monomeric units, e.g., a polymeric blend).

In some embodiments, the polymer may comprise a polyurethane, polyurea, polyether(amide), PEBA, thermoplastic elastomeric olefin, copolyester, and styrenic thermoplastic elastomer, PEO-PPO-PEO (poloxamers) or combinations thereof.

In an embodiment, the polymer is a cellulose, e.g., carboxymethyl cellulose. In an embodiment, the polymer is a polylactide, a polyglycoside or a polycaprolactone. In an embodiment, the polymer is a hyaluronate, e.g., sodium hyaluronate. In an embodiment, the polymer is a polyurethane, a PVP, or a PIE In an embodiment, the polymer is a collagen, elastin or gelatin.

In some embodiments, the polymer is a polyethylene. Exemplary polyethylenes include ultra-low-density polyethylene (ULDPE) (e.g., with polymers with densities ranging from 0.890 to 0.905 g/cm³, containing comonomer); very-low-density polyethylene (VLDPE) (e.g., with polymers with densities ranging from 0.905 to 0.915 g/cm³, containing comonomer); linear low-density polyethylene (LLDPE) (e.g., with polymers with densities ranging from 0.915 to 0.935 g/cm³, contains comonomer); low-density polyethylene (LDPE) (e.g., with polymers with densities ranging from about 0.915 to 0.935 g/m³); medium density polyethylene (MDPE) (e.g., with polymers with densities ranging from 0.926 to 0.940 g/cm³, may or may not contain comonomer); high-density polyethylene (HDPE) (e.g., with polymers with densities ranging from 0.940 to 0.970 g/cm³, may or may not contain comonomer).

In some embodiments, the polymer is a polypropylene. Exemplary polypropylenes include homopolymers, random copolymers (homophasic copolymers), and impact copolymers (heterophasic copolymers), e.g., as described in McKeen, Handbook of Polymer Applications in Medicine and Medical Devices, 3—Plastics Used in Medical Devices, (2014):21-53.

In some embodiments, the polymer is a polystyrene. Exemplary polystyrenes include general purpose or crystal (PS or GPPS), high impact (HIPS), and syndiotactic (SPS) polystyrene.

In some embodiments, the polymer is a thermoplastic elastomer (TPE). Exemplary TPEs include (i) TPA-polyamide TPE, comprising a block copolymer of alternating hard and soft segments with amide chemical linkages in the hard blocks and ether and/or ester linkages in the soft blocks; (ii) TPC-copolyester TPE, consisting of a block copolymer of alternating hard segments and soft segments, the chemical linkages in the main chain being ester and/or ether; (iii) TPO-olefinic TPE, consisting of a blend of a polyolefin and a conventional rubber, the rubber phase in the blend having little or no cross-linking; (iv) TPS-styrenic TPE, consisting of at least a triblock copolymer of styrene and a specific diene, where the two end blocks (hard blocks) are polystyrene and the internal block (soft block or blocks) is a polydiene or hydrogenated polydiene; (v) TPU-urethane TPE, consisting of a block copolymer of alternating hard and soft segments with urethane chemical linkages in the hard blocks and ether, ester or carbonate linkages or mixtures of them in the soft blocks; (vi) TPV-thermoplastic rubber vulcanizate consisting of a blend of a thermoplastic material and a conventional rubber in which the rubber has been cross-linked by the process of dynamic vulcanization during the blending and mixing step; and (vii) TPZ-unclassified TPE comprising any composition or structure other than those grouped in TPA, TPC, TPO, TPS, TPU, and TPV.

In some embodiments, the polymer is a hydrogel-forming polymer. Hydrogel-forming polymers comprise a hydrophilic structure that renders them capable of holding large amounts of water in a three-dimensional network. Hydrogel-forming polymers may include polymers which form homopolymeric hydrogels, copolymeric hydrogels, or multipolymer interpenetrating polymeric hydrogels, and may be amorphous, semicrystalline, or crystalline in nature, e.g., as described in Ahmed (2015) J Adv Res 6:105-121. Exemplary hydrogel-forming polymers include proteins (e.g., collagen), gelatin, polysaccharides (e.g., starch, alginate, hyaluronate, agarose), and synthetic polymers. In some embodiments, the hydrogel-forming polymer is a polysaccharide.

In some embodiments, the polymer is a polysaccharide. Exemplary polysaccharides include alginate, agar, agarose, carrageenan, hyaluronate, amylopectin, glycogen, gelatin, cellulose, amylose, chitin, chitosan, or a derivative or variant thereof, e.g., as described in Laurienzo (2010), Mar Drugs 9:2435-65. A polymer may comprise heparin, chondoitin sulfate, dermatan, dextran, or carboxymethylcellulose. In some embodiments, a polysaccharide is a cross-linked polymer. In some embodiments, a polysaccharide is a cell-surface polysaccharide.

In some embodiments, the polymer is an alginate. Algnate is a polysaccharide made up of β-D-mannuronic acid (M) and α-L-guluronic acid (G). In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1. In some embodiments, the alginate has an approximate molecular weight of <75 kDa, and optionally a G:M ratio of >1.5. In some embodiments, the alginate has an approximate molecular weight of 75 kDa to 150 kDa and optionally a G:M ratio of ≥1.5. In some embodiments, the alginate has an approximate molecular weight of 150 to 250 kDa and optionally a G:M ratio of 1.5.

A polymer (e.g., any of the polymers described herein, for example, any of the alginates described herein) modified with a compound of Formula (II) or a pharmaceutically acceptable salt thereof may be modified on one or more monomeric units. In some embodiments, at least 0.5 percent of the monomers of a polymer are modified with a compound of Formula (II) (e.g., at least 1, 2.5, 5, 10, 15, 20, 25, 30, 35, 40, 45, 50, 55, 60, 65, 70, 75, 80, 85, 90, 95, 99 percent, or more of the monomers of a polymer are modified with a compound of Formula (II). In some embodiments, 0.5 to 50%, 10 to 90%, 10 to 50%, or 25-75%, of the monomers of a polymer are modified with a compound of Formula (II). In some embodiments, 1 to 20% of the monomers of a polymer are modified with a compound of Formula (II). In some embodiments, 1 to 10% of the monomers of a polymer are modified with a compound of Formula (II).

In some embodiments, the polymer (when modified with a compound of Formula II) comprises an increase in % N (as compared with unmodified polymer) of at least 0.1, 0.2, 0.5, 1.0, 1.5, 2, 2.5, 3, 3.5, 4, 4.5, 5, 5.5, 6, 6.5, 7, 7.5, 8, 8.5, 9, 9.5, or 10% N by weight, where % N is determined by elemental analysis and corresponds to the amount of compound of Formula II in the modified polymer.

In some embodiments, the polymer (when modified with a compound of Formula II) comprises an increase in % N (as compared with unmodified polymer) of 0.1 to 10% N by weight, where % N is determined by elemental analysis and corresponds to the amount of compound of Formula II in the modified polymer.

In some embodiments, the polymer (when modified with a compound of Formula II) comprises an increase in % N (as compared with unmodified polymer) of 0.1 to 2% N by weight, where % N is determined by elemental analysis and corresponds to the amount of compound of Formula II in the modified polymer.

In some embodiments, the polymer (when modified with a compound of Formula II) comprises an increase in % N (as compared with unmodified polymer) of 2 to 4% N by weight, where % N is determined by elemental analysis and corresponds to the amount of compound of Formula II in the modified polymer.

In some embodiments, the polymer (when modified with a compound of Formula II) comprises an increase in % N (as compared with unmodified polymer) of 4 to 8% N by weight, where % N is determined by elemental analysis and corresponds to the amount of compound of Formula II in the modified polymer.

In some embodiments, any of the polymers described herein (e.g., an alginate) is modified with a Formula (II-a), (II-b), (II-c), (II-d), (II-e), (II-f), (II-g), (II-h), (II-i), (II-j), (II-k), (II-1)) or a pharmaceutically acceptable salt thereof. In some embodiments, the polymer is modified with a compound of Formula (II-a). In some embodiments, the polymer is modified with a compound of Formula (II-b). In some embodiments, the polymer is modified with a compound of Formula (II-c). In some embodiments, the polymer is modified with a compound of Formula (II-d). In some embodiments, the polymer is modified with a compound of Formula (II-e). In some embodiments, the polymer is modified with a compound of Formula (II-f). In some embodiments, the polymer is modified with a compound of Formula (II-g). In some embodiments, the polymer is modified with a compound of Formula (II-h). In some embodiments, the polymer is modified with a compound of Formula (II-i). In some embodiments, the polymer is modified with a compound of Formula (II-j). In some embodiments, the polymer is modified with a compound of Formula (II-k). In some embodiments, the polymer is modified with a compound of Formula (II-1).

In some embodiments, the polymer (e.g., an alginate) is modified with a compound shown in Table 2. In some embodiments, the polymer is modified with Compound 400. In some embodiments, the polymer is modified with Compound 401. In some embodiments, the polymer is not modified with Compound 402, Compound 403, Compound 404, or Compound 405.

In some embodiments, a polymer (e.g., an alginate) modified with a compound of Formula (II) is not a modified polymer described in any one of WO2012/112982, WO2012/167223, WO2014/153126, WO2016/187225, WO2016/019391, WO2017/075630, WO 2017/075631, WO 2018/067615, WO 2019/169333, and US 2016-0030359.

Implantable Elements

The disclosure also features an implantable element (e.g., a device or material) comprising a compound of Formula (II) or a pharmaceutically acceptable salt thereof, as described herein. The compound of Formula (II) may be covalently or noncovalently bound to the implantable element (e.g., to a surface of the implantable element). The surface of the implantable element may comprise a material modified with a compound of Formula (II), e.g., any of the modified polymers described above. In an embodiment, the compound of Formula (II) is covalently attached to a surface (e.g., an exterior surface) of the implantable element. The implantable element comprising a compound of Formula (II) may have an improved property compared to a reference implantable element, e.g., an otherwise identical implantable element that lacks a compound of Formula (II). In an embodiment, the improved property is a reduced foreign body response to the implantable element when administered to a subject (e.g., lower amount and/or later occurrence of PFO).

In some embodiments, the implantable element comprises a cell. In some embodiments, the cell is an engineered cell. In some embodiments, the cell is entirely or partially disposed with the implantable element. The implantable element may comprise an enclosing element that encapsulates or coats a cell, in part or in whole. In an embodiment, an implantable element comprises an enclosing component that is formed, or could be formed, in situ on or surrounding a cell, e.g., a plurality of cells, e.g., a cluster of cells, or on a microcarrier, e.g., a bead, or a matrix comprising a cell or cells.

Implantable elements can include any material, such as a polymer or other material described herein. In some embodiments, an implantable element is made up of one material or many types of materials. Implantable elements can comprise non-organic or metal components or materials, e.g., steel (e.g., stainless steel), titanium, other metal or alloy. Implantable elements can include nonmetal components or materials, e.g., ceramic, or hydroxyapatite elements.

Implantable elements can include components or materials that are made of a conductive material (e.g., gold, platinum, palladium, titanium, copper, aluminum, silver, metals, any combinations of these, etc.).

Implantable elements can include more than one component, e.g., more than one component disclosed herein, e.g., more than one of a metal, plastic, ceramic, composite, or hybrid material.

Exemplary implantable elements comprise materials such as metals, metallic alloys, ceramics, polymers, fibers, inert materials, and combinations thereof. An implantable element may be completely made up of one type of material, or may just refer to a surface or the surface of an implantable element (e.g., the outer surface or an inner surface).

In some embodiments, the implantable element (e.g., a device or material) comprises a metal or a metallic alloy. Exemplary metallic or metallic alloys include comprising titanium and titanium group alloys (e.g., nitinol, nickel titanium alloys, thermo-memory alloy materials), platinum, platinum group alloys, stainless steel, tantalum, palladium, zirconium, niobium, molybdenum, nickel-chrome, chromium molybdenum alloys, or certain cobalt alloys (e.g., cobalt-chromium and cobalt-chromium-nickel alloys. For example, a metallic material may be stainless steel grade 316 (SS 316L) (comprised of Fe, <0.3% C, 16-18.5% Cr, 10-14% Ni, 2-3% Mo, <2% Mn, <1% Si, <0.45% P, and <0.03% S). In metal-containing implantable elements, the amount of metal (e.g., by % weight, actual weight) can be at least 5 percent, e.g., at least 5, 10, 20, 30, 40, 50, 60, 70, 80, 90, 95, 99 percent, or more, e.g., w/w; less than 20 percent, e.g., less than 20, 15, 10, 5, 1, 0.5, 0.1 percent, or less.

In some embodiments, the implantable element (e.g., a device or material) is a ceramic. Exemplary ceramic materials include oxides, carbides, or nitrides of the transition elements, such as titanium oxides, hafnium oxides, iridium oxides, chromium oxides, aluminum oxides, and zirconium oxides. Silicon based materials, such as silica, may also be used. In ceramic-containing implantable elements, the amount of ceramic (e.g., by % weight, actual weight) can be at least 5 percent, e.g., at least 5, 10, 20, 30, 40, 50, 60, 70, 80, 90, 95, 99 percent, or more, e.g., w/w; less than 20 percent, e.g., less than 20, 15, 10, 5, 1, 0.5, 0.1 percent, or less.

In some embodiments, an implantable element comprises a polymer (e.g., hydrogel, plastic) component. Exemplary polymers include polyethylene, polypropylene, polystyrene, polyester (e.g., PLA, PLG, or PGA, polyhydroxyalkanoates (PHAs), or other biosorbable plastic), polycarbonate, polyvinyl chloride (PVC), polyethersulfone (PES), polyacrylate (e.g., acrylic or PMMA), hydrogel (e.g., acrylic polymer or blend of acrylic and silicone polymers), polysulfone, polyetheretherketone, thermoplastic elastomers (TPE or TPU), thermoset elastomer (e.g., silicone (e.g., silicone elastomer)), poly-p-xylylene (Parylene), fluoropolymers (e.g., PTFE), and polyacrylics such as poly(acrylic acid) and/or poly(acrylamide), or mixtures thereof. In polymer-containing implantable elements, the amount of polymer (e.g., by % weight, actual weight) can be at least 5 percent, e.g., at least 5, 10, 20, 30, 40, 50, 60, 70, 80, 90, 95, 99 percent or more, e.g., w/w; less than 20 percent, e.g., less than 20, 15, 10, 5, 1, 0.5, 0.1 percent, or less.

In some embodiments, the implantable element (e.g., a device or material) comprises a polymer that is (i) modified with a compound of Formula (II) and (ii) is covalently or non-covalently associated with a component of the implantable element (e.g., the surface of the implantable element). In some embodiments, the polymer is covalently associated with a component of the implantable element (e.g., on the inner surface or outer surface of an implantable element). In some embodiments, the polymer is non-covalently associated with a component of the implantable element (e.g., on the inner surface or outer surface of an implantable element). The polymer can be applied to an implantable element by a variety of techniques in the art including, but not limited to, spraying, wetting, immersing, dipping, such as dip coating (e.g., intraoperative dip coating), painting, or otherwise applying a hydrophobic polymer to a surface of the implantable element.

In an embodiment, the implantable element comprises a flexible polymer, e.g., alginate (e.g., any of the chemically modified alginates described herein), PLA, PLG, PEG, CMC, or mixtures thereof (referred to herein as a “polymer encapsulated implantable device”).

In some embodiments, the implantable element comprises a hydrogel-forming polymer. Hydrogel-forming polymers comprise a hydrophilic structure that renders them capable of holding large amounts of water in a three-dimensional network. Hydrogel-forming polymers may include polymers which form homopolymeric hydrogels, copolymeric hydrogels, or multipolymer interpenetrating polymeric hydrogels, and may be amorphous, semicrystalline, or crystalline in nature, e.g., as described in Ahmed (2015) J Adv Res 6:105-121. Exemplary hydrogel-forming polymers include proteins (e.g., collagen), gelatin, polysaccharides (e.g., starch, alginate, hyaluronate, agarose), and synthetic polymers. In some embodiments, the hydrogel-forming polymer is a polysaccharide (e.g., alginate).

In some embodiments, the implantable element comprises a polysaccharide. Exemplary polysaccharides include alginate, agar, agarose, carrageenan, hyaluronate, amylopectin, glycogen, gelatin, cellulose, amylose, chitin, chitosan, or a derivative or variant thereof, e.g., as described in Laurienzo (2010), Mar Drugs 9:2435-65. An implantable element may comprise a polysaccharide comprising heparin, chondoitin sulfate, dermatan, dextran, or carboxymethylcellulose. In some embodiments, a polysaccharide is a cross-linked polymer. In some embodiments, a polysaccharide is a cell-surface polysaccharide.

In some embodiments, the implantable element comprises an alginate. In some embodiments, the ratio of M:G in the alginate is about 1. In some embodiments, the ratio of M:G in the alginate is less than 1. In some embodiments, the ratio of M:G in the alginate is greater than 1. In some embodiments, the alginate is any of the modified alginates described herein.

In an embodiment, an implantable element comprises is formed, or could be formed, in situ on or surrounding cell, e.g., a plurality of cells, e.g., a cluster of cells, or on a microcarrier, e.g., a bead, or a matrix comprising cell or cells.

In an embodiment, an implantable element comprises is preformed prior to combination with the enclosed cell, e.g., a plurality of cells, e.g., a cluster of cells, or on a microcarrier, e.g., a bead, or a matrix comprising cell or cells. An implantable element can include a protein or polypeptide, e.g., an antibody, protein, enzyme, or growth factor. An implantable element can include an active or inactive fragment of a protein or polypeptide, such as a glucose oxidase (e.g., for glucose sensor), kinase, phosphatase, oxygenase, hydrogenase, or reductase.

Implantable elements included herein include implantable elements that are configured with a lumen, e.g., a lumen having one, two or more openings, e.g., tubular devices, e.g., a catheter. A typical stent is an example of a device configured with a lumen and having two openings. Other examples include shunts.

Implantable elements included herein include flexible implantable elements, e.g., that are configured to conform to the shape of the body.

Implantable elements included herein include components that stabilize the location of the implantable element, e.g., an adhesive, or fastener, e.g., a torque-based or friction-based fastener, e.g., a screw or a pin.

Implantable elements included herein may be configured to monitor a substance, e.g., an exogenous substance, e.g., a therapeutic agent or toxin, or an endogenous body product, e.g., a polypeptide e.g., insulin or glucose. In some embodiments, the implantable element is a diagnostic.

Implantable elements included herein may be configured to release a substance, e.g., an exogenous substance, e.g., a therapeutic agent described herein. In some embodiments, the therapeutic agent is a compound of Formula (II) or a pharmaceutically acceptable salt thereof. In some embodiments, the therapeutic agent is a biological material. In some embodiments, the therapeutic agent is a nucleic acid (e.g., an RNA or DNA), protein (e.g., a hormone, enzyme, antibody, antibody fragment, antigen, or epitope), small molecule, lipid, drug, vaccine, or any derivative thereof.

Implantable elements herein may be configured to change conformation in response to a signal or movement of the body, e.g., an artificial joint, e.g., a knee, hip, or other artificial joint.

Exemplary implantable elements include a stent, shunt, dressing, ocular device, port, sensor, orthopedic fixation device, implant (e.g., a dental implant, ocular implant, silicon implant, corneal implant, dermal implant, intragastric implant, facial implant, hip implant, bone implant, cochlear implant, penile implant, implants for control of incontinence), skin covering device, dialysis media, drug-delivery device, artificial or engineered organ (e.g., a spleen, kidney, liver, or heart), drainage device (e.g., a bladder drainage device), cell selection system, adhesive (e.g., a cement, clamp, clip), contraceptive device, intrauterine device, defibrillator, dosimeter, electrode, pump (e.g., infusion pump) filter, embolization device, fastener, fillers, fixative, graft, hearing aid, cardio or heart-related device (e.g., pacemaker, heart valve), battery or power source, hemostatic agent, incontinence device, intervertebral body fusion device, intraoral device, lens, mesh, needle, nervous system stimulator, patch, peritoneal access device, plate, plug, pressure monitoring device, ring, transponder, and valve. Also included are devices used in one or more of anesthesiology, cardiology, clinical chemistry, otolaryngology, dentistry, gastroenterology, urology, hematology, immunology, microbiology, neurology, obstetrics/gynecology, ophthalmology, orthopedic, pathology, physical medicine, radiology, general or plastic surgery, veterinary medicine, psychiatry, surgery, and/or clinical toxicology.

Implantable elements included herein include FDA class 1, 2, or 3 devices, e.g., devices that are unclassified or not classified, or classified as a humanitarian use device (HUD).

In some embodiments, an implantable element includes encapsulated or entrapped cells or tissues. The cells or tissue can be encapsulated or entrapped in a polymer. In some embodiments, an implantable element includes cells, e.g., cells disposed within a polymeric enclosing component (e.g., alginate).

In some embodiments, an implantable element targets or is designed for a certain system of the body, e.g. the nervous system (e.g., peripheral nervous system (PNS) or central nervous system (CNS)), vascular system, skeletal system, respiratory system, endocrine system, lymph system, reproductive system, or gastrointestinal tract. In some embodiments, an implantable element is targeted to the CNS. In some embodiments, an implantable element targets or is designed for a certain part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

Components or materials used in an implantable element (or the entire implantable element) can be optimized for one or more of biocompatibility (e.g., it minimizes immune rejection or fibrosis; heat-resistance; elasticity; tensile strength; chemical resistance (e.g., resistance to oils, greases, disinfectants, bleaches, processing aids, or other chemicals used in the production, use, cleaning, sterilizing and disinfecting of the device); electrical properties; surface and volume conductivity or resistivity, dielectric strength; comparative tracking index; mechanical properties; shelf life, long term durability sterilization capability (e.g., capable of withstanding sterilization processes, such as steam, dry heat, ethylene oxide (EtO), electron beam, and/or gamma radiation, e.g., while maintaining the properties for the intended use of the device), e.g., thermal resistance to autoclave/steam conditions, hydrolytic stability for steam sterilization, chemical resistance to EtO, resistance to high-energy radiation (e.g., electron beam, UV, and gamma); or crystal structure.

An implantable element can be assembled in vivo (e.g., injectable substance that forms a structured shape in vivo, e.g., at body temperature) or ex vivo.

An implantable element can have nanodimensions, e.g., can comprise a nanoparticle, e.g., nanoparticle made of a polymer described herein, e.g., PLA. Nanoparticles can be chemically modified nanoparticles, e.g., modified to prevent uptake by macrophages and Kupfer cells (e.g., a process called opsonization); or to alter the circulation half-life of the nanoparticle.

Nanoparticles can include iron nanoparticle (injectable) (e.g., Advanced Magnetics iron nanoparticles). Exemplary nanoparticles are described in Veiseh et al (2010) Adv Drug Deliv Rev 62:284-304.

An implantable element can be configured for implantation in, administration to, or is administered to, implanted in or otherwise disposed into or onto any site of the body of a subject, including, but not limited to, the skin, a mucosal surface, a body cavity, intraperitoneal (IP) space, central nervous system (CNS) (e.g., brain or spinal cord), peripheral nervous system, an organ (e.g., heart, liver, kidney, bladder, pancreas, prostate, spleen, lung), lymphatic system, vasculature, oral cavity, nasal cavity, teeth, the gums, gastrointestinal tract, bone, hip, fat tissue (e.g., subcutaneous fat), muscle tissue, breast tissue, circulating blood, the eye, breast, vagina; uterus, a joint (e.g., in the knee, hip or spine): adjacent to a nerve, and a malignant or non-malignant tumor located on, in or near any of the foregoing.

In some embodiments, the implantable element is configured for implantation in, administration to, or is implanted or disposed into the IP space, e.g., within the peritoneal cavity, the omentumthe lesser sac. The lesser sac, also known as the omental bursa, refers to a cavity located in the abdomen formed by the omentum, and is in close proximity to, for example, the greater omentum, lesser omentum, stomach, small intestine, large intestine, liver, spleen, gastrosplenic ligament, adrenal glands, and pancreas. Typically, the lesser sac is connected to the greater sac via the omental foramen (i.e., the Foramen of Winslow). An implantable element may be implanted in or administered to the IP space, peritoneal cavity (e.g., the omentum, e.g., the lesser sac) or disposed on a surface within the peritoneal cavity (e.g., omentum, e.g., lesser sac) via injection or catheter. Additional considerations for implantation, administration or disposition of an implantable element into the omentum (e.g., the lesser sac) are provided in M. Pellicciaro et al. (2017) CellR4 5(3):e2410.

In some embodiments, the implantable element is configured for implantation in, administration to, or is implanted, administered or otherwise disposed into the CNS, e.g., the brain or spinal cord and their corresponding tissues and cavities, e.g., the dorsal body cavity, including the cranial cavity and the spinal canal. In some embodiments, the implantable element is configured for implantation in, administration to, or is implanted, administered to or otherwise disposed into an intracerebral space, e.g., the intraparenchymal space, the intraventricular space, or the subdural space. An implantable element may be implanted in the CNS or disposed on a surface within the CNS through a hole made in the skull and delivered via injection or catheter.

In some embodiments, the implantable element is configured for implantation in, administration to, or is implanted in, administered to or otherwise disposed into the eye, e.g., at one or more of the following: any surface or cavity within the eye, such as the retina, cornea, epithelium, aqueous humor, or vitreal space. An implantable element may be implanted in the eye or disposed on a surface within the eye through incision and/or injection.

An implantable element can comprise an electrochemical sensor, e.g., an electrochemical sensor including a working electrode and a reference electrode. For example, an electrochemical sensor includes a working electrode and a reference electrode that reacts with an analyte to generate a sensor measurement related to a concentration of the analyte in a fluid to which the eye-mountable device is exposed. The implantable element can comprise a window, e.g., of a transparent polymeric material having a concave surface and a convex surface a substrate, e.g., at least partially embedded in a transparent polymeric material. An implantable element can also comprise an electronics module including one or more of an antenna; and a controller electrically connected to the electrochemical sensor and the antenna, wherein the controller is configured to control the electrochemical sensor to obtain a sensor measurement related to a concentration of an analyte in a fluid to which the implantable element, e.g., an mountable implantable element is exposed and use the antenna to indicate the sensor measurement.

An implantable element may take any suitable shape, such as a sphere, spheroid, ellipsoid, disk, cylinder, torus, cube, stadiumoid, cone, pyramid, triangle, rectangle, square, or rod, or may comprise a curved or flat section. Any shaped, curved, or flat implantable element may be coated or chemically derivatized with a compound of Formula (II), a polymer modified with a compound of Formula (II), or a pharmaceutically acceptable salt thereof.

In some embodiments, an implantable element has a largest linear dimension (LLD), mean diameter or size that is 1 millimeter (mm) or smaller, or is within a range of 0.2 mm to 1 mm, e.g., any of 0.2, 0.3, 0.4, 0.5, 0.6, 0.7, 0.8, 0.9 or 1 mm. In some embodiments, an implantable element has an LLD, mean diameter or size that is greater than 0.5 mm, 1 mm, or 1.5 mm. In some embodiments, an implantable element described herein is in a size range of 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm. In some embodiments, the implantable element has an LLD, mean diameter or size of 0.5 mm to 1 mm or 1 mm to 4 mm.

In some embodiments, the implantable element has an LLD, mean diameter or size 1 mm to 2 mm. In some embodiments, the implantable element has a spherical shape and a mean diameter within any of the foregoing numerical ranges.

In some embodiments, an implantable element comprises at least one pore or opening, e.g., to allow for the free flow of materials. In some embodiments, the mean pore size of an implantable element is between about 0.1 μm to about 10 μm. For example, the mean pore size may be between 0.1 μm to 10 μm, 0.1 μm to 5 μm, 0.1 μm to 2 μm, 0.15 μm to 10 μm, 0.15 μm to 5 μm, 0.15 μm to 2 μm, 0.2 μm to 10 μm, 0.2 μm to 5 μm, 0.25 μm to 10 μm, 0.25 μm to 5 μm, 0.5 μm to 10 μm, 0.75 μm to 10 μm, 1 μm to 10 μm, 1 μm to 5 μm, 1 μm to 2 μm, 2 μm to 10 μm, 2 μm to 5 μm, or 5 μm to 10 μm. In some embodiments, the mean pore size of an implantable element is between about 0.1 μm to 10 μm. In some embodiments, the mean pore size of an implantable element is between about 0.1 μm to 5 μm. In some embodiments, the mean pore size of an implantable element is between about 0.1 μm to 1 μm.

In some embodiments, an implantable element is capable of preventing materials over a certain size from passing through a pore or opening. In some embodiments, an implantable element is capable of preventing materials greater than 50 kD, 75 kD, 100 kD, 125 kD, 150 kD, 175 kD, 200 kD, 250 kD, 300 kD, 400 kD, 500 kD, 750 kD, 1,000 kD from passing through.

An implantable element (e.g., an implantable element described herein) may be provided as a preparation or composition for implantation or administration to a subject. In some embodiments, at least 20%, 30%, 35%, 40%, 45%, 50%, 55%, 60%, 65%, 70%, 75%, 80%, 85%, 90%, 95% or 100% of the implantable elements in a preparation or composition have a characteristic as described herein, e.g., mean pore size.

In some embodiments, an implantable element may be configured for or used for varying periods of time, ranging from a few minutes to several years. For example, an implantable element may be configured for or used from about 1 hour to about 10 years. In some embodiments, an implantable element is configured for, or is used for, longer than about any of the following time periods: 1 to 24 hours; 1 to 7 days; 1 to 4 weeks; 1 to 24 months; 2 to 10 years, or longer. An implantable element may be configured to function for the expected duration of implantation, e.g., configured to resist inactivation by PFO for all or part of the expected duration.

In some embodiments, the implantable element is easily retrievable from a subject, e.g., without causing injury to the subject or without causing significant disruption of the surrounding tissue. In an embodiment, the implantable element can be retrieved with minimal or no surgical separation of the implantable element from surrounding tissue, e.g., via minimally invasive surgical insection, extraction, or resection.

In some embodiments, the implantable element is not an implantable element disclosed in any of WO2012/112982, WO2012/167223, WO2014/153126, WO2016/187225, WO2016/019391, WO2017/075630, WO 2017/075631, WO 2018/067615, WO 2019/169333, or US 2016-0030359.

In some embodiments, an implantable element is associated with a compound of Formula (II). In some embodiments, an implantable element is covalently modified with a compound of Formula (II). In some embodiments, an implantable element comprises a polymer modified with a compound of Formula (II). In some embodiments, an implantable element comprises a polymer modified with a compound of Formula (II) and a cell that is entirely or partially disposed within the implantable element.

In some embodiments, a surface of the implantable element comprising a cell (e.g., an engineered cell) is chemically modified with a compound of Formula (II). In some embodiments, a surface comprises an outer surface or an inner surface of the implantable element. In some embodiments, the surface (e.g., outer surface) of the implantable element comprising a cell (e.g., an engineered cell) is chemically modified with a compound of Formula (II). In some embodiments, the surface (e.g., outer surface) is covalently linked to a compound of Formula (II).

An implantable element may be coated with a compound of Formula (II) or a pharmaceutically acceptable salt thereof, or a polymer comprising a compound of Formula (II) or a pharmaceutically acceptable salt thereof. In an embodiment, the compound of Formula (II) is disposed on a surface, e.g., an inner or outer surface, of the implantable element. In some embodiments, the compound of Formula (II) is disposed on a surface, e.g., an inner or outer surface, of an enclosing component associated with an implantable element. In an embodiment, the compound of Formula (II) is distributed evenly across a surface. In an embodiment, the compound of Formula (II) is distributed unevenly across a surface.

In some embodiments, an implantable element (e.g., or an enclosing component thereof) is coated (e.g., covered, partially or in full), with a compound of Formula (II) or a polymer modified with a compound of Formula (II) or a pharmaceutically acceptable salt thereof. In some embodiments, an implantable element (e.g., or an enclosing component thereof) is coated with a single layer of a compound of Formula (II). In some embodiments, an implantable element is coated with multiple layers of a compound of Formula (II), e.g., at least 2 layers, 3 layers, 4 layers, 5 layers, 10 layers, 20 layers, 50 layers or more.

In an embodiment, a first portion of the surface of the implantable element comprises a compound of Formula (II) and a second portion of the implantable element lacks the compound, or has a substantially lower density of the compound.

In some embodiments, an implantable element is coated or chemically derivatized in a symmetrical manner with a compound of Formula (II), or a material comprising Formula (II), or a pharmaceutically acceptable salt thereof. In some embodiments, an implantable element is coated or chemically derivatized in an asymmetrical manner with a compound of Formula (II), or a polymer modified with a compound of Formula (II), or a pharmaceutically acceptable salt thereof. For example, an exemplary implantable element may be partially coated (e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated) with a compound of Formula (I) or a polymer modified with a compound of Formula (II) or a pharmaceutically acceptable salt thereof.

Exemplary implantable elements coated or chemically derivatized with a compound of Formula (II), or a polymer modified with a compound Formula (II), or a pharmaceutically acceptable salt thereof may be prepared using any method known in the art, such as through self-assembly (e.g., via block copolymers, adsorption (e.g., competitive adsorption), phase separation, microfabrication, or masking).

In some embodiments, the implantable element comprises a surface exhibiting two or more distinct physicochemical properties (e.g., 3, 4, 5, 6, 7, 8, 9, 10, or more distinct physicochemical properties).

In some embodiments, the coating or chemical derivatization of the surface of an exemplary implantable element with a compound of Formula (II), a polymer modified with a compound of Formula (II), or a pharmaceutically acceptable salt thereof is described as the average number of attached compounds per given area, e.g., as a density. For example, the density of the coating or chemical derivatization of an exemplary implantable element may be 0.01, 0.1, 0.5, 1, 5, 10, 15, 20, 50, 75, 100, 200, 400, 500, 750, 1,000, 2,500, or 5,000 compounds per square μm or square mm, e.g., on the surface or interior of said implantable element.

An implantable element comprising a compound of Formula (II) or a pharmaceutically acceptable salt thereof may have a reduced immune response (e.g., a marker of an immune response) compared to an otherwise identical implantable element that does not comprise a compound of Formula (I) or a pharmaceutically acceptable salt thereof. A marker of immune response is one or more of: PFO, cathepsin level or the level of a marker of immune response, e.g., TNF-α, IL-13, IL-6, G-CSF, GM-CSF, IL-4, CCL2, or CCL4, as measured, e.g., by ELISA. In some embodiments, the immune response to an implantable element comprising a compound of Formula (II) or a pharmaceutically acceptable salt thereof is reduced by at least about 1 percent and up to about 100 percent, e.g., about 5, 10, 15, 20, 25, 30, 35, 40, 45, 50, 55, 60, 65, 70, 75, 80, 85, 90, 95, or 99 percent. In some embodiments, the reduced immune response (e.g., a marker of an immune response) is measured after about any of 30 minutes, 1 hour, 6 hours, 12 hours, about any of 1 day, 2 days, 3 days or 4 days, about 1 week or 2 weeks, about any of 1 month, 2 months, 3 months, 6 months or longer. In some embodiments, an implantable element comprising a compound of Formula (II) is coated by the compound of Formula (II) or encapsulated in a layer (e.g., a polymeric layer) comprising a compound of Formula (II).

An implantable element may have a smooth surface, or may comprise a protuberance, depression, well, slit, or hole, or any combination thereof. Said protuberance, depression, well, slit or hole may be any size, e.g., from 10 μm to about 1 nm, about 5 μm to about 1 nm, about 2.5 μm to about 1 nm, 1 μm to about 1 nm, 500 nm to about 1 nm, or about 100 nm to about 1 nm. The smooth surface or protuberance, depression, well, slit, or hole, or any combination thereof, may be coated or chemically derivatized with a compound of Formula (II), polymer modified with a compound of Formula (II), or a pharmaceutically acceptable salt thereof.

In an embodiment, an implantable element comprises any of the polymers described herein, modified with a compound of Formula (II) or a pharmaceutically acceptable salt thereof.

In some embodiments, the implantable element (when comprising a compound of Formula II) comprises an increase in % N (as compared with an implantable element not comprising a compound of Formula II) of at least 0.1, 0.2, 0.5, 1.0, 1.5, 2, 2.5, 3, 3.5, 4, 4.5, 5, 5.5, 6, 6.5, 7, 7.5, 8, 8.5, 9, 9.5, or 10% N by weight, where % N is determined by elemental analysis and corresponds to the amount of compound of Formula II in the implantable element.

In some embodiments, the implantable element (when comprising a compound of Formula II) comprises an increase in % N (as compared with an implantable element not comprising a compound of Formula II) of 0.1 to 10% N by weight, where % N is determined by elemental analysis and corresponds to the amount of compound of Formula II in the implantable element.

In some embodiments, the implantable element (when comprising a compound of Formula II) comprises an increase in % N (as compared with an implantable element not comprising a compound of Formula II) of 0.1 to 2% N by weight, where % N is determined by elemental analysis and corresponds to the amount of compound of Formula II in the implantable element.

In some embodiments, the implantable element (when modified with a compound of Formula II) comprises an increase in % N (as compared with an implantable element not comprising a compound of Formula II) of 2 to 4% N by weight, where % N is determined by elemental analysis and corresponds to the amount of compound of Formula II in the implantable element.

In some embodiments, the implantable element (when comprising a compound of Formula II) comprises an increase in % N (as compared with an implantable element not comprising a compound of Formula II) of 4 to 8% N by weight, where % N is determined by elemental analysis and corresponds to the amount of compound of Formula II in the implantable element.

In some embodiments, the implantable element comprises between 5 to 50% of a compound of Formula (II), e.g., as measured using a quantative amine assay. In some embodiments, the implantable element comprises between 10 to 50% of a compound of Formula (II), e.g., 15 to 45% of a compound of Formula (II), 15 to 40% of a compound of Formula (II), 15 to 35% of a compound of Formula (II), 15 to 30% of a compound of Formula (II), 20 to 45% of a compound of Formula (II), 20 to 40% of a compound of Formula (II), 20 to 35% of a compound of Formula (II), or 20 to 30% of a compound of Formula (II), as measured using a quantative amine assay.

In some embodiments, an implantable element comprises an alginate (e.g., any of the alginates described herein) modified with a compound of Formula (II) (e.g., a compound of Formulas (II-a), (II-b), (II-c), (II-d), (II-e), (II-f), or a pharmaceutically acceptable salt thereof). In some embodiments, an implantable element comprises an alginate modified with a compound of Formula (II-a). In some embodiments, an implantable element comprises an alginate modified with a compound of Formula (II-b). In some embodiments, an implantable element comprises an alginate modified with a compound of Formula (II-c). In some embodiments, an implantable element comprises an alginate modified with a compound of Formula (II-d). In some embodiments, an implantable element comprises an alginate modified with a compound of Formula (II-e). In some embodiments, an implantable element comprises an alginate modified with a compound of Formula (II-f).

In some embodiments, an implantable element comprises an alginate modified with a compound shown in Table 2. In some embodiments, an implantable element comprises an alginate modified with Compound 400. In some embodiments, an implantable element comprises an alginate modified with Compound 401.

Cells and Therapeutic Agents

The implantable elements of the present disclosure may comprise a wide variety of different cell types (e.g., human cells), including but not limited to: adipose cells, epidermal cells, epithelial cells, endothelial cells, fibroblast cells, islet cells, mesenchymal stem cells, pericytes, subtypes of any of the foregoing, cells derived from any of the foregoing, cells derived from induced pluripotent stem cells and mixtures of one or more of any of the foregoing. Exemplary cell types include the cell types recited in WO 2017/075631 and WO 2019/195055. In an embodiment, the implantable elements described herein comprise a plurality of cells. In an embodiment, the plurality of cells is in the form of a cell suspension prior to being encapsulated within an implantable element described herein. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers. In some embodiments, the device does not comprise any islet cells and does not comprise any cells that are capable of producing insulin in a glucose-responsive manner.

The present invention features a cell that produces or is capable of producing a therapeutic agent for the prevention or treatment of a disease, disorder, or condition described herein. In an embodiment, the cell is an engineered cell. In an embodiment, the cell is engineered to sense a stimulus, e.g., a chemical signal, and express the therapeutic agent in response to the stimulus. The therapeutic agent may be any biological substance, such as a nucleic acid (e.g., a nucleotide, DNA, or RNA), a polypeptide, a lipid, a sugar (e.g., a monosaccharide, disaccharide, oligosaccharide, or polysaccharide), or a small molecule, each of which are further elaborated below. Exemplary therapeutic agents include the agents listed in WO 2017/075631 and WO 2019/195055.

In some embodiments, the cells (e.g., engineered cells) produce a nucleic acid. A nucleic acid produced by a cell described herein may vary in size and contain one or more nucleosides or nucleotides, e.g., greater than 2, 3, 4, 5, 10, 25, 50, or more nucleosides or nucleotides. In some embodiments, the nucleic acid is a short fragment of RNA or DNA, e.g., and may be used as a reporter or for diagnostic purposes. Exemplary nucleic acids include a single nucleoside or nucleotide (e.g., adenosine, thymidine, cytidine, guanosine, uridine monophosphate, inosine monophosphate), RNA (e.g., mRNA, siRNA, miRNA, RNAi), and DNA (e.g., a vector, chromosomal DNA). In some embodiments, the nucleic acid has an average molecular weight (in kD) of about 0.25, 0.5, 1, 1.5, 2, 2.5, 5, 10, 25, 50, 100, 150, 200 or more.

In some embodiments, the therapeutic agent is a peptide or polypeptide (e.g., a protein), such as a hormone, enzyme, cytokine (e.g., a pro-inflammatory cytokine or an anti-inflammatory cytokine), growth factor, clotting factor, or lipoprotein. A peptide or polypeptide (e.g., a protein, e.g., a hormone, growth factor, clotting factor or coagulation factor, antibody molecule, enzyme, cytokine, cytokine receptor, or a chimeric protein including cytokines or a cytokine receptor) produced by a cell in an implantable element can have a naturally occurring amino acid sequence, or may contain a variant of the naturally occurring sequence. The variant can be a naturally occurring or non-naturally occurring amino acid substitution, mutation, deletion or addition relative to the reference naturally occurring sequence. The naturally occurring amino acid sequence may be a polymorphic variant. The naturally occurring amino acid sequence can be a human or a non-human amino acid sequence. In some embodiments, the naturally occurring amino acid sequence or naturally occurring variant thereof is a human sequence. In addition, a peptide or polypeptide (e.g., a protein) for use with the present invention may be modified in some way, e.g., via chemical or enzymatic modification (e.g., glycosylation, phosphorylation). In some embodiments, the peptide has about 2, 3, 4, 5, 6, 7, 8, 9, 10, 12, 14, 16, 18, 20, 25, 30, 35, 40, 45, or 50 amino acids. In some embodiments, the protein has an average molecular weight (in kD) of 5, 10, 25, 50, 100, 150, 200, 250, 500 or more.

In some embodiments, the protein is a hormone. Exemplary hormones include anti-diuretic hormone (ADH), oxytocin, growth hormone (GH), prolactin, growth hormone-releasing hormone (GHRH), thyroid stimulating hormone (TSH), thyrotropin-release hormone (TRH), adrenocorticotropic hormone (ACTH), follicle-stimulating hormone (FSH), luteinizing hormone (LH), luteinizing hormone-releasing hormone (LHRH), thyroxine, calcitonin, parathyroid hormone, aldosterone, cortisol, epinephrine, glucagon, insulin, estrogen, progesterone, and testosterone. In some embodiments, the protein is insulin (e.g., insulin A-chain, insulin B-chain, or proinsulin). In some embodiments, the protein is a growth hormone, such as human growth hormone (hGH), recombinant human growth hormone (rhGH), bovine growth hormone, methione-human growth hormone, des-phenylalanine human growth hormone, and porcine growth hormone. In some embodiments, the protein is not insulin (e.g., insulin A-chain, insulin B-chain, or proinsulin).

In some embodiments, the protein is a growth factor, e.g., vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

In some embodiments, the protein is a clotting factor or a coagulation factor, e.g., a blood clotting factor or a blood coagulation factor. In some embodiments, the protein is a protein involved in coagulation, i.e., the process by which blood is converted from a liquid to solid or gel. Exemplary clotting factors and coagulation factors include Factor I (e.g., fibrinogen), Factor II (e.g., prothrombin), Factor III (e.g., tissue factor), Factor V (e.g., proaccelerin, labile factor), Factor VI, Factor VII (e.g., stable factor, proconvertin), Factor VIII (e.g., antihemophilic factor A), Factor VIIIC, Factor IX (e.g., antihemophilic factor B), Factor X (e.g., Stuart-Prower factor), Factor XI (e.g., plasma thromboplastin antecedent), Factor XII (e.g., Hagerman factor), Factor XIII (e.g., fibrin-stabilizing factor), von Willebrand factor, prekallikrein, heparin cofactor II, high molecular weight kininogen (e.g., Fitzgerald factor), antithrombin III, and fibronectin. In some embodiments, the protein is an anti-clotting factor, such as Protein C.

In some embodiments, the protein is an antibody molecule. As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full-length antibody which has an immunoglobulin Fc region). In an embodiment, an antibody molecule comprises a full-length antibody, or a full-length immunoglobulin chain. In an embodiment, an antibody molecule comprises an antigen binding or functional fragment of a full-length antibody, or a full-length immunoglobulin chain. In an embodiment, an antibody molecule is a monospecific antibody molecule and binds a single epitope, e.g., a monospecific antibody molecule having a plurality of immunoglobulin variable domain sequences, each of which binds the same epitope. In an embodiment, an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

Various types of antibody molecules may be produced by a cell in an implantable element described herein, including whole immunoglobulins of any class, fragments thereof, and synthetic proteins containing at least the antigen binding variable domain of an antibody. The antibody molecule can be an antibody, e.g., an IgG antibody, such as IgG₁, IgG₂, IgG₃, or IgG₄. An antibody molecule can be in the form of an antigen binding fragment including a Fab fragment, F(ab fragment, a single chain variable region, and the like. Antibodies can be polyclonal or monoclonal (mAb). Monoclonal antibodies may include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity. In some embodiments, the antibody molecule is a single-domain antibody (e.g., a nanobody). The described antibodies can also be modified by recombinant means, for example by deletions, additions or substitutions of amino acids, to increase efficacy of the antibody in mediating the desired function. Exemplary antibodies include anti-beta-galactosidase, anti-collagen, anti-CD14, anti-CD20, anti-CD40, anti-HER2, anti-IL-1, anti-IL-4, anti-IL6, anti-IL-13, anti-IL17, anti-IL18, anti-IL-23, anti-IL-28, anti-IL-29, anti-IL-33, anti-EGFR, anti-VEGF, anti-CDF, anti-flagellin, anti-IFN-α, anti-IFN-0, anti-IFN-γ, anti-mannose receptor, anti-VEGF, anti-TLR1, anti-TLR2, anti-TLR3, anti-TLR4, anti-TLR5, anti-TLR6, anti-TLR9, anti-PDF, anti-PD1, anti-PDL-1, or anti-nerve growth factor antibody. In some embodiments, the antibody is an anti-nerve growth factor antibody (e.g., fulranumab, fasinumab, tanezumab).

In some embodiments, the protein is a cytokine or a cytokine receptor, or a chimeric protein including cytokines or their receptors, including, for example tumor necrosis factor alpha and beta, their receptors and their derivatives, renin; lipoproteins; colchicine; corticotrophin; vasopressin; somatostatin; lypressin; pancreozymin; leuprolide; alpha-i-antitrypsin; atrial natriuretic factor; lung surfactant; a plasminogen activator other than a tissue-type plasminogen activator (t-PA), for example a urokinase; bombesin; thrombin; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-α and TGF-β, including TGF-01, TGF-02, TGF-03, TGF-04, or TGF-05; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (TLs), e.g., IL-1 to IL-10; superoxide dismutase; T-cell receptors; surface membrane proteins; decay accelerating factor; transport proteins; homing receptors; addressins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues. Suitable proteins or peptides may be native or recombinant and include, e.g., fusion proteins.

Examples of a polypeptide (e.g., a protein) produced by a cell in an implantable element described herein also include CCL1, CCL2 (MCP-1), CCL3 (MIP-1α), CCL4 (MIP-1β), CCL5 (RANTES), CCL6, CCL7, CCL8, CCL9 (CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1 (KC), CXCL2 (SDF 1a), CXCL3, CXCL4, CXCL5, CXCL6, CXCL7, CXCL8 (IL8), CXCL9, CXCL10, CXCL11, CXCL12, CXCL13, CXCL14, CXCL15, CXCL16, CXCL17, CX3CL1, XCL1, XCL2, TNFA, TNFB (LTA), TNFC (LTB), TNFSF4, TNFSF5 (CD40LG), TNFSF6, TNFSF7, TNFSF8, TNFSF9, TNFSF10, TNFSF11, TNFSF13B, EDA, IL2, IL15, IL4, IL13, IL7, IL9, IL21, IL3, IL5, IL6, IL11, IL27, IL30, IL31, OSM, LIF, CNTF, CTF1, IL12a, IL12b, IL23, IL27, IL35, IL14, IL16, IL32, IL34, IL10, IL22, IL19, IL20, IL24, IL26, IL29, IFNL1, IFNL2, IFNL3, IL28, IFNA1, IFNA2, IFNA4, IFNA5, IFNA6, IFNA7, IFNA8, IFNA10, IFNA13, IFNA14, IFNA16, IFNA17, IFNA21, IFNB1, IFNK, IFNW1, IFNG, ILlA (IL1F1), IL1B (IL1F2), IL1Ra (IL1F3), IL1F5 (IL36RN), IL1F6 (IL36A), IL1F7 (IL37), IL1F8 (IL36B), IL1F9 (IL36G), IL1F10 (IL38), IL33 (IL1F11), IL18 (IL1G), IL17, KITLG, IL25 (IL17E), CSF1 (M-CSF), CSF2 (GM-CSF), CSF3 (G-CSF), SPP1, TGFB1, TGFB2, TGFB3, CCL3L1, CCL3L2, CCL3L3, CCL4L1, CCL4L2, IL17B, IL17C, IL17D, IL17F, AIMP1 (SCYE1), MIF, Areg, BC096441, Bmp1, Bmp10, Bmp15, Bmp2, Bmp3, Bmp4, Bmp5, Bmp6, Bmp7, Bmp8a, Bmp8b, Clqtnf4, Ccl21a, Ccl27a, Cd70, Cerl, Cklf, Clcfl, Cmtm2a, Cmtm2b, Cmtm3, Cmtm4, Cmtm5, Cmtm6, Cmtm7, Cmtm8, Crlfl, Ctf2, Ebi3, Edn1, Fam3b, Fasl, Fgf2, Flt31, Gdf10, Gdf11, Gdf15, Gdf2, Gdf3, Gdf5, Gdf6, Gdf7, Gdf9, Gm12597, Gm13271, Gm13275, Gm13276, Gm13280, Gm13283, Gm2564, Gpi1, Grem1, Grem2, Grn, Hmgb1, Ifna11, Ifna12, Ifna9, Ifnab, Ifne, II17a, II23a, 1125, 1131, Iltifb, Inhba, Lefty1, Lefty2, Mstn, Nampt, Ndp, Nodal, Pf4, Pglyrpl, Pr17d1, Scg2, Scgb3a1, Slurp1, Spp1, Thpo, Tnfsf10, Tnfsf11, Tnfsf12, Tnfsf13, Tnfsf13b, Tnfsf14, Tnfsf15, Tnfsf18, Tnfsf4, Tnfsf8, Tnfsf9, Tslp, Vegfa, Wnt1, Wnt2, Wnt5a, Wnt7a, Xcll, epinephrine, melatonin, triiodothyronine, a prostaglandin, a leukotriene, prostacyclin, thromboxane, islet amyloid polypeptide, müllerian inhibiting factor or hormone, adiponectin, corticotropin, angiotensin, vasopressin, arginine vasopressin, atriopeptin, brain natriuretic peptide, calcitonin, cholecystokinin, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, hepcidin, human chorionic gonadotropin, human placental lactogen, inhibin, somatomedin, leptin, lipotropin, melanocyte stimulating hormone, motilin, orexin, oxytocin, pancreatic polypeptide, pituitary adenylate cyclase-activating peptide, relaxin, renin, secretin, somatostatin, thrombopoietin, thyrotropin, thyrotropin-releasing hormone, vasoactive intestinal peptide, androgen, alpha-glucosidase (also known as acid maltase), glycogen phosphorylase, glycogen debrancher enzyme, phosphofructokinase, phosphoglycerate kinase, phosphoglycerate mutase, lactate dehydrogenase, carnitine palymityl transferase, carnitine, and myoadenylate deaminase.

In some embodiments, the protein is a replacement therapy or a replacement protein. In some embodiments, the replacement therapy or replacement protein is a clotting factor or a coagulation factor, e.g., Factor VIII (e.g., comprises a naturally occurring human Factor VIII amino acid sequence or a variant thereof) or Factor IX (e.g., comprises a naturally occurring human Factor IX amino acid sequence or a variant thereof).

In some embodiments, the cell is engineered to express a Factor VIII, e.g., a recombinant Factor VIII. In some embodiments, the cell is derived from human tissue and is engineered to express a Factor VIII, e.g., a recombinant Factor VIII. In some embodiments, the recombinant Factor VIII is a B-domain-deleted recombinant Factor VIII (FVIII-BDD).

In some embodiments, the cell is derived from human tissue and is engineered to express a Factor IX, e.g., a recombinant Factor IX. In some embodiments, the cell is engineered to express a Factor IX, e.g., a wild-type human Factor IX (FIX), or a polymorphic variant thereof.

In some embodiments, the cell is engineered to express a gain-in-function (GIF) variant of a wild-type FIX protein (FIX-GIF), wherein the GIF variant has higher specific activity than the corresponding wild-type FIX.

In some embodiments, the replacement therapy or replacement protein is an enzyme, e.g., alpha-galactosidase, alpha-L-iduronidase (IDUA), or N-sulfoglucosamine sulfohydrolase (SGSH). In some embodiments, the replacement therapy or replacement protein is an enzyme, e.g., an alpha-galactosidase A (e.g., comprises a naturally-occurring human alpha-galactosidase A amino acid sequence or a variant thereof). In some embodiments, the replacement therapy or replacement protein is a cytokine or an antibody.

In some embodiments, the therapeutic agent is a sugar, e.g., monosaccharide, disaccharide, oligosaccharide, or polysaccharide. In some embodiments, a sugar comprises a triose, tetrose, pentose, hexose, or heptose moiety. In some embodiments, the sugar comprises a a linear monosaccharide or a cyclized monosaccharide. In some embodiments, the sugar comprises a glucose, galactose, fructose, rhamnose, mannose, arabinose, glucosamine, galactosamine, sialic acid, mannosamine, glucuronic acid, galactosuronic acid, mannuronic acid, or guluronic acid moiety. In some embodiments, the sugar is attached to a protein (e.g., an N-linked glycan or an O-linked glycan). Exemplary sugars include glucose, galactose, fructose, mannose, rhamnose, sucrose, ribose, xylose, sialic acid, maltose, amylose, inulin, a fructooligosaccharide, galactooligosaccharide, a mannan, a lectin, a pectin, a starch, cellulose, heparin, hyaluronic acid, chitin, amylopectin, or glycogen. In some embodiments, the therapeutic agent is a sugar alcohol.

In some embodiments, the therapeutic agent is a lipid. A lipid may be hydrophobic or amphiphilic, and may form a tertiary structure such as a liposome, vesicle, or membrane or insert into a liposome, vesicle, or membrane. A lipid may comprise a fatty acid, glycerolipid, glycerophospholipid, sterol lipid, prenol lipid, sphingolipid, saccharolipid, polyketide, or sphingolipid. Examples of lipids produced by a cell described herein include anandamide, docosahexaenoic acid, aprostaglandin, a leukotriene, a thromboxane, an eicosanoid, a triglyceride, a cannabinoid, phosphatidylcholine, phosphatidylethanolamine, a phosphatidylinositol, a phosohatidic acid, a ceramide, a sphingomyelin, a cerebroside, a ganglioside, estrogen, androsterone, testosterone, cholesterol, a carotenoid, a quinone, a hydroquinone, or a ubiquinone.

In some embodiments, the therapeutic agent is a small molecule. A small molecule may include a natural product produced by a cell. In some embodiments, the small molecule has poor availability or does not comply with the Lipinski rule of five (a set of guidelines used to estimate whether a small molecule will likely be an orally active drug in a human; see, e.g., Lipinski, C. A. et al (2001) Adv Drug Deliv 46:2-36). Exemplary small molecule natural products include an anti-bacterial drug (e.g., carumonam, daptomycin, fidaxomicin, fosfomycin, ispamicin, micronomicin sulfate, miocamycin, mupiocin, netilmicin sulfate, teicoplanin, thienamycin, rifamycin, erythromycin, vancomycin), an anti-parasitic drug (e.g., artemisinin, ivermectin), an anticancer drug (e.g., doxorubicin, aclarubicin, aminolaevulinic acid, arglabin, omacetaxine mepesuccinate, paclitaxel, pentostatin, peplomycin, romidepsin, trabectdin, actinomycin D, bleomycin, chromomycin A, daunorubicin, leucovorin, neocarzinostatin, streptozocin, trabectedin, vinblastine, vincristine), anti-diabetic drug (e.g., voglibose), a central nervous system drug (e.g., L-dopa, galantamine, zicontide), a statin (e.g., mevastatin), an anti-fungal drug (e.g., fumagillin, cyclosporin), 1-deoxynojirimycin, and theophylline, sterols (cholesterol, estrogen, testosterone). Additional small molecule natural products are described in Newman, D. J. and Cragg, M. (2016) J Nat Prod 79:629-661 and Butler, M. S. et al (2014) Nat Prod Rep 31:1612-1661.

In some embodiments, the cell is engineered to synthesize a non-protein or non-peptide small molecule. For example, in an embodiment a cell can produce a statin (e.g., taurostatin, pravastatin, fluvastatin, or atorvastatin).

In some embodiments, the therapeutic agent is an antigen (e.g., a viral antigen, a bacterial antigen, a fungal antigen, a plant antigen, an environmental antigen, or a tumor antigen). An antigen is recognized by those skilled in the art as being immunostimulatory, i.e., capable of stimulating an immune response or providing effective immunity to the organism or molecule from which it derives. An antigen may be a nucleic acid, peptide, protein, sugar, lipid, or a combination thereof.

The cells, e.g., engineered cells, e.g., engineered cells described herein, may produce a single therapeutic agent or a plurality of therapeutic agents. In some embodiments, the cells produce a single therapeutic agent. In some embodiments, a cluster of cells comprises cells that produce a single therapeutic agent. In some embodiments, at least about 1 percent, or about 5, 10, 20, 25, 30, 40, 50, 60, 70, 80, 90, 95, or 99 percent of the cells in a cluster produce a single therapeutic agent (e.g., a therapeutic agent described herein). In some embodiments, the cells produce a plurality of therapeutic agents, e.g., at least 2, 3, 4, 5, 6, 7, 8, 9, or 10 therapeutic agents. In some embodiments, a cluster of cells comprises cells that produce a plurality of therapeutic agents. In some embodiments, at least about 1 percent, or about 5, 10, 20, 25, 30, 40, 50, 60, 70, 80, 90, 95, or 99 percent of the cells in a cluster produce a plurality of therapeutic agents (e.g., a therapeutic agent described herein).

The therapeutic agents may be related or may form a complex. In some embodiments, the therapeutic agent secreted or released from a cell in an active form. In some embodiments, the therapeutic agent is secreted or released from a cell in an inactive form, e.g., as a prodrug. In the latter instance, the therapeutic agent may be activated by a downstream agent, such as an enzyme. In some embodiments, the therapeutic agent is not secreted or released from a cell, but is maintained intracellularly. For example, the therapeutic agent may be an enzyme involved in detoxification or metabolism of an unwanted substance, and the detoxification or metabolism of the unwanted substance occurs intracellularly.

Methods of Treatment

Described herein are methods for preventing or treating a disease, disorder, or condition in a subject through administration or implantation of an implantable element comprising a compound of Formula (II) or a pharmaceutically acceptable salt thereof. In some embodiments, the methods described herein directly or indirectly reduce or alleviate at least one symptom of a disease, disorder, or condition. In some embodiments, the methods described herein prevent or slow the onset of a disease, disorder, or condition. In some embodiments, the subject is a human.

In some embodiments, the disease, disorder, or condition affects a system of the body, e.g. the nervous system (e.g., peripheral or central nervous system), vascular system, skeletal system, respiratory system, endocrine system, lymph system, reproductive system, or gastrointestinal tract. In some embodiments, the disease, disorder, or condition affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

In some embodiments, the disease, disorder or condition is a neurodegenerative disease, diabetes (Type 1 or Type 2), a heart disease, an autoimmune disease, a cancer, a liver disease, a lysosomal storage disease, a blood clotting disorder or a coagulation disorder, an orthopedic condition, an amino acid metabolism disorder.

In some embodiments, the disease, disorder or condition is a neurodegenerative disease. Exemplary neurodegenerative diseases include Alzheimer© disease, Huntington [symbol] disease, Parkinson [symbol] disease (PD) amyotrophic lateral sclerosis (ALS), multiple sclerosis (MS) and cerebral palsy (CP), dentatorubro-pallidoluysian atrophy (DRPLA), neuronal intranuclear hyaline inclusion disease (NIHID), dementia with Lewy bodies, Down s syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Jakob-Creutzfeldt disease, Niemann-Pick disease type 3, progressive supranuclear palsy, subacute sclerosing panencephalitis, spinocerebellar ataxias, Pick© disease, and dentatorubral-pallidoluysian atrophy.

In some embodiments, the disease, disorder, or condition is an autoimmune disease, e.g., scleroderma, multiple sclerosis, lupus, or allergies.

In some embodiments, the disease is a liver disease, e.g., hepatitis B, hepatitis C, cirrhosis, NASH.

In some embodiments, the disease, disorder, or condition is cancer. Exemplary cancers include leukemia, lymphoma, melanoma, lung cancer, brain cancer (e.g., glioblastoma), sarcoma, pancreatic cancer, renal cancer, liver cancer, testicular cancer, prostate cancer, or uterine cancer.

In some embodiments, the disease, disorder, or condition is an orthopedic condition. Exemplary orthopedic conditions include osteoporosis, osteonecrosis, Paget's disease, or a fracture.

In some embodiments, the disease, disorder or condition is a lysosomal storage disease. Exemplary lysosomal storage diseases include Gaucher disease (e.g., Type I, Type II, Type III), Tay-Sachs disease, Fabry disease, Farber disease, Hurler syndrome (also known as mucopolysaccharidosis type I (MPS I)), Hunter syndrome, lysosomal acid lipase deficiency, Niemann-Pick disease, Salla disease, Sanfilippo syndrome (also known as mucopolysaccharidosis type IIIA (MPS3A)), multiple sulfatase deficiency, Maroteaux-Lamy syndrome, metachromatic leukodystrophy, Krabbe disease, Scheie syndrome, Hurler-Scheie syndrome, Sly syndrome, hyaluronidase deficiency, Pompe disease, Danon disease, gangliosidosis, or Morquio syndrome.

In some embodiments, the disease, disorder, or condition is a blood clotting disorder or a coagulation disorder. Exemplary blood clotting disorders or coagulation disorders include hemophilia (e.g., hemophilia A or hemophilia B), Von Willebrand diaease, thrombocytopenia, uremia, Bernard-Soulier syndrome, Factor XII deficiency, vitamin K deficiency, or congenital afibrinogenimia.

In some embodiments, the disease, disorder, or condition is an amino acid metabolism disorder, e.g., phenylketonuria, tyrosinemia (e.g., Type 1 or Type 2), alkaptonuria, homocystinuria, hyperhomocysteinemia, maple syrup urine disease.

In some embodiments, the disease, disorder, or condition is a fatty acid metabolism disorder, e.g., hyperlipidemia, hypercholesterolemia, galactosemia.

In some embodiments, the disease, disorder, or condition is a purine or pyrimidine metabolism disorder, e.g., Lesch-Nyhan syndrome.

In some embodiments, the disease, disorder, or condition is not Type I diabetes and/or is not Type II diabetes.

The present invention further comprises methods for identifying a subject having or suspected of having a disease, disorder, or condition described herein, and upon such identification, administering to the subject implantable element comprising a cell, e.g., optionally encapsulated by an enclosing component, and optionally modified with a compound of Formula (II) as described herein, or a composition thereof. In an embodiment, the subject is a human.

### EXAMPLES

In order that the invention described herein may be more fully understood, the following examples are set forth. The synthetic and biological examples described in this application are offered to illustrate the compounds, compositions, devices, and methods provided herein and are not to be construed in any way as limiting their scope.

The compounds, modified polymers, implantable elements, and compositions thereof provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Exemplary compounds, modified polymers, implantable elements, and compositions of the invention may be prepared using any of the strategies described below.

### Example 1: Synthesis of Exemplary Compounds

General Protocols

The procedures below describe methods of preparing exemplary compounds for preparation of chemically modified implantable elements. The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Huisgen cycloaddition to afford 1,4-substituted triazoles

The copper-catalyzed Huisgen [3+2] cycloaddition was used to prepare triazole-based compounds and compositions, devices, and materials thereof. The scope and typical protocols have been the subject of many reviews (e.g., Meldal, M. and Tornoe, C. W. Chem. Rev. (2008) 108:2952-3015; Hein, J. E. and Fokin, V. V. Chem. Soc. Rev. (2010) 39(4):1302-1315).

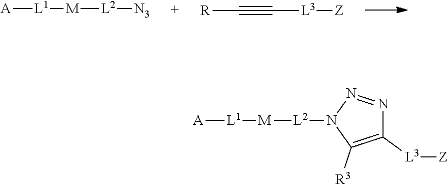

In the example shown above, the azide is the reactive moiety in the fragment containing the connective element A, while the alkyne is the reactive component of the pendant group Z. As depicted below, these functional handles can be exchanged to produce a structurally related triazole product. The preparation of these alternatives is similar, and do not require special considerations.

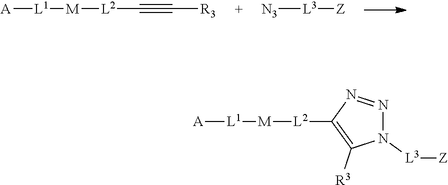

A typical Huisgen cycloaddition procedure starting with an iodide is outlined below. In some instances, iodides are transformed into azides during the course of the reaction for safety.

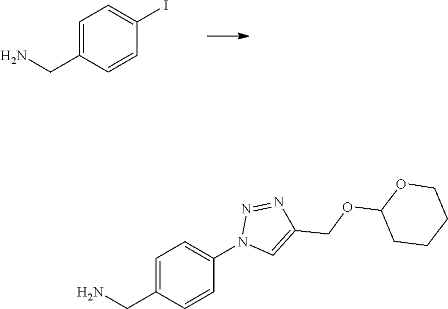 [SMILES: NCc1ccc(I)cc1 ; NCc1ccc(-n2cc(COC3CCCCO3)nn2)cc1]

A solution of sodium azide (1.1 eq), sodium ascorbate, (0.1 eq) trans-N,N′-dimethylcyclohexane-1,2-diamine (0.25 eq), copper (I) iodide in methanol (1.0 M) was degassed with bubbling nitrogen and treated with the acetylene (1 eq) and the aryl iodide (1.2 eq). This mixture was stirred at room temperature for 5 minutes, then warmed to 55° C. for 16 h. The reaction was then cooled to room temperature, filtered through a funnel, and the filter cake washed with methanol. The combined filtrates were concentrated and purified via flash chromatography on silica gel (120 g silica, gradient of 0 to 40% (3% aqueous ammonium hydroxide, 22% methanol, remainder dichloromethane) in dichloromethane to afford the desired target material.

A typical Huisgen cycloaddition procedure starting with an azide is outlined below.

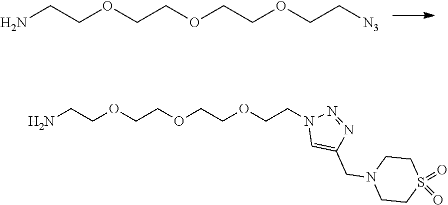 [SMILES: [N-]=[N+]=NCCOCCOCCOCCN ; NCCOCCOCCOCCn1cc(CN2CCS(=O)(=O)CC2)nn1]

A solution of tris[(1-benzyl-1H-1,2,3-triazol-4-yl)methyl]amine (0.2 eq), triethylamine (0.5 eq), copper (I) iodide (0.06 eq) in methanol (0.4 M, limiting reagent) was treated with the acetylene (1.0 eq) and cooled to 0° C. The reaction was allowed to warm to room temperature over 30 minutes, then heated to 55° C. for 16 h. The reaction was cooled to room temperature, concentrated, and purified with HPLC (C18 column, gradient of 0 to 100% (3% aqueous ammonium hydroxide, 22% methanol remainder dichloromethane) in dichloromethane to afford the desired target material.

### Huisgen cycloaddition to afford 1,5-substituted triazoles

The Huisgen [3+2] cycloaddition was also performed with ruthenium catalysts to obtain 1,5-disubstituted products preferentially (e.g., as described in Zhang et al, J. Am. Chem. Soc., 2005, 127, 15998-15999; Boren et al, J. Am. Chem. Soc., 2008, 130, 8923-8930).

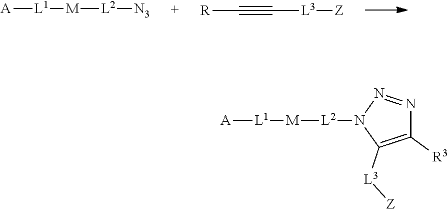

As described previously, the azide and alkyne groups may be exchanged to form similar triazoles as depicted below.

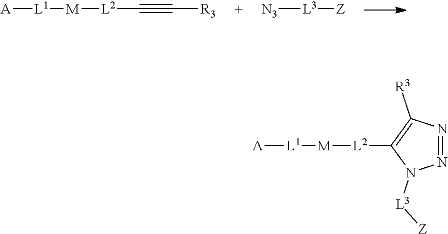

A typical procedure is described as follows: a solution of the alkyne (1 eq) and the azide (1 eq) in dioxane (0.8M) were added dropwise to a solution of pentamethylcyclo-pentadienylbis(triphenylphosphine) ruthenium(II) chloride (0.02 eq) in dioxane (0.16M). The vial was purged with nitrogen, sealed and the mixture heated to 60° C. for 12 h. The resulting mixture was concentrated and purified via flash chromatography on silica gel to afford the requisite compound.

### Synthesis of Compound 301

### Step 1: Synthesis of 3-methylsulfonyl-1-prop-2-ynyl-azetidine

A suspension of sodium hydride (6.70 g, 174.78 mmol) was stirred in THE (300 mL) at 0° C. 3-methylsulfonylazetidine hydrochloride (15 g, 87.39 mmol) was added and stirred for 30 min under N₂. 3-bromoprop-1-yne (12.99 g, 87.39 mmol, 9.70 mL, 80% purity) was then added dropwise. The reaction mixture was stirred from 0° C. to room temperature overnight, then any solid formed was filtered through a Buchner funnel and washed with methylene chloride (50 mL×3). The filtrate was concentrated, and residues were re-dissolved in methylene chloride (200 mL). Celite (˜20 g) was added and the solvent was removed by rotary evaporation. Dried powder was loaded on a 65 g of ISCO sample cartridge, and the crude product was purified on a 330 g of ISCO silica gel column, eluting with 0-5% of MeOH/CH₂Cl₂. Pure product was collected by TLC (10% MeOH/CH₂Cl₂, iodine stain) guidance to afford 8.08 g (53.37%) of title product as a wax. ¹H NMR (400 MHz, Chloroform-d) δ 3.83 (dq, J=8.6, 5.9 Hz, 1H), 3.76-3.70 (m, 2H), 3.66 (dd, J=8.5, 5.8 Hz, 2H), 3.32 (d, J=2.4 Hz, 2H), 2.94 (s, 3H), 2.32 (t, J=2.4 Hz, 1H).

LCMS m/z: [M+H]⁺ Calcd for C7H11NSO2, 174.0583; Observed, 174.058.

### Step 2: Synthesis of 2-[2-[2-[2-[4-[(3-methylsulfonylazetidin-1-yl)methyl]triazol-1-yl]ethoxy]ethoxy]ethoxy]ethanamine

3-Methylsulfonyl-1-prop-2-ynyl-azetidine (8.05 g, 46.47 mmol) was dissolved in methanol (250 mL) at rt with stirring under N2. Tris[(1-benzyl-1H-1,2,3-triazol-4-yl)methyl]-amine (5.42 g, 10.22 mmol), triethylamine (1.18 g, 11.62 mmol, 1.62 mL) and copper (I) iodide (885.01 mg, 4.65 mmol) were subsequently added, and the suspension was cooled to 0° C. 2-[2-[2-(2-azidoethoxy)ethoxy]ethoxy]ethanamine (11.87 g, 54.37 mmol, 10.79 mL) was added dropwise. After addition was completed, the ice bath was removed and the reaction mixture was warmed up to room temperature then heated at 55° C. overnight. The methanol was removed by rotary evaporation, and the residue was re-dissolved in methylene chloride (200 mL). Celite (˜20 g) was added and the solvent was removed by rotary evaporation. Dried powder was loaded at the top of a silica gel column, eluting with 0-35% of ultra (a mixed solvent of 3 L CH₂Cl₂, 880 mL MeOH and 120 mL NH₄OH)/CH₂Cl₂ to afford 11.7 g (64.31%) of title product as a viscous oil. ¹H NMR (500 MHz, Chloroform-d) δ 7.63 (s, 1H), 4.50 (t, J=5.1 Hz, 2H), 3.90-3.81 (m, 3H), 3.75 (s, 2H), 3.68-3.60 (m, 4H), 3.58 (d, J=3.0 Hz, 9H), 3.49 (t, J=5.2 Hz, 2H), 2.85 (d, J=6.1 Hz, 5H). ¹³C NMR (126 MHz, Chloroform-d) δ 143.29, 123.33, 72.93, 70.51, 70.45, 70.21, 69.44, 53.53, 52.61, 51.96, 50.22, 41.59, 38.45. LCMS m/z: [M+H]⁺ Calcd for C15H29O5N5S, 392.1962; Observed, 392.1967.

### Synthesis of Compound 300

### Step 1: 4-(3-chloroprop-2-yn-1-yl)thiomorpholine 1,1-dioxide

To a 500 mL of two-sided round bottom flask, 4-propargylthiomorpholine 1,1-dioxide (10 g, 57.73 mmol, 3.17 mL) and anhydrous methylene chloride (200 mL) were charged. The solution was stirred at −78° C. under N₂, and a solution of n-butyllithium (1.6 M in hexane; 86.59 mmol, 54.12 mL) was added dropwise through an additional funnel. After 1 hour stirring, N-chlorosuccinimide (98%, 11.56 g, 86.59 mmol) was added. The reaction mixture was stirred from −78° C. to room temperature overnight, then quenched by saturated NH₄Cl (125 mL). The aqueous layer was extracted with methylene chloride (200 mL×2), and the combined organic phases were dried over MgSO₄, filtered and evaporated onto Celite (˜30 g). Dried powder was packed into a silica gel column, eluting with methylene chloride. Peaks were monitored with ELSD and 214 nm using TLC (9 mL of CH₂Cl₂ plus 0.15 mL of 10% MeOH/CH₂Cl₂, iodine stain) against starting material guided product collection. 5.35 g (44.63%) of title product was obtained as an off white solid. ¹H NMR (500 MHz, Chloroform-d) δ 3.43 (s, 2H), 3.14-3.04 (m, 8H). LCMS m/z: [M+H]⁺ Calcd for C7H10NSClO2, 208.0194; Observed, 208.0196.

### Step 2: Synthesis of tert-butyl N-[2-[2-[2-[2-[5-Chloro-4-[(1,1-dioxo-1,4-thiazinan-4-yl)methyl]triazol-1-yl]ethoxy]ethoxy]ethoxy]ethyl]carbamate

To a 200 mL of pressure vial were added 4-(3-chloroprop-2-yn-1-yl)thiomorpholine 1,1-dioxide (5.35 g, 25.76 mmol), tert-butyl N-[2-[2-[2-(2-azidoethoxy)ethoxy]ethoxy]ethyl]carbamate (20.50 g, 64.40 mmol) and toluene (50 mL). The vial was sealed. Reaction mixture was heated to 120° C. with stirring overnight and cooled to it. Toluene was removed on a rotary evaporator. Residues were re-dissolved in methylene chloride (150 mL) and concentrated onto Celite (˜25 g). Dried powder was purified on a 330 g of ISCO silica gel column, eluting with 0-5% of MeOH/DCM. Two regioisomeres were separated by using TLC (5% of MeOH/DCM, iodine stain) guidance. 7.56 g (55.79%) of title product was obtained as a viscous oil. ¹H NMR (400 MHz, Chloroform-d) δ 5.02 (s, 1H), 4.51 (t, J=5.7 Hz, 2H), 3.96 (t, J=5.7 Hz, 2H), 3.79 (s, 2H), 3.59 (d, J=8.0 Hz, 8H), 3.52 (t, J=5.2 Hz, 2H), 3.30 (q, J=5.5 Hz, 2H), 3.07 (s, 8H), 1.43 (s, 9H). ¹³C NMR (126 MHz, Chloroform-d) δ 155.98, 138.67, 125.40, 79.22, 70.67, 70.58, 70.55, 70.23, 70.21, 68.78, 51.43, 50.68, 50.40, 48.08, 40.37, 28.44. LCMS m/z: [M+H]⁺ Calcd for C20H36O7N5SCl, 526.2097; Observed, 526.2097.

### Step 3: Synthesis of 2-[2-[2-[2-[5-Chloro-4-[(1,1-dioxo-1,4-thiazinan-4-yl) methyl]triazol-1-yl]ethoxy]ethoxy]ethoxy]ethanamine

tert-Butyl N-[2-[2-[2-[2-[5-chloro-4-[(1,1-dioxo-1,4-thiazinan-4-yl)methyl]triazol-1-yl]ethoxy]ethoxy]ethoxy]ethyl]carbamate (6.4 g, 12.17 mmol) was dissolved in 1,4-dioxane (40 mL) at room temperature with stirring. 2M HCl in diethyl ether (50 mmol, 25 mL) was slowly added, and the reaction mixture was heated at 55° C. overnight. The solvents were evaporated on a rotary evaporator, and the remaining solid was stirred in water (50 mL) and methylene chloride (50 mL). The organic phase was discarded, and the aqueous phase was basified with 6 N NaOH to pH-8-9, The product was extracted by methylene chloride (50 mL×4), and the combined organic phases were dried over MgSO₄ then filtered. The filtrate was concentrated and dried to afford 5.02 g (96.87%) of title product as a viscous oil. ¹H NMR (400 MHz, Chloroform-d) δ 4.55 (t, J=5.6 Hz, 2H), 3.97 (t, J=5.6 Hz, 2H), 3.81 (s, 2H), 3.66-3.59 (m, 8H), 3.56 (t, J=5.2 Hz, 2H), 3.09 (s, 8H), 2.93 (t, J=5.2 Hz, 2H). ¹³C NMR (126 MHz, Chloroform-d) δ 138.67, 125.42, 73.27, 70.65, 70.60, 70.50, 70.22, 68.75, 51.39, 50.64, 50.38, 48.08, 41.66. LCMS m/z: [M+H]⁺ Calcd for C15H28O5N5SCl, 426.1572; Observed, 426.1567.

Exemplary Protocol for Preparing Methacrylamides

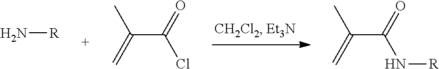

Each amine described herein may be converted to the corresponding methacrylamide following the general protocol outlined above. Generally, a solution of the free amine and triethylamine (1.2 eq) in CH₂Cl₂ (100 mL) was cooled to 0° C. in an ice-bath and methacryloyl chloride (1.99 mL, 20.42 mmol, 1.1 eq) was added in a dropwise fashion. The reaction was stirred overnight, following by slowly warming to room temperature after 24 hours. 20 grams of Celite were added and the solvent was removed under reduced pressure. The residue was purified by silica gel chromatography (220 g) using dichloromethane/methanol as the mobile phase to afford the desired methacrylamide compound, which was confirmed by LCMS.

### Example 2: Conjugation of Exemplary Compounds to Polymers

Exemplary compounds may be attached to a polymer to prepare a modified polymer. In this example, compounds of the disclosure were conjugated to alginate, a polymer comprising reactive carboxylic acid groups. Any of the components capable of coupling to a carboxylic acid, such as an amine described herein, may be an appropriate partner for this coupling reaction.

Compounds 300 and 301 were conjugated to alginate using the method outlined herein. The alginate polymer was dissolved in water (30 mL/gram alginate) and treated with 2-chloro-4,6-dimethoxy-1,3,5-triazine (0.5 eq) and N-methylmorpholine (1 eq). The compound of interest (one of Compound 200-218) was then dissolved in acetonitrile (0.3M) and added to the alginate solution. The reaction was then warmed to 55° C. for 16 h, cooled to room temperature, concentrated via rotary evaporation, then dissolved in water. The mixture was then filtered through a bed of cyano-modified silica gel (Silicycle) and the filter cake was washed with water. The resulting solution was then dialyzed (10,000 MWCO membrane) against water for 24 hours, replacing the water twice. The resulting solution was concentrated via lyophilization to afford the functionalized alginate.

### Example 3: Conjugation of Exemplary Compounds to NHS-Modified Plates

Exemplary compounds of the invention were prepared at a concentration of 0.1M in a 0.1M bicarbonate buffer (pH 8.2) containing 25% v/v dimethylsulfoxide (DMSO). Control solutions of 0.1M PEG750-amine and 0.01% fibronectin were prepared in 0.1M bicarbonate buffer (pH 8.2).

Each small molecule amine solution (100 μL) was pipetted into eight wells of an NHS-activated 96 well plate and incubated 2 hours at room temperature. Each plate consisted of two lanes containing the two control solutions and ten lanes containing the test molecule solutions. The test wells were rinsed once with 200 μL 0.1M bicarbonate buffer (pH 8.2) containing 25% v/v DMSO followed by three washes with 200 μL Hyclone™ water. The control wells were rinsed with 0.1M bicarbonate buffer (pH 8.2) followed by three 200 μL Hyclone™ water washes. Plates were dried at room temperature in a sterile hood and stored at 4-8° C. until use.

### Example 4: Conjugation of Exemplary Compounds to Silicone Disks

Disks (5 mm) were cut from a medical grade silicone sheet (1 mm thick) using a biopsy punch. Disks were rinsed several times with HyClone water to remove particulates and then cleaned by sonication: 10 minutes each in 200 proof ethanol, acetone, and hexane. Cleaned disks were dried overnight under vacuum. Small molecule methacrylamides (e.g., compounds of Formula (I) described herein) were screened for their solubility at 0.2M in blends of DMSO and toluene. Fresh solutions of the appropriate DMSO/toluene blend (typically 5-15 v/v % DMSO) were prepared the day of the reaction and degassed with nitrogen prior to use. The methacrylamide was added and vortexed or sonicated to achieve a clear 0.2M working solution. The surface of clean PDMS disks were activated by air plasma treatment (<300 mtorr, 30 W, 1 minute per side). After the second treatment, the disks were immediately removed from the reactor and transferred to the working solution for a one-hour reaction with mild agitation. Post-reaction, the disks were washed 3×10 minutes in methanol, 3×10 minutes in 200 proof ethanol, and then dried overnight under vacuum. Disks were sterilized by dipping into 70% ethanol and drying in sterile vials in a sterile hood. Disks were stored at room temperature prior to use.

### Example 5: Conjugation of Exemplary Compounds onto a Surface Via Plasma Treatment

The compounds described in this disclosure can be attached to surfaces with a variety of methods. In this example, an acrylate derivative is attached to a polymer surface via plasma treatment. For example, compounds 302 and 303 may be conjugated to a polymer surface using this method. The polymeric material or device may be treated with plasma for set time period (e.g., 1 minute of each side (Harrick Plasma Cleaner)) and immediately dropped into a solution of the compound (e.g., a compound of Formula (I)) in 5% DMSO in toluene (0.2M overall). The reaction can be stirred or shaken (as appropriate) for 1 h. The materials will be filtered out of the solution and washed with methanol (3×), ethanol (3×) and dried under vacuum.

### Example 6: Preparation of Exemplary Cells for Encapsulation in Hydrogel Capsules

Engineered ARPE-19 cells for encapsulation as single cells.

ARPE-19 cells engineered to express a therapeutic agent, e.g., a blood clotting factor (e.g., a FVIII or FIX protein) may be cultured according to any method known in the art, such as according to the following protocol.

Engineered ARPE-19 cells in a 75 cm² culture flask were aspirated to remove culture medium, and the cell layer was briefly rinsed with 0.05% (w/v) trypsin/0.53 mM EDTA solution (“TrypsinEDTA”) to remove all traces of serum containing a trypsin inhibitor. Two to three mL of Trypsin/EDTA solution was added to the flask, and the cells were observed under an inverted microscope until the cell layer was dispersed, usually between 5-15 minutes. To avoid clumping, cells were handled with care and hitting or shaking the flask during the dispersion period was minimized. If the cells did not detach, the flasks were placed at 37° C. to facilitate dispersal. Once the cells dispersed, 6-8 mL complete growth medium was added and the cells were aspirated by gentle pipetting. The cell suspension was transferred to a centrifuge tube and spun down at approximately 125×g for 5-10 minutes to remove TrypsinEDTA. The supernatant was discarded, and the cells were re-suspended in fresh growth medium. Appropriate aliquots of cell suspension was added to new culture vessels, which were incubated at 37° C. The medium was renewed 2-3 times weekly.

ARPE-19 Cells for Encapsulation as Clusters

Spheroid clusters of exemplary cells (e.g., engineered ARPE-19 cells) are prepared using AggreWell™ spheroid plates (STEMCELL Technologies) and the protocol outlined herein. On Day 1, rinsing solution (4 mL) is added to each plate, and the plates is spun down for 5 minutes at 3,000 RPM in a large centrifuge. The rinsing solution is removed by pipet, and 4 mL of the complete growth medium is added. The engineered ARPE-19 cells are seeded into the plates at the desired cell density and pipetted immediately to prevent aggregation, with the general rule of thumb that 3.9 million cells per well will generate 150 μm diameter clusters. The plate is spun down for 3 minutes at 800 RPM, and the plate is placed into an incubator overnight at 37° C.

On Day 2, the plate is removed from incubation. Using wide bore pipet tips, the cells are gently pipetted to dislodge the spheroid clusters. The clusters are filtered through a 40 μm or 80 μm cell strainer to remove extraneous detached single cells and then spun down in a centrifuge for 2×1 minute. The clusters are resuspended gently using wide bore pipet tips and are gently stirred to distribute them throughout the medium or another material (e.g., alginate). Alternatively, ARPE-19 spheroids are prepared using the following protocol. On Day 1, AggreWell™ plates are removed from the packaging in a sterile tissue culture hood. 2 mL of Aggrewell™ Rinsing solution is added to each well. The plate is centrifuged at 2,000 g for 5 minutes to remove air bubbles, and the AggreWell™ Rinsing Solution is removed from the wells. Each well is rinsed with 2 mL of the complete growth medium, and 2 million engineered ARPE-19 cells in 3.9 mL of the complete growth medium is added to each well. The plate is centrifuged at 100 g for 3 minutes, then the cells are incubated the cells at 37° C. for 48 hours. On Day 3, the same protocol described above is used to dislodge the spheroid clusters. Alternatively, ARPE19 spheroids are prepared using a PBS MINI bioreactor (PBS Biotec, Inc., Camarillo Calif., USA) with the following protocol. Cell culture media and 220 million ARPE19 cells are added into a PBS 0.1 L or PBS 0.5 L vessel which is then inserted into the base unit which is placed in an incubator. The PBS MINI speed adjust dial is set at 40 rpm and the vessel is incubated at 37° C. for at least 48 hours prior to collection of spheroids as described above.

### Example 7: Preparation of Hydrogel Capsules Comprising Cells

Capsules encapsulating RPE cells as single cells. Immediately before encapsulation, single ARPE-19 cells engineered to express a therapeutic protein were centrifuged at 1,400 r.p.m. for 1 min and washed with calcium-free Krebs-Henseleit (KH) Buffer (4.7 mM KCl, 25 mM HEPES, 1.2 mM KH₂PO₄, 1.2 mM MgSO₄×7H₂O, 135 mM NaCl, pH≈7.4, ≈290 mOsm). After washing, the cells were centrifuged again and all of the supernatant was aspirated. In some experiments, the cell pellet was then resuspended in the 70:30 CM-LMW-Alg:U-HMW-Alg solution described in Example 2 (control capsules) at the desired density of suspended single cells per ml alginate solution.

Prior to fabrication of one-compartment and two-compartment hydrogel capsules, buffers and alginate solutions were sterilized by filtration through a 0.2-μm filter using aseptic processes.

To prepare devices configured as two-compartment hydrogel millicapsules of about 1.5 mm diameter, an electrostatic droplet generator was set up as follows: an ES series 0-100-kV, 20-watt high-voltage power generator (EQ series, Matsusada, N.C., USA) was connected to the top and bottom of a coaxial needle (inner lumen of 22G, outer lumen of 18G, Rame-Hart Instrument Co., Succasunna, N.J., USA). The inner lumen was attached to a first BD disposable 5-ml syringe with BD Luer-Lok™ tip (BD (Cat. No. 309646), Franklin Lakes, N.J., USA), which was connected to a syringe pump (Pump 11 Pico Plus, Harvard Apparatus, Holliston, Mass., USA) that was oriented vertically. The outer lumen was connected via a luer coupling to a second 5-ml Luer-lock syringe which was connected to a second syringe pump (Pump 11 Pico Plus) that was oriented horizontally. To encapsulate cells only in the first (inner) compartment, a first alginate solution comprising the cells (as single cell suspension) was placed in the first syringe and a second cell-free alginate solution comprising a compound of Formula (II) was placed in the second syringe. For control 2-compartment hydrogel capsules in the Examples below, the second (outer) compartment was formed using an alginate solution that did not comprise a compound of Formula (II). The two syringe pumps move the first and second alginate solutions from the syringes through both lumens of the coaxial needle and single droplets containing both alginate solutions are extruded from the needle into a glass dish containing a cross-linking solution. The settings of each Pico Plus syringe pump were 12.06 mm diameter and the flow rates of each pump were adjusted to achieve a flow rate ratio of 1:1 for the two alginate solutions. Thus, with the total flow rate set at 10 ml/h, the flow rate for each alginate solution was about 5 mL/h.

For fabrication of the two-compartment capsules, after extrusion of the desired volumes of alginate solutions, the alginate droplets were crosslinked for five minutes in a cross-linking solution which contained 25 mM HEPES, 20 mM BaCl₂, 0.2M mannitol, and poloxamer 188. Capsules that had fallen to the bottom of the crosslinking vessel were collected by pipetting into a conical tube. After the capsules settled in the tube, the crosslinking buffer was removed, and capsules were washed. Capsules without cells were washed four times with HEPES buffer (NaCl 15.428 g, KCl 0.70 g, MgCl₂.6H₂O 0.488 g, 50 ml of HEPES (1 M) buffer solution (Gibco, Life Technologies, California, USA) in 2 liters of deionized water) and stored at 4° C. until use. Capsules encapsulating cells were washed four times in HEPES buffer, two times in 0.9% saline, and two times in culture media and stored in an incubator at 37° C.

### Example 9. In Vivo Assay of Exemplary Compounds

The afibrotic properties of exemplary compounds of the present disclosure (Compounds 400 and 401) and previously described compounds (Compounds 402, 403, 404 and 405) were interrogated by implanting hydrogel capsules prepared as described in Example 8 into the intraperitoneal (IP) space of C57BL/6J mice according to the procedure below.

Preparation: Mice were prepared for surgery by being placed under anesthesia under a continuous flow of 1-4% isofluorane with oxygen at 0.5 L/min. Preoperatively, all mice received a 0.05-0.1 mg/kg of body weight dose of buprenorphine subcutaneously as a pre-surgical analgesic, along with 0.5 ml of 0.9% saline subcutaneously to prevent dehydration. A shaver with size #40 clipper blade was used to remove hair to reveal an area of about 2 cm×2 cm on ventral midline of the animal abdomen. The entire shaved area was aseptically prepared with a minimum of 3 cycles of scrubbing with povidine (in an outward centrifugal direction from the center of the incision site when possible), followed by rinsing with 70% alcohol. A final skin paint with povidine was also applied. The surgical site was draped with sterile disposable paper to exclude surrounding hair from touching the surgical site, after disinfection of table top surface with 70% ethanol. Personnel used proper PPE, gowning, surgical masks, and surgical gloves.

Surgical procedure: A sharp surgical blade or scissor was used to cut a 0.5-0.75 mm midline incision through the skin and the linea alba into the abdomen of the subject mice. The surgeon attempted to keep the incision as small as possible. Flat sterile forceps were used to transfer one silicone disk or a 0.5 mL aliquot of each capsule composition into the peritoneal cavity of each mouse (4 mice per composition). The abdominal muscle was closed by suturing with 5-0 Ethicon black silk or PDS-absorbable 5.0-6.0 monofilament absorbable thread, and the external skin layer was closed using wound clips. Blood and tissue debris were removed from the surgical instruments between procedures and the instruments were also re-sterilized between animal using a hot bead sterilizer. After the surgery, the animals were put back in the cage on a heat pad or under a heat lamp and monitored until they came out of anesthesia.

Intraoperative care: Animals were kept warm using Deltaphase isothermal pad. The animal's eyes were hydrated with sterile ophthalmic ointment during the period of surgery. Care was taken to avoid wetting the surgical site excessively to avoid hypothermia. Respiratory rate and character were monitored continuously. If vital signs are indicative of extreme pain and distress, the animal was euthanized in a carbon dioxide chamber followed by cervical dislocation.

Post-operative analysis: At 4 weeks post-implantation, the large majority of the capsules were retrieved from the mice and capsule cell numbers (one capsule in duplicate for each mouse) was measured using a CellTiter Glo® 3D Cell Viability Assay (Promega Corporation, Madison, Wis. USA). Briefly, one capsule per well was analyzed in duplicate and compared to a standard curve of plated cells. 100 μl of the CellTiter Glo® 3D reagent was added to the each well containing 100 μl of medium, the plate was placed onto a shaker at 400 rpm for 15 minutes and then luminescence was read on a plate reader. Also, a texture analyzer was used to measure the mechanical strength (initial fracture) of the capsules in aliquots of each composition at pre-implantation and upon retrieval after the 1-month implantation period.

In order to asses the overall afibrotic effect of each compound, the adhered tissue on each capsule was counted, averaged, and assigned a scoring value between 1.0 and 4.0, wherein 4.0 represents the afibrotic effect of the negative control (i.e., no afibrotic effect). The results of this assay are summarized in Table 3 below, in which “A” corresponds to a scoring value of 1.0 to 2.0; “B” corresponds to a scoring value of 2.0-3.0; and “C” corresponds to a scoring value of 3.0-4.0. In this assay, all six compounds exhibited an afibrotic effect compared to the negative control, with Compounds 400, 401 and 403, showing a greater afibrotic effect than Compounds 402, 404 and 405, and Compound 400 showing a marginally lower afibrotic effect than Compounds 401 and 403.

TABLE 3

| Compound | Assay |
|---|---|
| Number | Results |
| 400 | A/B |
| 401 | A |
| 402 | C |
| 403 | A |
| 404 | C |
| 405 | C |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (I-j): 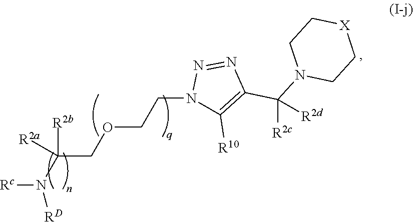
or a pharmaceutically acceptable salt thereof, wherein:
X is C(R′)(R″), N(R′), or S(O)ₓ;
each of R′ and R″ is independently hydrogen, alkyl, or halogen;
each of R^{2a}, R^{2b}, R^{2c}, and R^{2d} is independently hydrogen, alkyl, heteroalkyl, or halogen; or R^{2a} and R^{2b} or R^{2e} and R^{2d} are taken together to form an oxo group;
each of R^{C} and R^{D} is independently hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶;
each of R⁶ is independently alkyl, heteroalkyl, halogen, oxo, —OR^{A1}, —C(O)OR^{A1}, —C(O)R^{B1};
R¹⁰ is deuterium, alkyl, heteroalkyl, halo, cyano, azido, nitro, or amino, wherein each alkyl or heteroalkyl is optionally substituted by one or more R¹¹;
each R¹¹ is independently alkyl, alkenyl, alkynyl, heteroalkyl, halogen, cyano, oxo, hydroxyl, cycloalkyl, or heterocyclyl;
each R^{A1} and R^{B1} is independently hydrogen, alkyl, or heteroalkyl;
n is 1, 2, 3, 4, 5, or 6;
q is an integer from 0 to 25; and
x is 0, 1, or 2.

2. The compound of claim 1, wherein X is S(O)ₓ.

3. The compound of claim 2, wherein x is 2.

4. The compound of claim 1 or 2, wherein X is S(O)₂.

5. The compound of claim 1, wherein each of R^{2a}, R^{2b}, R^{2c}, and R^{2d} is independently hydrogen.

6. The compound of claim 1, wherein each of R^{C} and R^{D} is independently hydrogen, —C(O)(C₁-C₆-alkyl), or —C(O)(C₁-C₆-alkenyl).

7. The compound of claim 6, wherein each of R^{C} and R^{D} is hydrogen.

8. The compound of claim 1, wherein one of R^{C} and R^{D} is hydrogen and the other of R^{C} and R^{D} is —C(O)C(CH₃)(═CH₂).

9. The compound of claim 1, wherein R¹⁰ is deuterium, alkyl, heteroalkyl, halo, cyano, azido, nitro, or amino.

10. The compound of claim 1, wherein R¹⁰ is deuterium.

11. The compound of claim 1, wherein R¹⁰ is alkyl optionally substituted by one or more R¹¹ (—CH₃, —CH₂CH₃, —CF₃, —CH₂F, —CHF₂).

12. The compound of claim 1, wherein R¹⁰ is halo (e.g., Cl, Br, F).

13. The compound of claim 1, wherein n is 1.

14. The compound of claim 1, wherein q is 2, 3, 4, or 5 (e.g., 3).

15. The compound of claim 1, wherein:
X is S(O)₂;
each of R^{2a}, R^{2b}, R^{2c}, and R^{2d} is independently hydrogen;
each of R^{C} and R^{D} is independently hydrogen, —C(O)(C₁-C₆-alkyl), or —C(O)(C₁-C₆-alkenyl);
R¹⁰ is deuterium, alkyl (—CH₃, —CH₂CH₃, —CF₃, —CH₂F, —CHF₂), or halo (e.g., F, Cl, or Br);
n is 1; and
q is 3.

16. The compound of any one of claims 1-12, wherein the compound of Formula (I-j) is a compound listed in Table 1, or a pharmaceutically acceptable salt thereof.

17. The compound of claim 1, wherein the compound of Formula (I-j) is Compound 300 or Compound 302.

18. A compound of Formula (I-k): 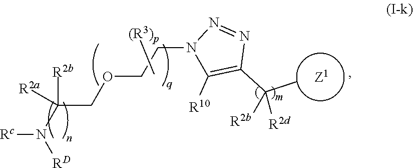
or a pharmaceutically acceptable salt thereof, wherein:
Ring Z¹ is heterocyclyl optionally substituted with 1-5 R⁵;
each of R^{C} and R^{D} is independently hydrogen, alkyl, —N(R^{c})C(O)R^{B}, —N(R^{c})C(O)(C₁-C₆-alkyl), or —N(R^{C})C(O)(C₁-C₆-alkenyl), wherein each of alkyl and alkenyl is optionally substituted with 1-6 R⁶;
each of R^{2a}, R^{2b}, R^{2c}, and R^{2d} is independently hydrogen or alkyl; or R^{2a} and R^{2b} or R^{2c} and R^{2d} are taken together to form an oxo group;
each of R⁵ and R⁶ is independently alkyl, heteroalkyl, halogen, oxo, —S(O)ₓR^{E1}, or —OS(O)ₓR^{E1};
R¹⁰ is hydrogen, deuterium, alkyl, heteroalkyl, halo, cyano, azido, nitro, or amino, wherein each alkyl or heteroalkyl is optionally substituted by one or more R¹¹;
each R¹¹ is independently alkyl, alkenyl, alkynyl, heteroalkyl, halogen, cyano, oxo, hydroxyl, cycloalkyl, or heterocyclyl;
each R^{A1}, R^{B1}, and R^{E1} is independently hydrogen, alkyl, or heteroalkyl; n is 1, 2, 3, 4, 5, or 6;
q is an integer from 0 to 25;
x is 0, 1, or 2; and
z is 0, 1, 2, 3, 4, 5, or 6.

19. The compound of claim 18, wherein Ring Z¹ is heterocyclyl.

20. The compound of claim 19, wherein Ring Z¹ is heterocyclyl substituted with 1 R⁵ (e.g., —S(O)₂(CH₃)).

21. The compound of claim 18, wherein each of R^{2a}, R^{2b}, R^{2c}, and R^{2d} is independently hydrogen.

22. The compound of claim 18, wherein each of R^{C} and R^{D} is independently hydrogen, —C(O)(C₁-C₆-alkyl), or —C(O)(C₁-C₆-alkenyl).

23. The compound of claim 18, wherein each of R^{C} and R^{D} is hydrogen.

24. The compound of claim 18, wherein one of R^{C} and R^{D} is hydrogen and the other of R^{C} and R^{D} is —C(O)C(CH₃)(═CH₂).

25. The compound of claim 18, wherein R⁵ is —S(O)ₓR^{E1}.

26. The compound of claim 25, wherein R^{E1} is alkyl (e.g., —CH₃).

27. The compound of claim 18, wherein R⁵ is —S(O)₂(CH₃).

28. The compound of claim 18, wherein R¹⁰ is hydrogen, deuterium, alkyl, heteroalkyl, halo, cyano, azido, nitro, or amino.

29. The compound of claim 18, wherein R¹⁰ is hydrogen or deuterium.

30. The compound of claim 18, wherein R¹⁰ is alkyl optionally substituted by one or more R¹¹ (—CH₃, —CH₂CH₃, —CF₃, —CH₂F, —CHF₂).

31. The compound of claim 18, wherein R¹⁰ is halo (e.g., Cl, Br, F).

32. The compound of claim 18, wherein z is 1.

33. The compound of claim 18, wherein n is 1.

34. The compound of claim 18, wherein q is 2, 3, 4, or 5 (e.g., 3).

35. The compound of claim 18, wherein:
Ring Z¹ is heterocyclyl substituted with 1 R⁵;
R⁵ is —S(O)₂(CH₃);
each of R^{2a}, R^{2b}, R^{2c}, and R^{2d} is independently hydrogen;
R¹⁰ is hydrogen, deuterium, alkyl, heteroalkyl, halo, cyano, azido, nitro, or amino, wherein each alkyl or heteroalkyl is optionally substituted by one or more R¹¹;
each R¹¹ is independently alkyl, alkenyl, alkynyl, heteroalkyl, halogen, cyano, oxo, hydroxyl, cycloalkyl, or heterocyclyl;
each of R^{C} and R^{D} is independently hydrogen, —C(O)(C₁-C₆-alkyl), or —C(O)(C₁-C₆-alkenyl);
z is 1;
n is 1; and
q is 3.

36. The compound of claim 18, wherein the compound of Formula (I-k) is a compound in Table 1, or a pharmaceutically acceptable salt thereof.

37. The compound of claim 18, wherein the compound of Formula (I-k) is Compound 301 or Compound 303, or a pharmaceutically acceptable salt thereof.

38. A polymer modified with a compound of Formula (II-j): 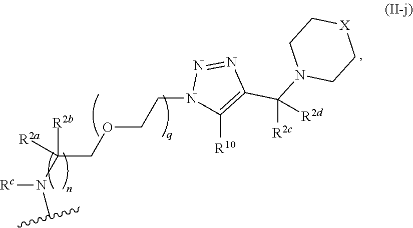
or a pharmaceutically acceptable salt thereof, wherein:
X is C(R′)(R″), N(R′), or S(O)ₓ;
each of R′ and R″ is independently hydrogen, alkyl, or halogen;
each of R^{2a}, R^{2b}, R^{2c}, and R^{2d} is independently hydrogen, alkyl, heteroalkyl, or halogen;
or R^{2a} and R^{2b} or R^{2e} and R^{2d} are taken together to form an oxo group;
R^{C} is independently hydrogen, alkyl, —N(R^{c})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶;
each of R⁶ is independently alkyl, heteroalkyl, halogen, oxo, —OR^{A1}, —C(O)OR^{A1}, —C(O)R^{B1};
R¹⁰ is deuterium, alkyl, heteroalkyl, halo, cyano, azido, nitro, or amino, wherein each alkyl or heteroalkyl is optionally substituted by one or more R¹¹;
each R¹¹ is independently alkyl, alkenyl, alkynyl, heteroalkyl, halogen, cyano, oxo, hydroxyl, cycloalkyl, or heterocyclyl;
each R^{A1} and R^{B1} is independently hydrogen, alkyl, or heteroalkyl;
n is 1, 2, 3, 4, 5, or 6;
q is an integer from 0 to 25; and
x is 0, 1, or 2.

39. The polymer of claim 38, wherein X is S(O)ₓ.

40. The polymer of claim 39, wherein x is 2.

41. The polymer of claim 38, wherein X is S(O)₂.

42. The polymer of claim 38, wherein each of R^{2a}, R^{2b}, R^{2c}, and R^{2d} is independently hydrogen or alkyl (C₁-C₆ alkyl).

43. The polymer of claim 38, wherein each of R^{2a}, R^{2b}, R^{2c}, and R^{2d} is independently hydrogen.

44. The polymer of claim 38, wherein n is 1 or 2.

45. The polymer of claim 38, wherein n is 1.

46. The polymer of claim 38, wherein R^{C} is hydrogen.

47. The polymer of claim 38, wherein q is 1, 2, 3, or 4.

48. The polymer of claim 38, wherein q is 3.

49. The polymer of claim 38, wherein R⁵ is-S(O)ₓR^{E1}.

50. The polymer of claim 38, wherein R⁵ is —S(O)₂R^{E1}, and R^{E1} is alkyl (e.g., —CH₃).

51. The polymer of claim 38, wherein R⁵ is —S(O)₂CH₃, and z is 1.

52. The polymer of claim 38, wherein R¹⁰ is deuterium, alkyl, heteroalkyl, halo, cyano, azido, nitro, or amino.

53. The polymer of claim 38, wherein R¹⁰ is deuterium.

54. The polymer of claim 38, wherein R¹⁰ is alkyl optionally substituted by one or more R¹¹ (—CH₃, —CH₂CH₃, —CF₃, —CH₂F, —CHF₂).

55. The polymer of claim 38, wherein R¹⁰ is halo (e.g., Cl, Br, F).

56. The polymer of claim 38, wherein:
R⁵ is —S(O)₂(CH₃);
each of R^{2a}, R^{2b}, R^{2c}, and R^{2d} is independently hydrogen;
R¹⁰ is deuterium, alkyl (—CH₃, —CH₂CH₃, —CF₃, —CH₂F, —CHF₂), or halo (e.g., F, Cl, or Br);
R^{C} is hydrogen;
z is 1;
n is 1; and
q is 3.

57. The polymer of claim 38, wherein the compound is a compound in Table 2 or a pharmaceutically acceptable salt thereof.

58. The polymer of claim 38, wherein the compound is Compound 401 or a pharmaceutically acceptable salt thereof.

59. The polymer of any one of claims 38-58, wherein the polymer is a hydrogel-forming polymer.

60. The polymer of any one of claims 38-58, wherein the polymer is a polysaccharide.

61. The polymer of any one of claims 38-58, wherein the polymer selected from alginate, hyaluronate, or chitosan.

62. The polymer of any one of claims 38-58, wherein the polymer is alginate.

63. The polymer of any one of claims 38-58, wherein the polymer is a high guluronic acid (G) alginate or a high mannuronic acid (M) alginate.

64. The polymer of any one of claims 38-58, wherein the polymer comprises an increase in % N (as compared with unmodified polymer) of 0.1 to 10% N by weight, where % N is determined by elemental analysis and corresponds to the amount of compound of Formula (II) in the modified polymer.

65. The polymer of any one of claims 38-58, wherein the polymer comprises an increase in % N (as compared with unmodified polymer) of 1 to 10% N by weight, where % N is determined by elemental analysis and corresponds to the amount of compound of Formula (II) in the modified polymer.

66. The polymer of any one of claims 38-58, wherein the polymer comprises an increase in % N (as compared with unmodified polymer) of 2 to 8% N by weight, where % N is determined by elemental analysis and corresponds to the amount of compound of Formula (II) in the modified polymer.

67. The polymer of any one of claims 38-58, comprising about 5% to 50% (w/w) of a compound of Formula (II-j) conjugated to the polymer.

68. The polymer of any one of claims 38-58, comprising about 10% to 40% (w/w) of a compound of Formula (II-j) conjugated to the polymer.

69. The polymer of any one of claims 38-58, comprising about 20% to 30% (w/w) of a compound of Formula (II-j) conjugated to the polymer.

70. An alginate modified with Compound 401 or a pharmaceutically acceptable salt thereof.

71. An alginate modified with Compound 402 or a pharmaceutically acceptable salt thereof.

72. The alginate of claim 70 or 71, which is a high guluronic acid (G) alginate.

73. An implantable element comprising a compound of any one of claims 1-37 or a polymer of any one of claims 38-58.

74. The implantable element of claim 73, wherein the compound is disposed on a surface, e.g., an inner or outer surface, of a device.

75. The implantable element of claim 73, wherein the compound is selected from a compound in Table 2 or a pharmaceutically acceptable salt thereof.

76. The implantable element of claim 73, wherein the implantable element (e.g., device or material) comprises a cell, e.g., a recombinant cell, which provides a substance, e.g., a therapeutic agent.

77. The implantable element of claim 76, wherein the cell comprises an exogenous nucleic acid (e.g., RNA (e.g., an mRNA) or DNA) that encodes a polypeptide.

78. The implantable element of claim 77, wherein the polypeptide comprises a clotting factor, a hormone or an an enzyme.

79. The implantable element of claim 77, wherein the polypeptide comprises a Factor VIII protein or a Factor IX protein.

80. The implantable element of claim 73, wherein the implantable element is formulated for implantation into a subject (e.g., into the intraperitoneal (IP) space, the peritoneal cavity, the omentum, the lesser sac, the subcutaneous fat).

81. The implantable element of claim 80, wherein the implantable element is formulated for implantation into the IP space of a subject.

82. The implantable element of claim 67, wherein the compound is Compound 401, Compound 402 or a pharmaceutically acceptable salt of Compound 401 or Compound 402.

83. A pharmaceutical composition comprising a compound of any one of claims 1-37, a polymer of any one of claims 38-58, or an implantable element of any one of claims 73-82 and a pharmaceutically acceptable excipient.

84. A pharmaceutical composition comprising the alginate of any one of claims 70-72 and a pharmaceutically acceptable excipient.

85. A method of providing a substance to a subject, comprising administering to the subject an implantable element of any one of claims 73-82.

86. A method of treating a disease, disorder, or condition in a subject with a therapeutic agent, comprising administering to the subject an implantable element of any one of claims 73-82.

87. The method of claim 86, wherein the administering comprises implanting an effective amount of the implantable element into the IP space of the subject.
